SEQUENCE LISTING (146 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 125 aa, Artificial Sequence
  VHH72 amino acid sequence
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 3 - protein, 125 aa, Artificial Sequence
  VHH72-S56A amino acid sequence
QVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWDYDYDYWGQGTQVTVSS

SEQ ID NO: 4 - protein, 125 aa, Artificial Sequence
  VHH72_h1(S56A) humanized variant 1of VHH72-S56A amino acid sequence
EVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 5 - protein, 125 aa, Artificial Sequence
  VHH72_h1(E1D)(S56A) humanized variant 1(E1D) of VHH72-S56A amino acid sequence
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 6 - protein, 127 aa, Artificial Sequence
  VHH3-83 amino acid sequence
QVQLQESGGGLVQPGDSLRLSCVLSGGVFTSYAMGWFRQAPGKEREFLAAITFNSDATYYADSVKGRFTISRDNAKNTAYLQMNSLKPNDTAVYSCAAGGNHYNPQYYHDYDKYDHWGQGTQVTVSS

SEQ ID NO: 7 - protein, 127 aa, Artificial Sequence
  VHH3.83 variant VHH3-83-hc amino acid sequence
DVQLQESGGGLVQPGDSLRLSCVLSGGVFTSYAMGWFRQAPGKEREFLAAITFNSDATYYADSVKGRFTISRDNAKNTAYLQMNSLRPNDTAVYSCAAGGNHYNPQYYHDYDKYDHWGQGTLVTVSS

SEQ ID NO: 8 - protein, 127 aa, Artificial Sequence
  VHH3.83 variant VHH3-83-hc-N85E amino acid sequence
DVQLQESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 9 - protein, 125 aa, Artificial Sequence
  VHH2.50 amino acid sequence
QVQLQESGGGLVQAGGSLRLSCAASGRTFSSIAMGWFRQAPGKEREFVATISWSGGSTYYADSVKGRFTISRDNAKNTVYLQMNSLKPEDTAVYYCAAAGLGTVVSEWDYDYDYWGQGTQVTVSS

SEQ ID NO: 10 - protein, 125 aa, Artificial Sequence
  VHH3.17 amino acid sequence
QVQLQESGGGLVEAGGSLTLSCAASGRAFGDGAVGWFRQGPGRPREFVATVSWNGGGTYFAESVRGRFTISRDNAKNTVYLQMNNLKPEDTAVYYCALAGEGTVVSEWDYDYEYWGQGTQVTVSS

SEQ ID NO: 11 - protein, 125 aa, Artificial Sequence
  VHH3.77 amino acid sequence
QVQLQESGGGLVQAGGSLRLSCVSSGRAFGNGAVGWFRQGPGRPREFVATVSWNGGGTYFAESVRGRFTISRDNAKNTVYLQMNNLKPEDTAVYYCALAGEGTVVSEWDYDYEYWGQGTQVTVSS

SEQ ID NO: 12 - protein, 125 aa, Artificial Sequence
  VHH3.115 amino acid sequence
QVQLQESGGGLVQTGGSLRLSCAASGLTFSDIAMGWFRQAPGREREFVATISWSGGGTYYAEPVRGRFAISRDNAKNTVYLQMNSLKPEDTAVYYCAAAGAGTVVSEWDYDYDYWGQGTQVTVSS

SEQ ID NO: 13 - protein, 125 aa, Artificial Sequence
  VHH3.144 amino acid sequence
QVQLQESGGGLVQAGGSLRLSCVASGRAFGNGAVGWFRQAPGREREFVATVSWNGGGTYYAESVRGRFTISRDNAKSTVYLQMNSLKPEDTAVYYCASAGEGTVVSEWDYDYDYWGQGTQVTVSS

SEQ ID NO: 14 - protein, 125 aa, Artificial Sequence; identical to SEQ ID NO: 13
  VHH3BE4 amino acid sequence

SEQ ID NO: 15 - protein, 121 aa, Artificial Sequence
  VHH3.36 amino acid sequence
QVQLQESGGGLVQAGDSLRLSCVVSGGTFSSYAMGWFRQAPGKERELVAAINWGGISVYYADSVKGRFTISRDNAENTVYLQMNSLKPEDTAVYYCAADPKGWSEWDMEYWGKGTQVTVSS

SEQ ID NO: 16 - protein, 121 aa, Artificial Sequence
  VHH3.47 amino acid sequence
QVQLQESGGGLVQAGDSLRLSCAASGHNFSTYAMAWFRQAPGTERELVAAISENDVMRYYADSVKGRFTISRDNAKNMAYLQMNSLKVEDTAVYLCAADPKGWSEWDMDYWGKGTQVTVSS

SEQ ID NO: 17 - protein, 121 aa, Artificial Sequence
  VHH3.55 amino acid sequence
QVQLQESGGGLVQAGGSLRLSCEVSGRTNDNYGVGWFRQVPGAERELVAAIRWSSISRYYKDSVKGRFTISRDNAKNTVYLQMDMLKPEDTAAYYCAADPAGWSEFGMEYWGKGTQVTVSS

SEQ ID NO: 18 - protein, 121 aa, Artificial Sequence
  VHH3.35 amino acid sequence
QVQLQESGGGLVQAGGSLTLSCEVSGRTNDNYGVGWFRQVPGAERELVAAIRWSSISRYYKDSVKGRFTISRDNAKNTVYLQMDMLKAEDTAVYYCAADPAGWSEFGMEYWGKGTQVTVSS

SEQ ID NO: 19 - protein, 121 aa, Artificial Sequence
  VHH3.29 amino acid sequence
QVQLQESGGGLVQAGGSLRLSCVASGRTFSSGGMGWFRQAPGQEREFVAGIGWAGLSSYYLDSVKGRFTISLDNAKNTVYLQMNMLKPEDTGVYYCAADDHGWSAAGMDYLGQGTQVTVSS

SEQ ID NO: 20 - protein, 122 aa, Artificial Sequence
  VHH3.38 amino acid sequence
QVQLQESGGGLVQAGGSLRLSCAASGGTFNNYAMAWFRQAPGQERELVAAMFWSGLPKYYADSVKGRFTISTDDAKNTVYLQMNGLKPEDTAVYYCAADSRGWSDVGGMDYWGKGTQVTVSS

SEQ ID NO: 21 - protein, 121 aa, Artificial Sequence
  VHH3.149 amino acid sequence
QVQLQESGGGLVQAGGSLKLSCAASGRTFSSYALGWFRQAPGTEREFVSAINWFGAPTYYADSVKGRFTISRDNAKNTVYLQMDNLKPEDTAVYYCAADSKGWDPQDMDYWGKGTQVTVSS

SEQ ID NO: 22 - protein, 123 aa, Artificial Sequence
  VHH3.117 amino acid sequence
QVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSS

SEQ ID NO: 23 - protein, 123 aa, Artificial Sequence
  VHH3_117-hc amino acid sequence
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSS

SEQ ID NO: 24 - protein, 123 aa, Artificial Sequence
  VHH3.92 amino acid sequence
QVQLQESGGGLVQPGGSLRLSCAASGKAVSISDMGWYRQPPGKQRELVATITKTGNTNYADSAQGRFTISRDNAKSAVYLEMASLKPEDTAVYYCNAWLPYGMGPDYYGMELWGKGTQVTVSS

SEQ ID NO: 25 - protein, 123 aa, Artificial Sequence
  VHH3.94 amino acid sequence
QVQLQESGGGLVQPGGSLRLSCAASGKAVSISDMGWYRQPPGKQRELVATITKSGSTNYANSAQGRFTISRDNAKSAVYLEMNSLKPEDTAVYYCNAWLPYGMGPDYYGMELWGEGTQVTVSS

SEQ ID NO: 26 - protein, 123 aa, Artificial Sequence
  VHH3.42 amino acid sequence
QVQLQESGGGLVQPGGSLRLSCAASGSAVSINDMGWYRQPPGKQRELVATITKTGSTNYADSVKGRFTISRDNAKNAVYLEMNSLKPEDTATYYCNAWLPYGMGPDYYGMELWGKGTQVTVSS

SEQ ID NO: 27 - protein, 123 aa, Artificial Sequence
  VHH3.180 amino acid sequence
QVQLQESGGGSVQAGRSLTLNCAASGKAVSISDMGWYRQPPGKQRELVATITKTGSTNYADSAQGRFTISRDNAKSAVYLEMNSLKPEDTAVYYCNAWLLYGMGPDYYGMELWGEGTQVTVSS

SEQ ID NO: 28 - protein, 5 aa, Artificial Sequence
  binder
EYAMG

SEQ ID NO: 29 - protein, 5 aa, Artificial Sequence
  binder
SYAMG

SEQ ID NO: 30 - protein, 5 aa, Artificial Sequence
  binder
SIAMG

SEQ ID NO: 31 - protein, 5 aa, Artificial Sequence
  binder
DGAVG

SEQ ID NO: 32 - protein, 5 aa, Artificial Sequence
  binder
NGAVG

SEQ ID NO: 33 - protein, 5 aa, Artificial Sequence
  binder
DIAMG

SEQ ID NO: 34 - protein, 5 aa, Artificial Sequence
  binder
NYGVG

SEQ ID NO: 35 - protein, 5 aa, Artificial Sequence
  binder
SGGMG

SEQ ID NO: 36 - protein, 5 aa, Artificial Sequence
  binder
NYAMA

SEQ ID NO: 37 - protein, 5 aa, Artificial Sequence
  binder
SYALG

SEQ ID NO: 38 - protein, 17 aa, Artificial Sequence
  binder
TISWSGGSTYYTDSVKG

SEQ ID NO: 39 - protein, 17 aa, Artificial Sequence
  binder
TISWSGGATYYTDSVKG

SEQ ID NO: 40 - protein, 17 aa, Artificial Sequence
  binder
AITFNSDATYYADSVKG

SEQ ID NO: 41 - protein, 17 aa, Artificial Sequence
  binder
TISWSGGSTYYADSVKG

SEQ ID NO: 42 - protein, 17 aa, Artificial Sequence
  binder
TVSWNGGGTYFAESVRG

SEQ ID NO: 43 - protein, 17 aa, Artificial Sequence
  binder
TISWSGGGTYYAEPVRG

SEQ ID NO: 44 - protein, 17 aa, Artificial Sequence
  binder
TVSWNGGGTYYAESVRG

SEQ ID NO: 45 - protein, 17 aa, Artificial Sequence
  binder
AINWGGISVYYADSVKG

SEQ ID NO: 46 - protein, 17 aa, Artificial Sequence
  binder
AISENDVMRYYADSVKG

SEQ ID NO: 47 - protein, 17 aa, Artificial Sequence
  binder
AIRWSSISRYYKDSVKG

SEQ ID NO: 48 - protein, 17 aa, Artificial Sequence
  binder
GIGWAGLSSYYLDSVKG

SEQ ID NO: 49 - protein, 17 aa, Artificial Sequence
  binder
AMFWSGLPKYYADSVKG

SEQ ID NO: 50 - protein, 17 aa, Artificial Sequence
  binder
AINWFGAPTYYADSVKG

SEQ ID NO: 51 - protein, 16 aa, Artificial Sequence
  binder
AGLGTVVSEWDYDYDY

SEQ ID NO: 52 - protein, 18 aa, Artificial Sequence
  binder
GGNHYNPQYYHDYDKYDH

SEQ ID NO: 53 - protein, 16 aa, Artificial Sequence
  binder
AGEGTVVSEWDYDYEY

SEQ ID NO: 54 - protein, 16 aa, Artificial Sequence
  binder
AGAGTVVSEWDYDYDY

SEQ ID NO: 55 - protein, 16 aa, Artificial Sequence
  binder
AGEGTVVSEWDYDYDY

SEQ ID NO: 56 - protein, 12 aa, Artificial Sequence
  binder
DPKGWSEWDMEY

SEQ ID NO: 57 - protein, 12 aa, Artificial Sequence
  binder
DPKGWSEWDMDY

SEQ ID NO: 58 - protein, 12 aa, Artificial Sequence
  binder
DPAGWSEFGMEY

SEQ ID NO: 59 - protein, 12 aa, Artificial Sequence
  binder
DDHGWSAAGMDY

SEQ ID NO: 60 - protein, 13 aa, Artificial Sequence
  binder
DSRGWSDVGGMDY

SEQ ID NO: 61 - protein, 12 aa, Artificial Sequence
  binder
DSKGWDPQDMDY

SEQ ID NO: 62 - protein, 6 aa, Artificial Sequence
  binder
ISDMGW

SEQ ID NO: 63 - protein, 6 aa, Artificial Sequence
  binder
INDMGW

SEQ ID NO: 64 - protein, 16 aa, Artificial Sequence
  binder
TITKTGSTNYADSAQG

SEQ ID NO: 65 - protein, 16 aa, Artificial Sequence
  binder
TITKTGNTNYADSAQG

SEQ ID NO: 66 - protein, 16 aa, Artificial Sequence
  binder
TITKSGSTNYANSAQG

SEQ ID NO: 67 - protein, 16 aa, Artificial Sequence
  binder
TITKTGSTNYADSVKG

SEQ ID NO: 68 - protein, 14 aa, Artificial Sequence
  binder
WLPYGMGPDYYGME

SEQ ID NO: 69 - protein, 14 aa, Artificial Sequence
  binder
WLLYGMGPDYYGME

SEQ ID NO: 70 - protein, 6 aa, Artificial Sequence
  amino acid consensus sequence of CDR1 for the VHH3.117 family
  MISC_FEATURE (2)..(2): X (Xaa) at position 2 is S or N
IXDMGW

SEQ ID NO: 71 - protein, 16 aa, Artificial Sequence
  amino acid consensus sequence of CDR2 for the VHH3.117 family
  MISC_FEATURE (5)..(5): X (Xaa) at position 5 is T or S
  MISC_FEATURE (7)..(7): X (Xaa) at position 7 is S or N
  MISC_FEATURE (12)..(12): X (Xaa) at position 12 is D or N
  MISC_FEATURE (14)..(14): X (Xaa) at position 14 is A or V
  MISC_FEATURE (15)..(15): X (Xaa) at position 15 is Q or K
TITKXGXTNYAXSXXG

SEQ ID NO: 72 - protein, 14 aa, Artificial Sequence
  amino acid consensus sequence of CDR3 for the VHH3.117 family
  MISC_FEATURE (3)..(3): X (Xaa) at position 3 is P or L
WLXYGMGPDYYGME

SEQ ID NO: 73 - protein, 280 aa, Artificial Sequence
  homobivalent VHH amino acid sequence encoded by construct pX-B001_GS-VHH72-h1-E1D-S56A_(G4S)6_VHH72-h1-E1D-S56A_His8
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSGGGGSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 74 - protein, 255 aa, Artificial Sequence
  homobivalent VHH amino acid sequence encoded by construct pX-B002_GS-VHH72-h1-E1D-S56A_(G4S)_VHH72-h1-E1D-S56A_His8
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 75 - protein, 270 aa, Artificial Sequence
  homobivalent VHH amino acid sequence encoded by construct pX-B003_GS-VHH72-h1-E1D-S56A_(G4S)4_VHH72-h1-E1D-S56A_His8
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 76 - protein, 278 aa, Artificial Sequence
  Bispecific VHH amino acid sequence encoded by construct pX-B004_GS-VHH3-117-hc_(G4S)6_VHH72-h1-E1D-S56A _His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 77 - protein, 253 aa, Artificial Sequence
  Bispecific VHH amino acid sequence encoded by construct pX-B005_GS-VHH3-117-hc_(G4S)_VHH72-h1-E1D-S56A _His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 78 - protein, 268 aa, Artificial Sequence
  Bispecific VHH amino acid sequence encoded by construct pX-B006_GS-VHH3-117-hc_(G4S)4_VHH72-h1-E1D-S56A _His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSS

SEQ ID NO: 79 - protein, 280 aa, Artificial Sequence
  Bispecific VHH amino acid sequence encoded by construct pX-B007_GS-VHH3-117-hc_(G4S)6_VHH3-83-hc_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSGGGGSGGGGSDVQLQESGGGLVQPGDSLRLSCVLSGGVFTSYAMGWFRQAPGKEREFLAAITFNSDATYYADSVKGRFTISRDNAKNTAYLQMNSLRPNDTAVYSCAAGGNHYNPQYYHDYDKYDHWGQGTLVTVSS

SEQ ID NO: 80 - protein, 280 aa, Artificial Sequence
  Bispecific VHH amino acid sequence encoded by construct pX-B008_GS-VHH3-117-hc_(G4S)6_VHH3-83-hc-N85E_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSGGGGSGGGGSDVQLQESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 81 - protein, 255 aa, Artificial Sequence
  Bispecific VHH amino acid sequence encoded by construct pX-B009_GS-VHH3-117-hc_(G4S)_VHH3-83-hc_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSDVQLQESGGGLVQPGDSLRLSCVLSGGVFTSYAMGWFRQAPGKEREFLAAITFNSDATYYADSVKGRFTISRDNAKNTAYLQMNSLRPNDTAVYSCAAGGNHYNPQYYHDYDKYDHWGQGTLVTVSS

SEQ ID NO: 82 - protein, 270 aa, Artificial Sequence
  Bispecific VHH amino acid sequence encoded by construct pX-B010_GS-VHH3-117-hc_(G4S)4_VHH3-83-hc_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSDVQLQESGGGLVQPGDSLRLSCVLSGGVFTSYAMGWFRQAPGKEREFLAAITFNSDATYYADSVKGRFTISRDNAKNTAYLQMNSLRPNDTAVYSCAAGGNHYNPQYYHDYDKYDHWGQGTLVTVSS

SEQ ID NO: 83 - protein, 255 aa, Artificial Sequence
  Bispecific VHH amino acid sequence encoded by construct pX-B011_GS-VHH3-117-hc_(G4S)_VHH3-83-hc-N85E_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSDVQLQESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 84 - protein, 270 aa, Artificial Sequence
  Bispecific VHH amino acid sequence encoded by construct pX-B012_GS-VHH3-117-hc_(G4S)4_VHH3-83-hc-N85E_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSDVQLQESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 85 - protein, 122 aa, Artificial Sequence
  VHH3.89
QVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSS

SEQ ID NO: 86 - protein, 120 aa, Artificial Sequence
  VHH3_183
QVQLQESGGGLVQPGGSLRLSCAASGLDYYAIGWFRQAPGKEREGLSRIESSDGSTYYADSVKGRFTISRDNTKNTVYLQMNSLKPEDTAVYYCATDPIIQGSSWYWTSWGQGTQVTVSS

SEQ ID NO: 87 - protein, 122 aa, Artificial Sequence
  VHH3C_80
QVQLQESGGGSVQPGESLRLSCVGSGHTLDDYDVGWFRQAPGKEREVLSRIDSSDGSTYYADSVKGRFTISRDNTKNIVYLQMNMLKPEDTAAYYCATDPIIRGHNWYWTGWSQSTHITVSS

SEQ ID NO: 88 - protein, 361 aa, Artificial Sequence
  Bivalent-Fc construct VHH72_h1_E1D_R27L_E31D_Y32I_S56G_L97A-10xGS-hIgG1_EPKSCdel_LALA_K 447del
DVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 364 aa, Artificial Sequence
  Bivalent-Fc construct VHH72-h1-E1D-R27L-E31D-Y32I-S56G-L97A_(GGGGS)x2_hIgGhingeEPKSCdel _hIgGFc_N297A_Gsdel
DVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSSGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 125 aa, Artificial Sequence
  VHH72-h1-E1D-R27L-E31D-Y32I-S56G-L97A
DVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSS

SEQ ID NO: 91 - protein, 278 aa, Artificial Sequence
  Bispecific VHH amino acid sequence of construct pX-B014_GS-VHH3_117-hc_(G4S)6_VHH72-h1-E1D-R27L-E31D-Y32I-S56G-L9 7A_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSGGGGSGGGGSDVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSS

SEQ ID NO: 92 - protein, 253 aa, Artificial Sequence
  Bispecific VHH amino acid sequence of construct pX-B017_GS-VHH3_117-hc_(G4S)_VHH72-h1-E1D-R27L-E31D-Y32I-S56G-L97 A_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSDVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSS

SEQ ID NO: 93 - protein, 268 aa, Artificial Sequence
  Bispecific VHH amino acid sequence of construct pX-B018_GS-VHH3_117-hc_(G4S)4_VHH72-h1-E1D-R27L-E31D-Y32I-S56G-L9 7A_His8
DVQLVESGGGLVQPGGSLRLSCAASGKAVSISDMGWYRQPPGKQRELVATITKTGSTNYADSAQGRFTISRDNTKSAVYLEMKSLRPEDTAVYYCNAWLPYGMGPDYYGMELWGKGTLVTVSSGGGGSGGGGSGGGGSGGGGSDVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSS

SEQ ID NO: 94 - protein, 364 aa, Artificial Sequence
  Bivalent VHH-Fc amino acid sequence VHH3.115-h1-E1D(GGGGS)x2_hIgGhingeEPKSCdel_hIgGFc_N297A_Gsdel
DVQLVESGGGLVQPGGSLRLSCAASGLTFSDIAMGWFRQAPGREREFVATISWSGGGTYYAEPVRGRFAISRDNAKNTVYLQMNSLRPEDTAVYYCAAAGAGTVVSEWDYDYDYWGQGTLVTVSSGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 127 aa, Artificial Sequence
  VHH4.1XAS51
QVQLQESGGGLVQAGGSLRLSCTASTPTIKNYAMGWFRQAPGKEREFLAAITFNSDATYYADSVKGRFTISRDNAKNTAYLQMNSLKPDDTAVYSCAAGGNHYNPQYYHDYDKYDYWGQGTQVTVSS

SEQ ID NO: 96 - protein, 127 aa, Artificial Sequence
  VHH4.2XAS58
QVQLQESGGGLVQSGGSLRLSCAGSGFPLEAFSIGWFRQAPGKEREFLAAITFNSDATYYADSVKGRFTISRDNAKNTAYLQMNSLKPDDTAVYSCAAGGNHYNPQYYHDYDKYDHWGQGTQVTVFS

SEQ ID NO: 97 - protein, 127 aa, Artificial Sequence
  VHH4.2XAS31
QVQLQESGGGFVQAGGSLRLSCATSGHRFSDYAVAWFRQTPGKEREFLAAITFNSDATYYADSVKGRFTISRDNAKNTAYLQMNSLKPDDTAVYSCAAGGNHYNPQYYHDYDKYDHWGQGTQVTVSS

SEQ ID NO: 98 - protein, 127 aa, Artificial Sequence
  VHH4.2XAS43
QVQLQESGGGLVQAGDSLTLSCAASGRTGSNYAMGWFRQAPGKEREFLAAITFNSDATYYADSVKGRFTISRDNAKNTAYLQMNSLKPDDTAVYSCAAGGNHYNPQYYHDYDKYDHWGQGTQVTVSS

SEQ ID NO: 99 - protein, 151 aa, Severe acute respiratory syndrome coronavirus 2
GVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 100 - protein, 186 aa, Severe acute respiratory syndrome coronavirus 2
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 101 - protein, 201 aa, SARS-CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 102 - protein, 359 aa, Artificial Sequence
  VHH117_Q1D-10xGS-hIgG1_EPKSCdel_LALA_K447del
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 363 aa, Artificial Sequence
  VHH83_Q1D-N85E_10xGS-hIgG1_EPKSCdel_LALA_K447del
DVQLQESGGGLVQPGDSLRLSCVLSGGVFTSYAMGWFRQAPGKEREFLAAITFNSDATYYADSVKGRFTISRDNAKNTAYLQMNSLKPEDTAVYSCAAGGNHYNPQYYHDYDKYDHWGQGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 358 aa, Artificial Sequence
  VHH89_Q1D-10xGS-hIgG1_EPKSCdel_LALA_K447del
DVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 361 aa, Artificial Sequence
  VHH72_h1_E1D_S56A-10xGS-hIgG1_EPKSCdel_LALA_K447del
DVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 367 aa, Artificial Sequence
  VHH23-GS(G4S)2-hIgG1hinge-hIgG1
QVQLQESGGGLVQTGGSLRLSCAFSGFTSDDYVIGWFRQAPGKGRQGVSCIRLSGGGTIYADSAKGRFTVSADNAKKTVYLQMTRLKPEDTAVYYCGAERYNVEGCGYDVAYWGKGTQVTVSSGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 359 aa, Artificial Sequence
  VHH3.117-holeFc
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 108 - protein, 363 aa, Artificial Sequence
  VHH83_Q1D_N85E-knobFc
DVQLQESGGGLVQPGDSLRLSCVLSGGVFTSYAMGWFRQAPGKEREFLAAITFNSDATYYADSVKGRFTISRDNAKNTAYLQMNSLKPEDTAVYSCAAGGNHYNPQYYHDYDKYDHWGQGTQVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 361 aa, Artificial Sequence
  holeFc of KiH10 VHH72_h1_E1D_R27L_E31D_Y32I_S56G_L97A-holeFc
DVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 110 - protein, 358 aa, Artificial Sequence
  VHH89_Q1D_Hum-knobFc
DVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 371 aa, Artificial Sequence
  holeFc of KiH15 VHH72_h1_E1D_R27L_E31D_Y32I_S56G_L97A-holeFc
DVQLVESGGGLVQPGGSLRLSCAASGLTFSDIAMGWFRQAPGKEREFVATISWSGGGTYYTDSVKGRFTISRDNAKNTVYLQMNSLRPEDTAVYYCAAAGAGTVVSEWDYDYDYWGQGTLVTVSSGGGGSGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 112 - protein, 368 aa, Artificial Sequence
  VHH89_Q1D-knobFc
DVQLQESGGGLVQPGGSLRLSCAASGFTLDYYAIGWFREVPGKEREGLSRIDSSDGSTYYADSVKGRFTISRDNTKNIVYLQMNNLKPEDTAVYYCATDPIIQGRNWYWTGWGQGTQVTVSSGGGGSGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 363 aa, Artificial Sequence
  VHH.xas51Hum-holeFc
DVQLVESGGGLVQPGGSLRLSCTASTPTIKNYAMGWFRQAPGKEREFLAAITFNSDATYYADSVKGRFTISRDNAKNTVYLQMNSLRPEDTAVYYCAAGGNHYNPQYYHDYDKYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 358 aa, Artificial Sequence
  VHH89Hum-knobFc
DVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 115 - protein, 359 aa, Artificial Sequence
  VHH3.117-hole Fc
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 363 aa, Artificial Sequence
  VHH.XAS.51hum-knobFc
DVQLVESGGGLVQPGGSLRLSCTASTPTIKNYAMGWFRQAPGKEREFLAAITFNSDATYYADSVKGRFTISRDNAKNTVYLQMNSLRPEDTAVYYCAAGGNHYNPQYYHDYDKYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 117 - protein, 359 aa, Artificial Sequence
  VHH3.1171-knobFc
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 118 - protein, 499 aa, Artificial Sequence
  VHH117_Q1D-15GS-VHH72_Q1D_S56A-10GS-Fc_EPKSCdel_LALA_Kdel
DVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSSGGGGSGGGGSGGGGSDVQLVESGGGLVQPGGSLRLSCAASGRTFSEYAMGWFRQAPGKEREFVATISWSGGATYYTDSVKGRFTISRDNAKNTVYLQMNSLRPEDTAVYYCAAAGLGTVVSEWDYDYDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 495 aa, Artificial Sequence
  VHHxas51Hum-10GS-hIgG1_EPKSCdel_YTE_K447del-10GS-VHH89Hum_D1E
DVQLVESGGGLVQPGGSLRLSCTASTPTIKNYAMGWFRQAPGKEREFLAAITFNSDATYYADSVKGRFTISRDNAKNTVYLQMNSLRPEDTAVYYCAAGGNHYNPQYYHDYDKYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSS

SEQ ID NO: 120 - protein, 495 aa, Artificial Sequence
  VHH89Hum-10GS-hIgG1_EPKSCdel_YTE_K447del-10GS-VHH83Hum
DVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDVQLVESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 121 - protein, 495 aa, Artificial Sequence
  VHH83Hum-10GS-hIgG1_EPKSCdel_YTE_K447del-10GS-VHH89Hum_D1E
DVQLVESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVQPGGSLRLSCAASGFTLDYYAIGWFRQAPGKEREGLSRIDSSDGSTYYADSVKGRFTISRDNAKNIVYLQMNSLKPEDTAVYYCATDPIIQGRNWYWTGWGQGTLVTVSS

SEQ ID NO: 122 - protein, 1255 aa, Artificial Sequence
  SARS-CoV-2 Wuhan WT Spike_del18
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 123 - protein, 1255 aa, Artificial Sequence; identical to SEQ ID NO: 122
  SARS-CoV-2 D614G Spike_del18

SEQ ID NO: 124 - protein, 1255 aa, Artificial Sequence
  SARS-CoV-2 Spike_del18_L452R/T478K
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 125 - protein, 1255 aa, Artificial Sequence
  SARS-CoV-2 Spike_del18_N501Y
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 126 - protein, 1255 aa, Artificial Sequence
  SARS-CoV-2 Spike_del18_N501Y/K417N/E484K
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 127 - protein, 1255 aa, Artificial Sequence
  SARS-CoV-2 Spike_del18_N501Y/K417T/E484K
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 128 - protein, 1255 aa, Artificial Sequence
  SARS-CoV-2 Spike_del18 (N501Y/K417N/E484K/L452R/T478K)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 129 - protein, 1252 aa, Artificial Sequence
  SARS-CoV-2 Omicron BA.1 Spike_del18
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFFDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 130 - protein, 1252 aa, Artificial Sequence
  SARS-CoV-2 Omicron BA.2 Spike_del18
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 131 - protein, 5 aa, Artificial Sequence
  CDR1 of VHH3.89 and VHH3_183 according to Kabat annotation
YYAIG

SEQ ID NO: 132 - protein, 5 aa, Artificial Sequence
  CDR1 of VHH3C_80 according to Kabat annotation
DYDVG

SEQ ID NO: 133 - protein, 17 aa, Artificial Sequence
  CDR2 of VHH3.89 and VHH3C_80 according to Kabat annotation
RIDSSDGSTYYADSVKG

SEQ ID NO: 134 - protein, 17 aa, Artificial Sequence
  CDR2 of VHH3_183 according to Kabat annotation
RIESSDGSTYYADSVKG

SEQ ID NO: 135 - protein, 12 aa, Artificial Sequence
  CDR3 of VHH3.89 according to Kabat annotation
DPIIQGRNWYWT

SEQ ID NO: 136 - protein, 12 aa, Artificial Sequence
  CDR3 of VHH3_183 according to Kabat annotation
DPIIQGSSWYWT

SEQ ID NO: 137 - protein, 12 aa, Artificial Sequence
  CDR3 of VHH3C_80 according to Kabat annotation
DPIIRGHNWYWT

SEQ ID NO: 138 - protein, 5 aa, Artificial Sequence
  consensus CDR1 of VHH3.89 family
  MISC_FEATURE (1)..(1): Xaa is D (Asp, Aspartic Acid) or Y (Tyr, Tryosine)
  MISC_FEATURE (3)..(3): Xaa is D (Asp, Aspartic Acid) or A (Ala, Alanine)
  MISC_FEATURE (4)..(4): Xaa is V (Val, Valine) or I (Ile, Isoleucine)
XYXXG

SEQ ID NO: 139 - protein, 17 aa, Artificial Sequence
  consensus CDR2 of VHH3.89 family
  MISC_FEATURE (3)..(3): Xaa is D (Asp, Aspartic Acid) or E (Glu, Glutamic Acid)
RIXSSDGSTYYADSVKG

SEQ ID NO: 140 - protein, 12 aa, Artificial Sequence
  consensus CDR3 of VHH3.89 family
  MISC_FEATURE (5)..(5): Xaa is R (Arg, Arginine) or Q (Gln, Glutamine)
  MISC_FEATURE (7)..(7): Xaa is R (Arg, Arginine), S (Ser, Serine) or H (His, Histidine)
  MISC_FEATURE (8)..(8): Xaa is N (Asn, Asparagine) or S (Ser, Serine)
DPIIXGXXWYWT

SEQ ID NO: 141 - protein, 5 aa, Artificial Sequence
  CDR1 according to Kabat of VHH4.1XAS51 and VHH4.1XAS43
NYAMG

SEQ ID NO: 142 - protein, 5 aa, Artificial Sequence
  CDR1 according to Kabat of VHH4.1XAS58
AFSIG

SEQ ID NO: 143 - protein, 6 aa, Artificial Sequence
  CDR1 according to Kabat of VHH4.1XAS31
SDYAVA

SEQ ID NO: 144 - protein, 17 aa, Artificial Sequence
  CDR2 according to Kabat of VHH72-5mut
TISWSGGGTYYTDSVKG

SEQ ID NO: 145 - protein, 17 aa, Artificial Sequence
  CDR2 according to Kabat of VHH4.1XAS51, VHH4.1XAS43, VHH4.1XAS58 and VHH4.1XAS31
AITFNSDATYYADSVKG

SEQ ID NO: 146 - protein, 18 aa, Artificial Sequence
  CDR3 according to Kabat of VHH4.1XAS51
GGNHYNPQYYHDYDKYDY

(19) United States
(12) Patent Application Publication
(10) Pub. No.: US 2024/0228596 A1
(43) Pub. Date: Jul. 11, 2024

(54) PAN-SPECIFIC CORONA VIRUS BINDERS

(71) Applicants: VIB VZW, Gent (BE); UNIVERSITEIT GENT, Gent (BE); EXEVIR BIO BV, Gent (BE)

(72) Inventors: Bert SCHEPENS, Gent (BE); Loes VAN SCHIE, Gent (BE); Hannah EECKHAUT, Oosterzele (BE); Wim NERINCKX, Astene (BE); Daria FIJALKOWSKA, Evergem (BE); Sieglinde DE CAE, Lennik (BE); Xavier SAELENS, Ieper (BE); Nico CALLEWAERT, Nevele (BE); Iebe Rossey, Gent (BE); Kenny ROOSE, Gent (BE)

(21) Appl. No.: 18/559,915

(22) PCT Filed: May 12, 2022
(86) PCT No.: PCT/EP2022/062980

§ 371 Date: Nov. 9, 2023

(30) Foreign Application Priority Data
May 12, 2021 (EP) 21173680.6

(51) Int. Cl. C07K 16/10; C07K 16/40

(52) U.S. Cl. CPC C07K 16/1003; C07K 16/1002; C07K 16/40; C07K 2317/31; C07K 2317/569; C07K 2317/76; C07K 2317/92

(57) ABSTRACT

Compositions and binding agents specifically binding the Spike protein of Corona viruses via at least two different binding sites and potently neutralizing coronaviruses, in particular sarbecoviruses, such as SARS-COV-1 and SARS-COV-2. The compositions or agents specifically bind to epitopes of the Receptor binding domain (RBD) of the Spike protein wherein both epitopes are conserved over multiple clades of the sarbecoviruses, providing broadly neutralizing pan-specific antibody-based compositions, thereby reducing viral escape. Application and uses of these agents and compositions are disclosed.

### FIELD

The invention relates to compositions and binding agents specifically binding the Spike protein of Corona viruses via at least two different binding sites and potently neutralizing coronaviruses, in particular sarbecoviruses, such as SARS-COV-1 and SARS-COV-2. The compositions or agents specifically bind to epitopes of the ACE2-receptor binding domain (RBD) of the Spike protein characterized in that both epitopes are conserved over multiple clades of the sarbecoviruses, providing broadly neutralizing pan-specific antibody-based compositions thereby reducing viral escape. Application and uses of these agents and compositions are further part of this invention.

### INTRODUCTION

Severe acute respiratory syndrome Coronavirus 2 (SARS-COV-2) is the causative agent of COVID-19, a disease that has rapidly spread world-wide with devastating consequences. SARS-COV-2 infections can be asymptomatic and mostly present with mild to moderately severe symptoms. However, in approximately 10% of patients, COVID-19 progresses to a more severe stage that is characterized by dyspnoea and hypoxemia, which may progress further to acute respiratory distress requiring often long-term intensive care and causing death in a proportion of patients. “Long-COVID” furthermore refers to long-term effects of COVID-19 infection, even when no SARS-COV-2 virus can be detected anymore. Most likely, the ongoing inflammation triggered by the innate recognition of the SARS-COV-2 virus, and possibly also by immune complexes with antibodies from an ineffective immune response, contributes to severe disease progression. Therapeutic options to suppress or even prevent (further) viral replication in the lower airways will likely find an important place in rescuing patients (elderly or other) that have contracted or re-contracted COVID-19. A particular type of therapeutic approach potentially relies on neutralizing antibodies, i.e. on passive antibody therapy/immunotherapy (egress of immunoglobulin from the systemic circulation into the broncho-alveolar space is augmented due to inflammation in the lower airways, systemic administration of a neutralizing antibody is thus feasible). Rujas et al. 2020 (doi: https://doi.org/10.1101/2020.10.15.341636) provide a good overview of antibodies binding to the spike protein (S) of SARS-COV-2 for which entries are available in the Protein Data Bank (PDB) or Electron Microscopy Data Bank (EMDB), and provide some new antibodies, some of which (antibodies 46 and 52) with a binding site shifting somewhat away from the receptor binding motif and potentially destabilizing the spike protein.

Similar to the severe acute respiratory syndrome virus (SARS) caused by SARS-COV-1, SARS-COV-2 uses the angiotensin converting enzyme 2 (ACE2) as a receptor for entry into human cells. SARS-COV-2 binds ACE2 with a higher affinity than SARS-COV-1. Cross-reactivity of antibodies to the S-domain of SARS-CoV to SARS-COV-2 is described by Bates et al. 2021 (Cell Rep 34:108737).

The receptor-binding motif (RBM) of the Corona virus Spike protein that interacts with the human ACE2 receptor provides for an immunogenic region to develop neutralizing human antibodies, though it is also one of the hotspot where SARS-COV-2 variant mutations arise, as demonstrated by a number of spreading SARS-COV-2 variants, such as the B.1.1.7 variant that emerged in the United Kingdom and the 501Y.V2 variant that was found in South Africa, that are capable of reducing neutralization potency of some monoclonal antibodies that bind the RBM region and, worryingly, even of human convalescent plasma (Leung, et al. 2021, Euro Surveill. 26, 2002106). The monoclonal antibodies casirivimab and imdevimab (Regeneron) and bamlanivimab (Lilly), have received emergency use authorization from US FDA on Nov. 9 2020. SARS-COV-2 variants B.1.351 (South Africa; includes variants in the RBD K417N, E484K, N501Y) and B.1.1.248 (Brazil; includes variants in the RBD K417T, E484K, and N501Y) were very recently reported to be partially resistant to casirivimab and to be fully resistant to bamlanivimab (Hoffmann et al. 2021, doi: https://doi.org/10.1101/2021.02.11.430787), amply demonstrating the need for additional therapeutic options. As a result the FDA revoked the emergency use authorization of bamlanivimab as a monotherapy on Apr. 16 2021. The emergence of variants urges the need to develop neutralizing antibodies that bind to epitopes that are under reduced selection pressure by the human antibody response. For this, single-domain antibodies are particularly suited. With their small size, some single-domain antibodies can reach sites in spike that are more difficult to access by conventional antibodies. Single domain antibody/nanobody-format neutralizers of both SARS-COV-1 and -2 have been reported such as VHH72 by Wrapp et al. 2020 (Cell 184:1004-1015). An additional way of reducing the chance of viral escape from immunity is by merging different VHHs into biparatopic constructs (FIG. 1, adapted from Saelens and Schepens, 2021, Science 371 (6530), 681-682). For instance, Koenig et al. (2021, Science 371, eabe6230) showed that tandem repeats of two neutralizing VHHs that bind non-overlapping epitopes strongly reduced the chance that mutant viruses that escape neutralization were selected in vitro. Wu et al. 2021 (BioRxiv doi: https://doi.org/10.1101/2021.02.08.429275) reported a series of SARS-COV-2 neutralizing nanobodies claiming that a bispecific nanobody format increases potency in the setting of intranasal administration. To reduce the likelihood of emergence of mutant coronaviruses escaping neutralization, there is a need for pan-specific antibodies that bind to several epitope regions which provide a mixture of binding sites that are less prone to viral mutation.

### SUMMARY OF THE INVENTION

The invention relates to pan-specific binding agents capable of binding the sarbecovirus spike protein Receptor Binding Domain (SPRBD) via at least two binding sites that are both conserved among sarbecoviruses.

In a first aspect, the invention relates to a composition containing one or more binding agents, which specifically bind the Corona virus Spike protein RBD, wherein the one or more agents comprise one or more first immunoglobulin single variable domains (ISVDs) binding to the amino acid residues Y369, F377, and K378 of the SARS-COV-2 spike protein as defined by SEQ ID NO:1, and one or more second ISVDs binding to at least one or more of the amino acids T393, N394, V395, or Y396 of the SARS-COV-2 spike protein as defined by SEQ ID NO:1. In fact said binding sites of said first and second ISVDs may also be defined as the minimal residues needed for a binding agent or binding domain to specifically interact with VHH72 (Wrap et al. 2020; Cell 184:1004-1015; PCT/EP2021/052885) and VHH3.117 (as shown herein, and EP21166835.5 and PCT/EP2022/052919), respectively. Said binding sites of said first and second ISVDs provide for a dual binding region each of which separately allow for neutralization of SARS-COV-1 and SARS-COV-2 viruses, and providing for a binding region on the RBD domain of the corona virus spike protein that is conserved among the sarbecoviruses, and thus less prone to mutation and escape from neutralization.

The invention also relates to a binding agent comprising one or more first ISVDs binding to the amino acid residues Y369, F377, and K378 of the SARS-COV-2 spike protein as depicted in SEQ ID NO:1, and one or more second ISVDs binding to at least one or more of the residues T393, N394, V395, or Y396 of the SARS-COV-2 spike protein as depicted in SEQ ID NO:1.

In a further aspect, the invention relates to an isolated nucleic acid molecule encoding the binding agents of the invention, as well as to a recombinant vector comprising said nucleic acid molecule.

Another aspect relates to a pharmaceutical composition, comprising the composition, the binding agent, the isolated nucleic acid and/or the recombinant vector according to the invention as described hereinabove, and said pharmaceutical composition optionally comprising a diluent, carrier or excipient.

The invention likewise relates to the above-described (pharmaceutical) composition, binding agent, isolated nucleic acid and/or a recombinant vector, for use as a medicament. The invention likewise relates to the above-described (pharmaceutical) composition, binding agent, isolated nucleic acid and/or a recombinant vector, for use in prophylactic treatment of a subject. The invention likewise relates to the above-described (pharmaceutical) composition, binding agent, isolated nucleic acid and/or a recombinant vector, for use in the treatment of a coronavirus infection, more specifically a sarbecovirus infection, more specifically in treatment of SARS-COV-1 or SARS-COV-2 infection. The invention likewise relates to the above-described (pharmaceutical) composition, binding agent, isolated nucleic acid and/or a recombinant vector, for use in passive immunisation of a subject.

### DESCRIPTION OF THE FIGURES

The drawings described are only schematic and are non-limiting. In the drawings, the size of some of the elements may be exaggerated and not drawn on scale for illustrative purposes.

FIG. 1. Bispecific antibody compositions targeting the Spike protein to neutralize SARS-COV-2. The spike protein of SARS-COV-2 contains the RBD that binds to ACE2 on host cells, leading to cell entry. RBD-ACE2 binding can be prevented by single-domain antibodies, or VHHs. Combined treatment with VHH1 and VHH2, which bind nonoverlapping regions of the RBD, prevents infection until escape mutants arise. This can be overcome when the VHHs are covalently linked in a bispecific molecule. (adapted from Saelens and Schepens, Science 371 (6530), 681-682). ACE2, angiotensin-converting enzyme 2; RBD, receptor-binding domain; SARS-COV-2, severe acute respiratory syndrome coronavirus 2.

FIG. 2. Dose-dependent inhibition of VHH72 binding to SARS-COV-2 RBD by VHHs from different families. Competition Alphascreen with avi-tagged biotinylated SARS-COV-2 RBD (0.5 nM final) and Flag-tagged VHH72 h1 S56A (0.6 nM). VHHs belonging to the same (super) family are indicated in boxes.

FIG. 3. The epitopes of VHH72, VHH3.38, VHH3.83 and VHH3.55 based on the deep mutational scanning. The profile of the RBD amino acid positions involved in the binding of VHH72_h1_S56A, VHH3.38, VHH3.55 and VHH3.83 as determined by deep mutational scanning (black lines) overlaps among the VHHs and with the VHH72 epitope on the SARS-COV-2 RBD based on FastContact and modeling (orange bars).

FIG. 4. Kinetics of VHH3.117 binding to RBD. (A) Comparison of the off rates of VHH3.117 (“VHH3_117”), VHH3.42 (“VHH3_042”) and VHH72_h1_S56A (“VHH72”) as measured by BLI at a single concentration (200 nM) to monomeric human Fc-fused SARS-COV-2_RBD-SD1 immobilized on anti-human IgG Fc capture (AHC) biosensors (ForteBio). Each graph shows one of the duplicate measurements. (B) Binding kinetics of VHH3.117 to monomeric human Fc-fused SARS-COV-2_RBDSD1 immobilized on anti-human IgG Fc capture (AHC) biosensors (FortéBio), in replicate, at concentrations of 200 to 3.13 nM (2-fold dilution series).

FIG. 5. VHH3.42 and VHH3.117 do not compete with VHH72 for the binding of RBD. (A) VHH3.42 and VHH3.117 can bind to monomeric SARS-COV-2 RBD captured by VHH72-Fc. The graph shows the average (n=2±variation) binding (OD at 450 nm) of the VHHs and an irrelevant GFP binding VHH (GBP) at 0.5 μg/ml to RBD that was captured by coated VHH72-Fc. PBS and VHH72_h1_S56A (“VHH72”) at 10 μg/ml were included as reference. (B) In this BLI competition experiment, VHH72-Fc was loaded on anti-human Fc biosensor tip and subsequently dipped into a solution containing mouse IgG2a Fc-fused SARS-COV-2-RBD-SD1 (Sino Biological) until saturation was achieved. Next, the tips were dipped into a solution containing VHH72_h1_S56A (“VHH72”), VHH3.42 (“VHH3_42”), VHH3.117 5 (“VHH3_117”) or no VHH (“buffer”). VHHs that compete with VHH72 for the binding of RBD (such as VHH72 itself) displace the captured RBD-muFc from the VHH72-Fc coated tips and will hence lower the BLI signal over time. VHH3.42 and VHH3.172 bind to VHH72-Fc captured RBD, resulting in an increased BLI signal. The graph shows the BLI signal over time starting from the moment the tips were dipped in the solution containing the indicated VHHs.

FIG. 6. VHH3.42, VHH3.92 and VHH3.117 do not interfere with the binding of RBD to recombinant ACE2. The graph shows the AlphaLISA signal that is detected upon binding of biotinylated RBD to recombinant ACE2 in the presence of dilution series of VHH3.42, VHH3.42 and VHH3.117. A control VHH targeting an irrelevant protein was used as negative control (ctrl VHH). VHH72_h1_S56A 30 (“VHH72”) and the related VHH3.115 that both prevent binding of RBD to ACE2 were used as positive controls.

FIG. 7. VHH3.42, VHH3.92 and VHH3.117 do not prevent binding of RBD to ACE-2. (A-C) VHH3.42, VHH3.92 and VHH3.117 do not prevent binding of RBD to Vero E6 cells. (A) RBD-Fc binding to a E6 cell that endogenously expresses ACE2; flow cytometric analysis of binding of RBD (0.4 μg/ml) that was pre-incubated with VHH3.42 or VHH3.117 (each at 1 μg/ml) to Vero E6 cells. As controls Vero E6 cells not treated with RBD (noRBD) and Vero E6 cells stained with RBD-muFc that was pre-incubated with PBS or an irrelevant control GFP targeting VHH (ctrl VHH) were used. VHH72_h1_S56A was used as reference. The bars represent one single analysis per VHH. The controls, PBS and noRBD were tested in duplicate. Binding of RBD-muFc was detected by an AF647 conjugated anti-mouse IgG antibody. (B) Flow cytometric analysis of binding of RBD (0.4 μg/ml) that was pre-incubated with a dilution series of VHH3.92 or VHH3.117 to Vero E6 cells. As controls Vero E6 cells not treated with RBD (noRBD) and Vero E6 cells stained with RBD-muFc that was pre-incubated with PBS or an irrelevant control GFP targeting VHH (ctrl VHH) were used. VHH3.115 (a VHH related to VHH72) was used as reference. Binding of RBD-muFc was detected by an AF647 conjugated anti-mouse IgG antibody. The graph shows the % RBD-muFc positive Vero E6 cells (n=1). (C) VHH3.117 does not prevent binding of human ACE2 fused to a human Fc to yeast cells expressing the SARS-COV-2 RBD at their surface. Histograms showing the binding of ACE2-Fc that was pre-incubated with VHH72 or 15 VHH3.117 (at 10, 1, 0.1, 0.01 or 0 μg/ml). Binding of ACE2-Fc was detected using an AF594 conjugated anti-human IgG antibody.

FIG. 8. VHH3.117 recognizes the RBD of a diverse range of clade 1, 2 and 3 Sarbecoviruses. (A) Flowcytometric analysis of the binding of VHH3.117 to the indicated RBDs at 100 (left bar per datapoint on the X-axis), 1 (middle bar per datapoint on the X-axis) and 0.01 μg/ml (right bar per datapoint on the X-axis). (B) PBS was used as negative control and VHH72_h1_S56A (“VHH72”) was used as reference. The graphs show for the indicated RBD variants the ratio of the MFI of AF647 conjugated anti-mouse IgG antibody used to detect VHHs bound the Saccharomyces cerevisiae cells that express RBD (FITC conjugated anti-myc tag antibody positive) over that of cells that do not express RBD (FITC conjugated anti-myc tag antibody negative).

FIG. 9. Outlining of the VHH3.117 epitope identified by deep mutational scanning. (A) Indication of the RBD amino acid positions for which changes can significantly affect the binding of VHH72_h1_S56A (“VHH72 escape”) and VHH3.117 (“VHH3.117 escape”) as identified by deep mutational scanning using 2 independent libraries. The SARS-COV-2 RBD amino acid sequence is shown (SEQ ID NO:99, corresponding to the residues 381-531 of SEQ ID NO:1 spike protein SARS-COV-2) in the upper and lower line. In the upper line the amino acid positions at which mutations result in reduced VHH72_h1_S56A binding to the RBD of SARS-COV-2 displayed on the surface of S. cerevisiae cells, are underlined and in bold. In the lower line the amino acids positions at which mutations result in reduced binding of VHH3.117 to the RBD of SARS-COV-2 displayed on the surface of S. cerevisiae cells, are underlined and in bold. (B) Top left panel: Surface representation of the SARS-COV-2 RBD (light grey) with the amino acid positions for which a change, as identified by deep mutational scanning, is associated with reduced binding of VHH3.117 are indicated in dark grey. Top right panel: cartoon representation of the SARS-COV-2 RBD (light grey). The amino acid positions for which certain substitutions are associated with reduced VHH3.117 binding and that are surface exposed are indicated in dark red and shown as sticks in the cartoon representation. Bottom left and right panels: amino acid positions for which some substitutions are associated with reduced VHH3.117 binding but are not exposed to the RBD surface are indicated. The bottom left cartoon shows the C336-C361 and C391-C525 disulfide bonds. The bottom right panel illustrates that the aromatic side chains of Y365 and F392 are oriented inwards into the RBD core.

FIG. 10. The location of the identified VHH3.117 epitope is in line with the ability of VHH3.117 to cross-neutralize SARS-COV-2 and SARS-COV-1 viruses. (A) The VHH3.117 binding site is highly conserved among the SARS-COV-2 RBD sequences in the GISAID database. Surface representations of the SARS-COV-2 RBD (white) showing the degree of conservation. The white to black gradient represents the most to the least conserved positions. Amino acids that are substituted in emerging variants of concern (K417, L452, E484 and N501) or in variants of interest (S477), as well as in N439 are pointed out by arrows. (B) The amino acid sequence of SARS-COV-2 RBD (SEQ ID NO: 100, spike protein amino acid positions 333-516 of Wuhan-Hu-1 isolate) is shown with all missense mutations, detected at least once in 440,769 SARS-COV-2 genomes analyzed (available in GISAID on Feb. 12, 2021), depicted above each residue. Variants are ordered vertically at each position, according to frequency represented by the number of observed cases. Amino acids that are substituted in emerging variants of concern (K417, L452, E484 and N501) or in variants of interest (S477) are indicated by asterisk. The N439 position that is frequently substituted is also indicated. The amino acids for which substitutions were associated with reduced binding of VHH3.117 as determined by deep mutational scanning are indicated in boxes. The VHH3.117 epitope is not accessible on intact spike proteins. The VHH3.117 binding site is not accessible on the RBD in down- or in up-conformation. Shown is the SARS-COV-2 spike trimer (PDB: 6VSB, white) with 1 RBD in up-conformation and 2 RBDs in down conformation. The VHH3.117 binding region is marked in dark grey and indicated with one arrow that points to the RBD in the up position and another arrow that points to one of the RBDs in the down position. Inset: the VHH3.117 binding site on the RBD in up conformation is partially occluded by an NTD of an adjacent spike protomer.

FIG. 11. VHH3.117 and VHH72 neutralize SARS-COV-2 in a co-operative way. (A) Surface presentation of the SARS-COV-2 RBD (grey) in complex with VHH72 (black). The VHH3.117 epitope as deduced from Deep Mutational Scanning is indicated in red on the left side. (B) VSV-ΔG virus pseudotyped with the SARS-COV-2 spike is neutralized by the VHH72_h1-S56A/VHH3.117 cocktail more efficiently than by VHH72 and VHH3.117 individually. Serial dilution of 32 times the EC50 (VSV-ΔG spike SARS-COV-2 neutralization) of VHH72 or VHH3.117 or 1:1 mixes of both VHHs at half this concentration were mixed with VSV-ΔG spike (SARS-COV-2), incubated at 37° C. for 30 minutes and added to Vero E6 cells. Sixteen hours after infection, cell lysates were prepared and tested for GFP fluorescence. The graph shows the mean MFI of triplicate samples (n=3±SD).

FIG. 12. Surface representation of SARS-COV-2 RBD and anti-RBD VHHs. (A) Surface presentation of the SARS-COV-2 RBD (grey) in complex with VHH72 (in green ribbon representation). The VHH3.117 epitope is indicated in red on the left side, and the area likely covered by VHH3.117 is indicated by the red pane. (B) the contact region of VHH3.117 is shown in red in a surface representation of the RBD positioned with the VHH72 binding on the opposite side in the back. (C) similar to the VHH72 binding site, VHH3.83 binding (indicated by the orange pane) covers an area of the RBD different from the VHH3.117 binding region (in red).

FIG. 13. Bi-specific, bivalent and tetravalent constructs of RBD-binding VHHs as Fc fusion proteins. (A) Bivalent or bispecific VHH constructs are made via fusion with a suitable linker, wherein A and B are different monovalent VHH building blocks. Further multivalent linking is also envisaged with another A, B, or C VHH monovalent building block. (B) Bivalent or (C) bispecific Fc fusion constructs can be made by fusion of VHH building blocks (A and/or B) to a human Fc domain, for instance from an IgG, preferably from an IgG1, wherein said Fc tail may be construed via knob-into-hole technology (C). (D) Tetravalent constructs using bispecific VHHs as Fc-fusions. (E) Tetravalent bispecific VHH_A-Fc-VHH_B constructs in which a first VHH is fused to the N-terminus of the Fc via a linker and hinge and were a second VHH, recognizing a different epitope is fused to the C-terminus of a human Fc via a linker.

FIG. 14: Bivalent constructs of head-to-tail fused VHHs potently neutralize VSV-ΔG pseudotyped with SARS-COV-2 spikes. (A) Monospecific constructs comprising head-to-tail fused VHH72-h1-E1D-S56A copies with G4S linkers of various lengths neutralize VSV-delG pseudotyped with the SARS-COV-2 spikes. The graph shows the GFP fluorescence intensity of dilutions series (n=3±SEM) of B001, B002, B003 (encoded by pX-B1, pXB2 and pX-B3, provided in SEQ ID NO: 73-75, resp.), VHH3.117 and VHH3.83 (as depicted in SEQ ID NO:22 and SEQ ID NO:6, resp.) each normalized to the highest and lowest GFP signal of each dilution series. (B) Bispecific constructs comprising head-to-tail fused VHH3.117 and VHH3.83 or the VHH3.83-N85E variant (encoded by pX-B7, pX-B8, and pX-B11, depicted as B007, B008, and B011, and as in SEQ ID NO:79, 80, 83, resp.) neutralize VSV-delG pseudotyped with the SARS-COV-2 spike protein. The graph shows the GFP fluorescence intensity of dilutions series of the indicated bivalent and monovalent VHHs (n=3±SEM), each normalized to the highest and lowest GFP signal of each dilution series.

FIG. 15. A bispecific VHH construct comprising head-to-tail fused VHH3.117 and VHH3.82 binds to the RBD of a broad range of sarbecoviruses. The graphs show the binding (OD at 450 nm) of dilution series of VHH3.117, VHH3.92, VHH3.83 and B007 (GS-VHH3-117-hc_(G4S)6_VHH3-83-hc_His8) to coated yeast cells expressing the RBD of the indicated sarbecoviruses at their surface. GBP, a GFP binding VHH was used as negative control.

FIG. 16: Bispecific constructs of head-to-tail fused VHHs potently neutralize VSV-ΔG pseudotyped with SARS-COV-2 spikes. Bispecific constructs comprising head-to-tail fused VHH3.117 and VHH3.83 (encoded by pX-B7, pX-B9 and pX-B10, and depicted in SEQ ID NOs:79, 81, and 82, resp.) or VHH72-h1-E1D-S56A (encoded by pX-B4 and pX-B5, depicted as in SEQ ID NO: 76-77, resp.) with G4S linkers with various lengths neutralize VSV-delG pseudotyped with the SARS-COV-2 spikes. The graph shows the GFP fluorescence intensity of dilutions series (n=3±SEM) of the indicated bispecific constructs, VHH3.117 and VHH3.83-hc (‘M6’; as depicted in SEQ ID NO: 7) each normalized to a non-infected and infected PBS treated sample included in each dilution series. The table below provides the EC50 values for each of the samples in μg/mL.

FIG. 17. VHH3.89 does not compete with VHH72, S309 or CB6 but does compete with VHH3.177 for binding to the SARS-COV-2 RBD. (A) Binding of VHH3.89 to RBD pre-bound by well-characterized antibodies. The graphs show the average binding (OD at 450 nm) and variation (n=2) of dilution series of VHH3.92 that is related to VHH3.117 (left panel) or VHH3.89 (right panel) to RBD-SD1 fused to monovalent human Fc (RBD-SD1-monoFc) that was either directly coated on an ELISA plate or captured by coated S309, CB6, D72-53 and VHH3.117 (without HA-tag). RBD that was captured by palivizumab (Synagis), an antibody directed against the RSV F protein was used as negative control. Binding of HA-tagged VHH3.92 and VHH3.89 was detected by an anti-HA tag antibody. (B) Surface representation of the SARS-COV-2 RBD captured by S309, CB6 and VHH72 shown as meshes. The black and white coloring of the RBD surface respectively indicate amino acids that are different or identical between SARS-COV-1 and 2. (C) VHH3.117 binds to a concave site at the side of the RBD. The black coloring on the RBD surface representation indicates the amino acid positions at which substitutions are associated with reduced binding of VHH3.117 as determined by deep mutational scanning based on yeast surface display of RBD mutants.

FIG. 18. VHH3.89 does not prevent binding of RBD to ACE-2. Flow cytometric analysis of binding of RBD-muFc (0.4 μg/ml) that was pre-incubated with a dilution series of VHH3.89 or VHH3.117 to Vero E6 cells. Vero E6 cells not treated with RBD (noRBD) and Vero E6 cells stained with RBD-muFc that was pre-incubated with PBS or an irrelevant control GFP targeting VHH (ctrl VHH) were used as controls. VHH3.115, an VHH related to VHH72 and known to block the binding of RBD to ACE2, was used as control. Binding of RBD-muFc was detected by an AF647 conjugated anti-mouse IgG antibody. The graph shows the binding (n=1) of RBD-muFc (MFI of AF647) to Vero E6 cells.

FIG. 19. VHH3.89 neutralizes VSV-ΔG pseudotyped with the SARS-COV-2 or SARS-COV-1 spikes. (A) VHH3.89, neutralizes VSV-delG pseudotyped with the SARS-COV-2 spikes. Neutralization of SARS-COV-2 pseudotyped VSV (VSV-ΔG spike SARS-COV-2) by purified VHH3.89, VHH3.117 and VHH3.92 and VHH3.83. The graph shows the GFP fluorescence intensity of quadruplicate dilutions series (n=4±SEM), each normalized to a non-infected and infected PBS treated sample included in each dilution series. The GFP binding VHH, GBP, was used as negative control (B) VHH3.89 neutralizes VSV-delG pseudotyped with the SARS-COV-1 spike protein. Neutralization of SARS-COV-1 pseudotyped VSV (VSV-ΔG spike SARS-COV-2) by crude E. coli periplasmic extracts containing VHH3.89, VHH3.117, VHH3.92 or VHH3.83. The graph shows the GFP fluorescence intensity normalized to a non-infected sample and infected PBS treated sample. A periplasmic extract that did not contain an SARS-COV-2 spike protein binding VHH (PE control) was used as negative control.

FIG. 20. VHH3.89 recognizes the RBD of a diverse range of sarbecoviruses. (A) Cladogram (UPGMA method) based on the RBD of SARS-COV-1-related (clade 1a), SARS-COV-2-related (clade1b) and clade 2 and clade 3 Bat SARS-related Sarbecoviruses. The arrows indicate the viruses of which the RBD was included in the binding analysis (B) Surface representation of the SARS-COV-2 RBD displaying the degree of amino acid conservation among the tested sarbecoviruses as colored from red (most conserved) to blue (least conserved). Conservation analysis and visualization was done by Scop3D (Vermeire et al, 2015 Proteomics, 15(8): 1448-52) and PyMol (DeLano, 2002). (C) Flow cytometric analysis of the binding of dilution series of VHH3.117 and VHH3.89 to Saccharomyces cerevisiae cells that display the RBD of the indicated Sarbecoviruses at their surface. The graphs show for the tested RBD variants the ratio of the MFI of AF647 conjugated anti-mouse IgG antibody used to detect VHHs bound to the cells that express RBD (FITC conjugated anti-myc tag antibody positive) over that of cells that do not express RBD (FITC conjugated anti-myc tag antibody negative). (D) VHH3.89 efficiently binds to the RBD of all clade 1 and 2 sarbecoviruses in a yeast cell ELISA. The graphs show the binding (OD at 450 nm) of dilution series of VHH3.89 and VHH3.117 to coated yeast cells expressing the RBD of the indicated sarbecoviruses at their surface.

FIG. 21. Escape analysis based on deep mutational scanning of the RBD via yeast surface display. (A) Escape of RBD variants from binding by head-to-tail fused VHHs respectively targeting epitope 1 and 2 is very limited. The panels show for the indicated VHH constructs/composition for each RBD AA position the cumulative fraction (between 0 and 1 for each AA) of all AA substitutions at that position that escaped from VHH binding. (B) Limited options to escape from head-to-tail fused VHHs respectively targeting epitope 1 and 2. The graphs show per position the identity and fraction (0-1) of yeast cells expressing an RBD variant with the indicated substitution that can escape from binding by VHH3.83, VHH3.117, a composition of both VHHs and the head-to-tail fused VHH3.117 and VHH3.83 (B008).

FIG. 22. A cocktail/composition or head-to-tail fusion of an epitope 1 binding VHH and an epitope 2 binding VHHs strongly restricts the number of RBD positions at which escape can occur. (A) The upper sequence represents the wild type RBD sequence (SEQ ID NO:101, corresponding to the residues 331-531 of SEQ ID NO: 1 spike protein SARS-COV-2). The indicated amino acids below represent for each VHH/VHH construct/VHH composition as indicated the amino acids at which mutations significantly escaped from VHH binding. (B) Surface representation of the RBD, with indicated in black, the AA positions at which mutations significantly escaped from binding of VHH3.117, VHH3.83, a composition of VHH3.117 and VHH3.83 and the head-to-tail fused VHH3.117 and VHH3.83 (B8).

FIG. 23. B008 retains neutralizing activity against SARS-COV-2 variants that escape neutralization by monovalent VHHs as tested by neutralization assays using VSV v particles pseudotyped with the indicated spike variants. Neutralization assays were performed using VSV particles pseudotyped with either WT (Wuhan) SARS-COV-2 spike protein (A) or spike proteins containing the K378N substitution (B) or the Y396H substitution (C). The graphs show the mean GFP fluorescence intensity of duplicate dilutions (N=2±variation) each normalized to mock infected and infected untreated controls that were included in each dilution series. (D) The graph shows for each VHH construct indicated the IC50 values that were calculated from the duplicate dilution series via non-linear regression curve fitting (log(inhibitor) vs. normalized response—Variable slope).

FIG. 24. B008 potently neutralizes the SARS-COV-2 variants of concern Alpha, Beta, Gamma and Delta as tested by neutralization assays using VSV particles pseudotyped with the spikes of the indicated variants. Neutralization assays were performed using VSV particle pseudotyped with either WT (Wuhan) SARS-COV-2 spike protein (A) or spike proteins in which the RBD mutations of the i SARS-CoV-2 variants of concern Alpha (B), Beta (C), Gamma (E) and Delta (D) were introduced. In addition, a neutralization assay was performed using VSV particles pseudotyped with spike proteins in which the mutations of the Alpha, Beta, Delta and Gamma variant were combined (N501Y, K478N, E484K, L452R and T478K) (F). The graphs show the mean GFP fluorescence intensity of dilutions (N=2±variation for D72-53 and B008 and N=3±SEM for VHH3.83-Fc and VHH3.117-Fc) each normalized to mock infected and infected untreated controls that were included in each dilution series.

FIG. 25. Knob-into-hole VHH-Fc constructs containing VHH3.83 and VHH3.117 potently neutralize SARS-COV-2 variants that escape from neutralization by the individual monovalent VHHs. Neutralization assays were performed using VSV particle pseudotyped with either WT (Wuhan) SARS-CoV-2 spike protein (A) or spike proteins in which the substitutions K378N (B), Y396Y (C) or K378N+Y396H (D) were introduced. The graphs show the mean GFP fluorescence intensity of duplicate dilution series (N=2±variation) each normalized to mock infected and infected untreated controls that were included in each dilution series. (E) The graph shows for each VHH construct the IC50 values that were calculated from the duplicate dilution series via non-linear regression curve fitting (log(inhibitor) vs. normalized response—Variable slope). (F) Surface representation of the RBD with the K378 and Y396 AA indicated by arrows and colored in black.

FIG. 26. Knob-into-hole VHH-Fc constructs containing epitope 1 and 2 targeting VHHs potently neutralize the SARS-COV-2 BA.2 Omicron variant. Neutralization assays were performed using VSV particles pseudotyped with either WT (Wuhan) SARS-COV-2 spike proteins (A) or SARS-COV-2 Omicron BA.2 spike proteins (B). The graphs show the mean GFP fluorescence intensity of triplicate dilution series (N=3±SEM) each normalized to mock infected and infected untreated controls that were included in each dilution series. (C) The graph shows for each VHH construct the mean IC50 values (N=3±SD) that were calculated from the dilution series via non-linear regression curve fitting (log(inhibitor) vs. normalized response—Variable slope). (D) Surface representation of the RBD with the Omicron BA.2 mutations colored in black.

FIG. 27. Knob-into-hole VHH-Fc constructs containing epitope 1 and 2 targeting VHHs recognize the RBD of clade 1, clade 2 and clade 3 Sarbecoviruses with highly similar affinities. The graphs show the binding (OD at 450 nm) of dilution series of VHH3.83-Fc, VHH3.117-Fc, KiH19, S309, CB6 and the Respiratory Syncytial Virus-specific control antibody palivizumab to coated yeast cells expressing the RBD of the indicated Sarbecoviruses of clade1 (SARS-Cov-2, GD-pangolin, RatG13, SARS-Cov-1, WIV1/6, LyRa) clade 2 (Rp3, HKU1, Rf1, ZXC21) and clade 3 (BM48-1) at their surface. Yeast cells not expressing any RBD (empty) were used as negative controls.

FIG. 28. Knob-into-hole VHH-Fc constructs containing epitope 1 and 2 targeting VHHs potently neutralize authentic SARS-COV-2 virus. (A) The graph shows the average number of plaques counted for triplicate dilutions series of the indicated antibodies (N=3±SEM). The plaque numbers were normalized to that of untreated infected control samples. (B) The graph shows for each VHH construct the mean IC50 values of two independent plaque reduction assays that were each performed in triplicate. The IC50 was calculated from the triplicate dilution series via non-linear regression curve fitting (log(inhibitor) vs. normalized response—Variable slope).

FIG. 29. Knob-into-hole VHH-Fc constructs comprising the epitope 1 binding VHH: VHH72-5mut and the epitope 2 binding VHH: VHH3.89 efficiently neutralize WT and SARS-COV-2 variants that are resistant to neutralization by VHH72-5mut. Neutralization assays were performed using VSV particle pseudotyped with either WT (Wuhan) SARS-COV-2 spike proteins (A) or variant spike proteins comprising the Y508H (B) or the S375F (C) substitution. The graphs show the mean GFP fluorescence intensity of duplicate dilution series (N=2±variation) each normalized to mock infected and infected untreated controls that were included in each dilution series. (D) The graph shows for each VHH construct the IC50 values that were calculated from the duplicate dilution series via non-linear regression curve fitting (log(inhibitor) vs. normalized response—Variable slope). (E) Surface representation of the SARS-COV-2 RBD with the Y508 and S375 AA indicated by arrows and colored in black.

FIG. 30. Knob-into-hole VHH-Fc constructs comprising the epitope 1 binding VHH: VHH72-5mut and the epitope 2 binding VHH: VHH3.89 recognize the RBD of all tested clade 1, clade 2 and clade 3 Sarbecoviruses. The graphs show the binding (OD at 450 nm) of dilution series of VHH72-5mut-Fc (A), VHH3.89-Fc (B) and KiH10 (C) to coated yeast cells expressing the RBD of the indicated Sarbecoviruses of clade1 (GD-pangolin, RatG13, SARS-Cov-1, WIV1/6, LyRa) clade 2 (Rp3, HKU1, Rf1, ZXC21) and clade 3 (BM48-1) at their surface. Yeast cells not expressing any RBD (empty) were used as negative controls.

FIG. 31. Neutralization of VSV particles pseudotyped with the SARS-COV-2 spike by VHH-VHH-Fc constructs and Knob-into-hole VHH-Fc constructs with formatted hinge and Fc. (A) Fc fusions of head-to-tail fused VHHs that respectively recognize epitope 1 and 2 can potently neutralize VSV particles pseudotyped with SARS-COV-2 spikes. The graphs show the mean GFP fluorescence intensity of triplicate dilution series (N=3±SEM) each normalized to mock infected and infected untreated controls that were included in each dilution series. (B) Formatting the hinge and Fc does not reduce the neutralizing activity of knob-into-hole VHH-Fc constructs comprising epitope 1- and epitope 2 binding VHHs. The graphs show the mean GFP fluorescence intensity of triplicate dilution series (N=3±SEM) each normalized to mock infected and infected untreated controls that were included in each dilution series. (C) The graph shows for each VHH construct the IC50 values that were calculated from the duplicate dilution series via non-linear regression curve fitting (log(inhibitor) vs. normalized response-Variable slope).

FIG. 32. Fc fusions of head-to-tail fused VHHs that respectively recognize epitope 1 and epitope 2 recognize the RBD of clade 1, clade 2 and clade 3 Sarbecoviruses. The graphs show the binding (OD at 450 nm) of dilution series of 117-72(S56A)-Fc (A) and palivizumab (B) to coated yeast cells expressing the RBD of the indicated Sarbecoviruses of clade1 (SARS-COV-2, GD-pangolin, RatG13, SARS-Cov-1, WIV1/6, LyRa) clade 2 (Rp3, HKU1, Rf1, ZXC21) and clade 3 (BM48-1) at their surface. Yeast cells not expressing any RBD (empty) were used as negative controls.

FIG. 33. Various knob-into-hole VHH-Fc constructs comprising epitope 1- and epitope 2-binding VHHs consistently neutralize WT SARS-COV-2 and SARS-COV-2 Omicron BA.1 and BA.2 variants. Neutralization assays were performed using VSV particles pseudotyped with either SARS-COV-2 614G spike proteins (A) or spike proteins of the Omicron BA.1 (B) or BA.2 (C) variants. The graphs show the mean GFP fluorescence intensity single dilution series each normalized to mock infected and infected untreated controls that were included in each dilution series.

FIG. 34. Bispecific VHHa-Fc-VHHb fusions comprising two VHHs that respectively target epitope 1 and 2 potently neutralize SARS-COV-2 WT and the BA.1 Omicron variant. Neutralization assays were performed using VSV particles pseudotyped with either 614G spike proteins (A) or spike proteins of the Omicron variant BA.1 (B). The graphs show the mean GFP fluorescence intensity of quadruplicate dilution series (N=4±SEM) each normalized to mock infected and infected untreated controls that were included in each dilution series.

FIG. 35. Bispecific VHHa-Fc-VHHb fusions comprising two VHHs that respectively target epitope 1 and 2 potently neutralize SARS-COV-2 WT and the Omicron BA.1 and BA.2 variants. Neutralization assays were performed using VSV particles pseudotyped with either SARS-COV-2 614G spike proteins (A) or spike proteins of the Omicron BA.1 (B) or BA.2 (C) variants. The graphs show the mean GFP fluorescence intensity single dilution series each normalized to mock infected and infected untreated controls that were included in each dilution series.

FIG. 36. Determination of SARS-COV-2 RBD amino acid positions that can lose binding to VHH3.117 and VHH3.89 when mutated, by deep mutational scanning. Deep mutational scanning signal (expressed as % escape) obtained with VHH3.117 (A) or VHH3.89 (B) plotted over the entire length of the SARS-COV-2 RBD (amino acid positions indicated on the ‘site’ axis). (C-D) The amino acid sequence of SARS-COV-2 RBD (spike protein amino acid positions 336-525 of Wuhan-Hu-1 isolate) is shown and the amino acids for which substitutions were associated with loss of binding of VHH3.117 (C) or VHH3.83 (D) as determined by deep mutational scanning are indicated in boxes.

FIG. 37. Binding mode of VHH3.89 and VHH3.117 to the RBD of the SARS-COV-2 (SC2) spike protein. Left, middle and right column show the SC2 RBD (left column), and its complexes with VHH3.89 (middle column) or VHH3.117 (right column), shown in frontal (upper row), and a 90 degree rotated view to the right (middle row) or left (lower row). Complexes of the SARS-COV-2 spike protein in complex with the VHH were determined by cryoEM (see FIG. 38), and are here shown as solvent accessible surface, colored light gray (SC2 RBD), dark gray (VHH3.89) or middle gray (VHH3.117). On the SC2 RBD surface, the residues identified as escape mutations for VHH3.89 and/or VHH3.117 binding as identified by deep mutational scanning (FIG. 36) are shown in stick representation, labelled and highlighted in dark gray; residues proposed by the cryo-EM experiment as forming a minimal common core (or ‘epitope core’; comprising residues R355, N394, Y396, Y464, S514 and E516) for the binding of VHH3.89 and VHH3.117 family member binders are shown in stick representation, colored black, labeled and highlighted by a box. The epitope core forms a continuous surface area encompassing approximately 300 Å2.

FIG. 38. Cryo-EM reconstructions of VHH3.89 and VHH3.117 bound to the SARS-COV-2 spike protein. Electron potential maps of the SARS-COV-2 spike protein (SC2) in complex with VHH3.117 (upper; 3 Å resolution) or VHH3.89 (lower; 3.1 Å resolution), shown in side (left) and top (middle) view. Shown to the right are the refined cryo-EM structures of the SC2-VHH complexes shown in surface representation and with the receptor binding domain and N-terminal domain of the three SC2 protomers labelled RBD1-3 and NTD1-3. In the SC2-VHH3.117 complex the RBD domain in each of the protomers is in conformationally similar up position and bound by a single VHH3.117 each. In the SC2 VHH3.89 complex all three RBD domains are in up position but in different angles relative to the SC2 core. Two VHH3.89 copies are bound, one to the RBD of SC2 protomer 1 (labelled RBD-1), and a second to the RBD of SC2 protomer 2 (RBD-2). RBD-3 is poorly defined in the cryo-EM maps, indicative of a large conformational flexibility. Based on this experiment, VHH3.117 and VHH3.89 are proposed to bind a largely common epitope comprising residues R355, N394, Y396, Y464, S514 and E516, and which are shielded in the RBD down conformation of the apo SC2 protein.

FIG. 39. VHH3.89 and VHH3.117 target a largely overlapping epitope on the SARS-COV-2 spike protein. Structure of the SARS-COV-2 RBD (residues 330-530) shown as solvent accessible surface, and as frontal view relative to the VHH3.89 and VHH3.117 epitopes. On the SC2 RBD surface, the residues identified as escape mutations for VHH3.89 and/or VHH3.117 binding by deep mutational scanning (FIG. 36) are shown in stick representation, labelled and highlighted in dark gray; residues here proposed by the cryo-EM experiment as forming a minimal common core (or ‘epitope core’; comprising residues R355, N394, Y396, Y464, S514 and E516) for the binding of VHH3.89 and VHH3.117 family member binders are shown in stick representation, colored black, labeled and highlighted by a box. The epitope core forms a continuous surface area encompassing approximately 300 Å2. Binding of VHH3.89 to the epitope core of SC2 RBD results in the burying of approximately 290 Å2 surface with a calculated Gibbs free energy of −2.3 kcal/mol (as determined by PDBePISA).

FIG. 40. VHH3.117 and VHH3.89-Fc induce premature shedding of the spike S1 subunit. (A) VHH72-Fc and VHH3.117 induce S1 shedding from cells expressing the SARS-COV-2 spike protein. (B) VHH3.89-Fc induces S1 shedding from cells expressing the SARS-COV-2 spike protein. Anti-S1 Western blot analysis is shown of the growth medium and cell lysates of Raji cells expressing the SARS-COV-2 spike protein (Raji Spike) or not (Raji) incubated for 30 minutes with the indicated VHH constructs or antibodies. The lower an upper triangle at the right side of the blots indicate respectively the S1 spike subunit generated after furin mediated cleavage of the spike protein and cellular uncleaved spike proteins.

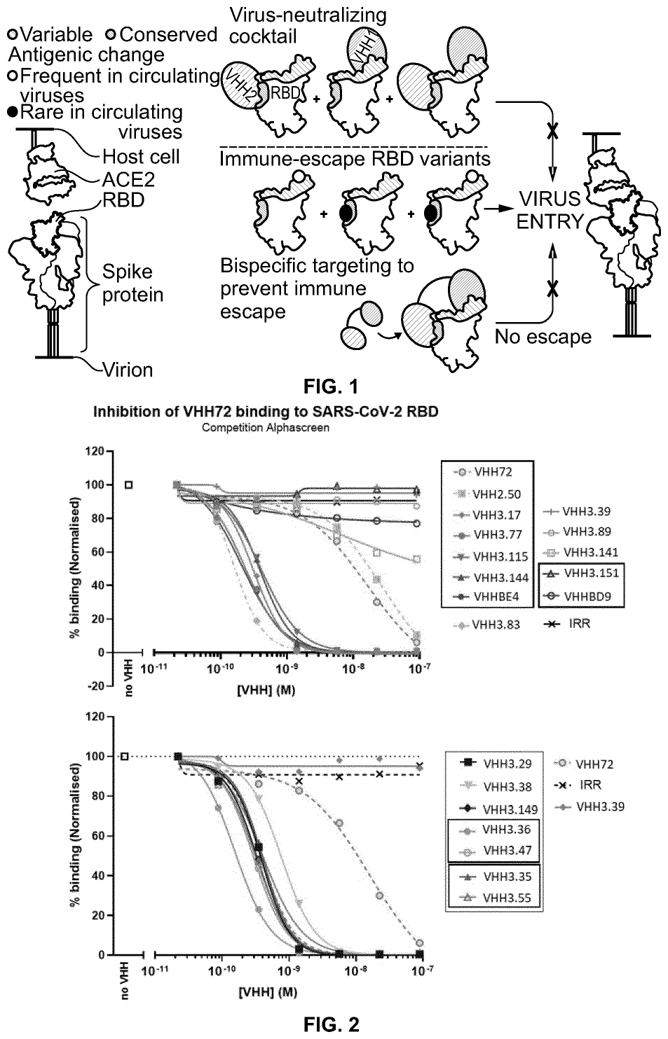

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

‘Nucleotide sequence’, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. With a “chimeric gene” or “chimeric construct” or “chimeric gene construct” is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature. An “expression cassette” comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”.

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions.

By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the a sample or mixture, such as a production host, that are adjacent to said polypeptide. An isolated protein or peptide can be generated by amino acid chemical synthesis or can be generated by recombinant production or by purification from a complex sample. An “isolated nucleic acid” refers to a nucleic acid molecule which has been purified from the molecules which flank it in a naturally-occurring state, or from molecules present in a mixture or complex sample.

The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link. The same applies for the term “inserted in”, wherein one nucleic acid or protein sequence part may be inserted in another sequence by fusing the two sequences genetically, enzymatically or chemically.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant”, “engineered” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “binding pocket” or “binding site” refers to a region of a molecule or molecular complex, that, as a result of its shape and charge, favourably associates with another chemical entity or binding domain, such as a compound, proteins, peptide, antibody or Nb, among others. For antibody-related molecules, the term “epitope” or “conformational epitope” is also used interchangeably herein. The term “pocket” includes, but is not limited to cleft, channel or site. The RBD domain of a Corona virus herein described comprises a binding pocket or binding site which include, but is not limited to a Nanobody binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. A “binding agent”, or “agent” as used interchangeably herein, relates to a molecule that is capable of binding to another molecule, via a binding region or binding domain located on the binding agent, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others.

The RBD domain of a Corona virus herein described comprises a binding pocket or binding site which include, but is not limited to a Nanobody binding site. The term “part of a binding pocket/site” or “partially overlapping epitope” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in receptor binding, or may be residues that are spatially related and define a three-dimensional compartment of the binding pocket, or that confer a conformational function. An “epitope”, as used herein, refers to an antigenic determinant of a polypeptide, constituting a binding site or binding pocket on a target molecule, such as Corona virus RBD domain, more specifically a sarbecovirus RBD domain, even more particularly 2019-nCOV RBD domain. Said epitopes on the RBD domain may be comprise at least one amino acid that is essential for binding the binding agent, though preferably comprise at least 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance, cryo-EM, or other structural analyses. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody” refers to an immunoglobulin (Ig) molecule or a molecule comprising an immunoglobulin (Ig) domain, which specifically binds with an antigen. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies (or VHH antibodies), domain antibodies, and single chain structures, such as a complete light chain or complete heavy chain.

The term “antibody fragment” and “active antibody fragment” or “functional variant” as used herein refer to a protein comprising an immunoglobulin domain or an antigen binding domain capable of specifically binding a RBD present in the Corona virus Spike protein, more specifically the SARS-COV-2 virus spike protein. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. For numbering of the amino acid residues of an IVD different numbering schemes can be applied. For example, numbering can be performed according to the AHo numbering scheme for all heavy (VH) and light chain variable domains (VL) given by Honegger, A. and Plückthun, A. (J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231(1-2), J Immunol Methods. 1999. It should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. Determination of CDR regions may also be done according to different methods, such as the designation based on contact analysis and binding site topography as described in MacCallum et al. (J. Mol. Biol. (1996) 262, 732-745). Or alternatively the annotation of CDRs may be done according to AbM (AbM is Oxford Molecular Ltd.'s antibody modelling package as described on http://www.bioinf.org.uk/abs/index.html), Chothia (Chothia and Lesk, 1987; Mol Biol. 196:901-17), Martin (Abhinandan, and Martin. Molecular Immunology 45 (2008) 3832-3839; as shown in http://bioinf.org.uk/abs/info.html), Kabat (Kabat et al., 1991; 5^{th} edition, NIH publication 91-3242), IMGT (LeFranc, 2014; Frontiers in Immunology. 5 (22): 1-22), and/or alternative annotations including aHo, Gelfand, and Honegger; see, e.g., Dondelinger et al. 2018, Front Immunol 9:2278 for a review). Those annotations exist for numbering amino acids in immunoglobulin protein sequences, though in the present application solely the Kabat numbering is used, or the specific SEQ ID numbering, as indicated. Said annotations further include delineation of CDRs and framework regions (FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target.

VHHs or Nbs are often classified in different sequences families or even superfamilies, as to cluster the clonally related sequences derived from the same progenitor during B cell maturation (Deschaght et al. 2017. Front Immunol. 10; 8: 420). This classification is often based on the CDR sequence of the Nbs, and wherein for instance each Nb family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, having the same effect or functional impact.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat). Humanization typically only concerns substitutions in the FR and not in the CDRs, as this could/would impact binding affinity to the target and/or potency.

The composition or binding agent(s) of the invention comprising binders for binding site 1 and 2, as described herein may appear in a “multivalent” or “multispecific” form and thus be formed by bonding, chemically or by recombinant DNA techniques, together two or more identical or different binding agents. Said multivalent forms may be formed by connecting the building block directly or via a linker, or through fusing the with an Fc domain encoding sequence. Non-limiting examples of multivalent constructs include “bivalent” constructs, “trivalent” constructs, “tetravalent” constructs, and so on. Examples of such bivalent constructs, or homobivalent constructs is herein further described in the appended examples section specifically for ISVD building blocks. The immunoglobulin single variable domains comprised within a multivalent construct may be identical or different, preferably binding to the same or overlapping binding site. In another particular embodiment, the binding agent(s) of the invention are in a “multi-specific” form and are formed by bonding together two or more building blocks or agents, of which at least one binds to binding site 1, as defined herein, and at least one binds to binding site 2, as defined herein, so both forming a binding agent or composition that is capable of specifically binding both epitopes, thus comprising binders with a different specificity. Non-limiting examples of multi-specific constructs include “bi-specific” constructs, “tri-specific” constructs, “tetra-specific” constructs, and so on. To illustrate this further, any multivalent or multi-specific (as defined herein) ISVD of the invention may be suitably directed against two or more different epitopes on the same RBD of Corona virus antigen, or may be directed against two or more different antigens, for example against the Corona RBD and one as a half-life extension against Serum Albumin or Staphylococcal protein A (SpA). Multivalent or multi-specific ISVDs of the invention may also have (or be engineered and/or selected for) increased avidity and/or improved selectivity for the desired Corona RBD interaction, and/or for any other desired property or combination of desired properties that may be obtained by the use of such multivalent or multi-specific immunoglobulin single variable domains. Upon binding the Corona RBD, said multi-specific binding agent or multivalent ISVD may have an additive or synergistic impact on the binding and neutralization of Corona virus, such as SARS-Corona or 2019-novel Corona virus. In another embodiment, the invention provides a polypeptide comprising any of the immunoglobulin single variable domains according to the invention, either in a monovalent, multivalent or multi-specific form. Thus, polypeptides comprising monovalent, multivalent or multi-specific nanobodies are included here as non-limiting examples. The multivalent or multispecific binders or building blocks may be fused directly or fused by a suitable linker, as to allow that the at least two different binding sites can be reached or bound simultaneously by the multispecific agent. Alternatively, at least one ISVD as described herein may be fused at its C-terminus or N-terminus to an Fc domain, for instance an Fc-tail of an Ig, resulting in a corona virus spike protein binding agent of bivalent format wherein two of said VHH-Ig Fcs (or VHH-Fcs), or humanized forms thereof, form a heavy chain only-antibody-type molecule through disulfide bridges in the hinge region of the Fc part. Humanized forms, such as IgG humanized forms, include but are not limited to the IgG humanization variants known in the art, such as C-terminal deletion of Lysine, alteration or truncation in the hinge region, LALA (L234A and L235A) or LALAPG (L234A, L235A, and P329G) mutations as described, among other substitutions in the IgG sequence. In an alternative setup, an Fc fusion is designed by linking the C-terminus of such a bivalent or bispecific binder fused by a linker to an Fc domain, which then upon expression in a host form a multivalent or multispecific-antibody-type molecule through disulfide bridges in the hinge region of the Fc part.

As used herein, a “therapeutically active agent” or “therapeutically active composition” means any molecule or composition of molecules that has or may have a therapeutic effect (i.e. curative or prophylactic effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition of the invention may act as a therapeutically active agent, when beneficial in treating patients infected with corona virus infections, more specifically sarbecovirus infection, such as SARS Corona virus, or patients suffering from SARS-COV-2 infection, or any mutant variants thereof, or more specifically COVID-19. The therapeutically active agent/binding agent or composition may include an agent comprising an ISVD specifically binding the VHH72-epitope as defined herein, and an ISVD specifically binding the VHH3.117-epitope, as defined herein, and, preferably an improved variant binding to the same binding region of both epitopes on the RBD, and more preferably a humanized variant thereof, and may contain or be coupled to additional functional groups, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD or active antibody fragment, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the target RBD of Corona virus and one against a serum protein such as albumin or Surfactant Protein A (SpA)—which is a surface protein abundantly present in the lungs aiding in prolonging half-life)) or into fusions of antibody fragments, in particular immunoglobulin single variable domains, with peptides (for example, a peptide against a serum protein such as albumin). In yet another example, the (variant) ISVD of the invention or bispecific ISVD or multispecific ISVD can be fused to an immunoglobulin Fc domain such as an IgA Fc domain or an IgG Fc domain, such as for example IgG1, IgG2 or IgG4 Fc domains. Examples are further described in the detailed description and shown in the experimental section and are also depicted in the sequence listing.

As used herein, the terms “determining,” “measuring,” “assessing,”, “identifying”, “screening”, and “assaying” are used interchangeably and include both quantitative and qualitative determinations. “Similar” as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring.

A “composition” relates to a combination of one or more active molecules, and may further include buffered solutions and/or solutes such as pH buffering substances, water, saline, physiological salt solutions, glycerol, preservatives, etc. for which a person skilled in the art is aware of the suitability to obtain optimal performance. Suitable conditions as used herein could also refer to suitable binding conditions, for instance when Nbs are aimed to bind a RBD.

A pharmaceutical composition comprising the one or more binding agents or nucleic acid molecule, or recombinant vector as provided herein, optionally comprise a carrier, diluent or excipient. A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. A pharmaceutically effective amount of polypeptides, or conjugates of the invention and a pharmaceutically acceptable carrier is preferably that amount which produces a result or exerts an influence on the particular condition being treated. For therapy, the pharmaceutical composition of the invention can be administered to any patient in accordance with standard techniques. The administration can be by any appropriate mode, including orally, parenterally, topically, nasally, ophthalmically, intrathecally, intracerebroventricularly, sublingually, rectally, vaginally, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN™, PLURONICS™ or PEG and the like. The preparation containing pharmaceutical composition of this invention should be sterilized before injection. This procedure can be done using sterile filtration membranes before or after lyophilization and reconstitution. The pharmaceutical composition is usually filled in a container with sterile access port, such as an i.v. solution bottle with a cork.

### DETAILED DESCRIPTION

The present invention relates to compositions or binding agents comprising binding regions or binding domains, in particular immunoglobulin single variable domains (ISVDs), which specifically interact with at least two different, non-competing epitopes on the Receptor binding domain (RBD) that is part of the spike protein of the sarbecoviruses, such as the SARS-COV-1 Corona virus and the SARS-Cov-2 Corona virus. The interaction between the binding domain(s), in particular the ISVDs, of the agent(s) or of the composition and the spike protein results in a neutralization of the infection capacity of the sarbecoviruses, and wherein the combination of said at least two non-competing binding regions, in particular ISVDs, in one or more agents of the composition results in cross-reactivity and potent prohibition of infection by sarbecoviruses, in the broadest way possible, i.e. in a pan-specific manner. Indeed, by selecting for binders, in particular first and second ISVDs, interacting with the epitopes defined herein as ‘binding site 1’ and ‘binding site 2’, or ‘VHH72-epitope’ and ‘VHH3.117-epitope’, respectively, both being very conserved regions of the RBD among sarbecoviruses, a pan-specific sarbecoviral composition or binding agent can be produced, which allows for reducing the risk to escape mutagenesis.

Moreover, by providing binders specific for said two epitopes ‘binding site 1’ and ‘binding site 2’ of the spike protein, in particular first and second ISVDs, of which the binders for the VHH72-epitope, in particular the first ISVD(s), compete for human receptor (ACE-2 in the case of SARS-COV-1 and -2) binding upon interaction to the RBD, and the binders for the VHH3.117 epitope, in particular the second ISVD(s), neutralize the virus without inhibiting binding of the RBD with the human receptor (ACE-2 in the case of SARS-COV-1 and -2), the resulting bispecific or multispecific binding agent or composition acts via at least two distinct mechanisms in its neutralization. Without wishing to be bound by any theory, binders for the VHH3.117 epitope, in particular the second ISVD(s), may induce S1 shedding and consequently premature spike triggering and may as such not allow the sarbecovirus to complete the infection or entry process into the host cell.

A major advantage of the pan-specific binders described herein is provided by the characteristic of the epitopes both comprising RBD amino acids that are very conserved within the RBD of sarbecoviruses of multiple clades which indicates that the epitopes are stable and not subject of frequent mutational changes. Such sarbecovirus-neutralizing agents are crucial to therapeutic developments for treatment of COVID-19 in view of the multiple emerging SARS-COV-2 variants, some of these being more infectious and/or causing more severe disease symptoms (including in younger people) and/or escaping some of the existing vaccines and/or diagnostic tests. The compositions and binding agents identified herein as well as their applications are described in more detail hereinafter.

SARS-COV-2 contains as structural proteins the spike (S) protein, the envelope (E) protein, the membrane (M) protein, and the nucleocapsid (N) protein. Furthermore, sixteen nonstructural proteins (nsp1-16) have been discerned, and being involved in replication and modifying the host defense. The Nsp12 protein corresponds to a RNA-dependent RNA polymerase (RdRp). Of specific interest in the current invention is the spike or S protein which is a transmembrane glycoprotein forming homotrimers protruding from the viral surface and giving the virus a crown-like look. The spike protein has two subunits: S1 and S2. The S1 subunit comprises an N-terminal domain (NTD), a receptor binding domain (RBD)—as indicated above, the RBD is binding to human ACE-2- and subdomains 1 and 2 (SD1, SD2). The S2 subunit is involved in fusing the membranes of viruses and host cells, and comprises multiple domains: an S2′ protease cleavage site (cleavage by a host protease required for fusion), a fusion peptide (FP), a heptad repeat 1 (HR1) domain, a central helix (CH) domain, a connector domain (CD), a heptad repeat 2 (HR2) domain, a transmembrane (TM) domain, and a cytoplasmic tail (CT) domain (Wang et al. 2020, Front Cell Infect Microbiol 10:587269). In the prefusion conformation, S1 and S2, cleaved at the S1-S2 furin cleavage site during biosynthesis, remain non-covalently bound to each other—this is different from SARS-COV in which S1 and S2 remains uncleaved. In the closed state of the S protein (PDB: 6VXX), the 3 RBD domains in the trimer do not protrude from the trimer whereas in the open state (PDB:6VYB), or “up” conformation, one of the RBD does protrude from the trimer. The S-trimer ectodomain with triangular cross-section has a length of approximately 160-Ångstrom wherein the S1 domain adopts a V-shaped form. Sixteen of the 22 N-linked glycosylation sites per protomer appear glycosylated (Walls et al. 2020, Cell 180:281-292). The RBD domain contains a core beta-sheet region formed by 5 antiparallel strands. Between two of the antiparallel strands is inserted the receptor binding motif (RBM) forming an extended structure (formed by 2 short beta-strands, 2 alpha-helices and loops) containing most of the residues binding to ACE2 (Lan et al. 2020, Nature 581:215-220).

The Sars-Cov-2 Spike protein sequence can be found under/corresponds with or to Genbank Accession: QHQ82464, version QHQ82464.1; and is also defined herein as SARS-COV-2 surface glycoprotein, and as SEQ ID NO:1. Herein, the SARS-COV-2 Spike protein RBD domain region (also defined as Spike receptor binding domain; pfam09408) corresponds with/to amino acids 330-583 of SEQ ID NO:1; or alternatively corresponds with/to amino acids 330-518 of SEQ ID NO:1; or alternatively to amino acids 349-526 of SEQ ID NO:1.

The RBD thus forms the site of interaction with the human receptor: the “Angiotensin converting enzyme 2”, “ACE2”, or “ACE-2” as used herein interchangeably, which refers to the mammalian protein belonging to the family of dipeptidyl carboxydipeptidases, and sometimes classified as EC:3.4.17.23, and herein acts as a receptor for at least human coronaviruses SARS-COV and SARS-COV-2, and NL63/HCoV-NL63 (also known as New Haven coronavirus). UniProtKB identifier of human ACE2 protein: Q9BYF1. Isoform 1 (identifier: Q9BYF1-1) has been chosen as the canonicali sequence. Reference DNA sequence of the human ACE2 gene in GenBank: NC_000023.11. Reference mRNA sequences of human ACE2 in GenBank NM_001371415.1 and NM_021804.3.

In a first aspect of the invention, a composition comprising one or more Corona virus spike protein-specific binding agents is described. A ‘composition’, as used herein, refers to a combination of one or more molecules, present in a formulation that retains the agents activity, specifically the RBD binding and sarbecovirus neutralization activity in this case, thus a functional composition. Said composition may thus be a soluble or solid composition, in addition to said Spike protein binding agent molecule(s) further comprising for instance but not limited to, buffer components, adjuvants, or additional molecules, which may be functional molecules. The composition comprises one or more molecules which constitute one or more binding agents or binding domains which specifically bind the sarbecovirus Spike protein via interaction with its RBD region. More specifically, said composition may thus contain at least two binding agents or at least two binding domains, characterized in that one binding agent or domain specifically binds the RBD region at one binding site, and the second binding agent or domain specially bind the RBD region at another epitope or binding site which is different from the first binding site, and thus resulting in a composition with at least two binding agents or binding domains binding in a non-competing manner to the RBD, possibly simultaneously. Said at least two binding agents or binding domains may be present as one or as several molecule entities in said composition. Preferably, said composition comprises a binding agent that is capable of binding said two non-competing binding sites of the RBD, via two different binding domains present in said binding agent, wherein said binding agent may be a biparatopic or bispecific binding agent, or multivalent or multispecific binding agent. Moreover said composition may still contain additional binding agent(s) or molecules, which optionally bind further binding regions on the same or different epitopes of the spike protein, or other viral proteins, or may even target totally unrelated target proteins. More specifically, said composition comprises one or more Spike protein-specific binding agents or domains wherein said agents or domains are antigen-binding agents of molecules. More specifically, antibody-type molecules, i.e. molecules defined herein as antibodies or immunoglobulin domain-containing molecules specific for the RBD antigenic binding sites described herein. Said antibody composition is envisaged herein to provide for one or more binding agents or binding domains, provided in said composition as one or several molecules, wherein at least one of said molecule(s) is of polypeptidic nature, specifically binding the RBD of the spike protein, as depicted herein in SEQ ID NO:1, through interaction with said RBD at at least 1 of 2 non-competing binding sites. In particular embodiments, said composition provides for at least one or more first immunoglobulin single variable domains (ISVDs) and one or more second ISVDs, wherein said first and second ISVDs specifically bind the RBD of the spike protein, as depicted herein in SEQ ID NO:1, through interaction with said RBD at 2 non-competing binding sites. Said first and second ISVDs may be present in a single molecule or in 2 different molecules. Said binding sites of said one or more binding agents or binding domains of said composition are defined herein as ‘binding site 1’ and ‘binding site 2’, which thus relate to said 2 non-competing, different regions of the RBD that are specifically recognized by the binders disclosed herein. With ‘binders’ is meant herein any molecule with a region that is capable of specifically interacting with the target, more specifically for instance the RBD spike protein target, even more specifically at epitope 1 or ‘binding site 1’ or at epitope 2 or ‘binding site 2’. In the case where the composition contains a single binding agent that is capable to specifically bind both, binding site 1 and binding site 2, as defined herein, said single binding agent is defined as a multispecific agent, or more preferably a biparatopic or bispecific binding agent. In particular embodiments, such multispecific binding agent comprises one or more first ISVD and one or more second ISVD, wherein said first and second ISVDs specifically bind the RBD of the spike protein, as depicted herein in SEQ ID NO:1, through interaction with said RBD at the 2 non-competing binding sites.

Said binding agent may be a polypeptide, or more specifically an antigen-binding domain, or immunoglobulin domain, or an antibody or an antibody fragment, or a single domain antibody or an ISVD, a VHH, or a Nb. Alternatively, said binding agent may also be a small molecule, a conjugate, a chemical, a peptide, a peptidomimetic, an antibodimimetic, or alike.

Components Interacting with Multiple Conserved Regions on the Receptor-Binding Domain (RBD) of the Sarbecovirus Spike Protein as to Reduce Escape Mutant Virus Emergence

As such, the binding agents of the composition of the invention provide for agents (1) capable of neutralizing, inhibiting, blocking or suppressing sarbecoviruses, in particular (2) capable of neutralizing, inhibiting, blocking or suppressing infection with sarbecoviruses or the infective capacity of sarbecoviruses and/or (3) capable of neutralizing, inhibiting, blocking or suppressing replication of sarbecoviruses. For instance, interaction (binding, specific binding) between a binding agent as identified herein and the sarbecovirus spike protein results in a neutralization of the infection capacity or infective capacity of the sarbecovirus, such as determined in any assay as described herein or as known in the art. Another function of the binding agents described herein is that these agents are (4) capable of binding or of specifically binding to a spike protein of sarbecoviruses. In particular, these agents are (5) capable of binding or of specifically binding to the RBD domain or motif, or to part of RBD domain or motif, in a sarbecovirus spike protein, in particular in the spike protein of many different sarbecoviruses, more in particular to a highly conserved epitope in the RBD domain or motif, or to part of RBD domain or motif, in sarbecovirus spike proteins. Independent of their mechanism of action, whether via competition with the human receptor (ACE2 receptor) binding to the RBD or not, for instance through binding to ‘binding site 1’ and ‘binding site 2’, as defined herein, respectively, the binding agents according to the invention are neutralizing sarbecovirus infection efficiently/efficaciously. For instance, and without wishing to be bound by any theory, the binding agents may induce S1 shedding and consequently premature spike triggering and may as such not allow the sarbecovirus to complete the infection or entry process into the host cell.

The composition as described herein comprises at least one ‘binding agent’ or ‘binding domain’, or one or more first ISVDs, specifically binding the spike protein as defined in SEQ ID NO:1 via its binding site located at the RBD, referred to herein as ‘binding site 1’, characterized in that said binding agent or binding domain for binding site 1 or said first ISVD corresponds to the ‘VHH72-epitope’ as described in Wrapp et al. (2020, Cell 184:1004-105) and in PCT/EP2021/052885, and further herein. Moreover said binding agent or binding domain specific for binding site 1, or said first ISVD, is not competing with the (part of) binding agent specifically binding ‘binding site 2’, or the second ISVD, more specifically, said binding agent or binding domain for binding site 1, or said first ISVD, is not competing with the VHH3.117 family members (VHH3.117, 3.42, 3.92, 3.94, 3.180) (as described in the examples and in Saelens et al. EP 21166835.5 and PCT/EP2022/052919), or with the VHH3.89 (as described in PCT/EP2021/052885) and its family members, VHH3_183, and VHH3C_80 (as described in Example 9 herein), more specifically, with any binding agents comprising the CDR1, 2 and 3 regions of any of SEQ ID NOs:22-27 or SEQ ID NOs 85-87, wherein said CDRs are annotated according to any of the annotations described and provided herein. With ‘not competing’ is meant herein as to ‘allow binding’ of said non-competing binders when the binding agent is bound to the binding site 1 of the RBD or vice versa.

Moreover, the structural analysis further demonstrates that said epitope 1, as defined herein, specifically binding the binding agent or binding domain, or the first ISVD, such as for instance VHH72, is occluded in the closed spike conformation that is the dominant one on the native virus. Even in the ‘1-RBD-up’ conformation that can bind the ACE2 receptor, the epitope is positioned such that human monoclonal antibodies cannot easily reach it. Possibly because of this, amidst hundreds of antibodies against other regions of the spike, very few human antibodies thus bind to an epitope that substantially overlaps the VHH72 epitope. Moreover, the epitope is comprised of residues that form crucial packing contacts between the protomers of the trimeric spike. SARS-COV-2 viruses with mutations in this epitope so far remain extremely rare. Consistently, none of the emerging and rapidly spreading viral variant's RBD mutations affect the VHH72 binding site. Antibodies that cross-neutralize SARS-COV-1 and -2 and other viruses of the Sarbecovirus subgenus, as is the case for the binding agents of the present invention, are thus rare and the present binding agents comprising said ISVDs are thereby unique.

CR3022 was reported to be able to bind with purified recombinant 2019-nCOV or SARS-COV-2 RBD (ter Meulen et al. 2006, PLOS Med 3:e237), in a region that partially overlaps with the VHH72 epitope, though, CR3022 does not compete for binding of ACE2 to the SARS-COV-2 RBD, whereas the binding agents of the present invention binding to epitope 1 or alternatively the VHH72-epitope provide for a clear competition with ACE2 for binding with SARS RBD.

In one embodiment, the VHH72-epitope or ‘binding site 1’ of the present invention relates to a well-defined conformational epitope in the RBD of SARS COV making close contact with at least K378, or at least one or more of the residues K378, Y369 and F377, as present on the spike protein of SARS-COV-2, as shown in SEQ ID NO:1. More specifically, said binding site 1 of the Spike protein is defined herein as an epitope comprising at least one or more of the amino acid residues S371, S375, T376, or C379 as set forth in SEQ ID NO: 1, or even more specifically, at least one of L368, Y369, S371, S375, T376, F377, K378, C379 and Y508 as set forth in SEQ ID NO: 1, which is the sequence of the SARS-Cov-2 Spike protein. In particular, said binding agent specific for binding site 1, or said first ISVD, is referred to herein as specifically binding to the SARS-COV-2 Spike protein (SEQ ID NO:1), to at least 3, to at least 4, to at least 5, to at least 6, to at least 7, to at least 8, to at least 9, to at least 10, to at least 11, or to all of the amino acids listed herein, L368, Y369, S371, S375, T376, F377, K378, C379 and Y508 of the SARS-COV-2 spike protein as depicted in SEQ ID NO:1.

Another embodiment relates to a binding agent specifically binding the Corona virus Spike protein, which is defined as a binding agent competing for the epitope 1 as defined herein, or competing with VHH72 binding to the RBD epitope. In certain embodiments, the one or more first ISVDs compete for the epitope 1 as defined herein, or compete with VHH72 binding to the RBD epitope. With ‘competing’ is meant that the binding of VHH72 to the Spike protein as depicted in SEQ ID NO:1 is reduced with at least 30%, or at least 50%, or preferably at least 80% in strength in the presence of said competing binding agent or said first ISVD. More specifically, said competing binding agent or said first ISVD specifically binds an epitope on the Spike protein comprising at least three, at least four, at least five, at least 6 or more of the residues L368, Y369, S371, S375, T376, F377, K378, C379 and Y508 of the Spike protein of SARS-Cov-2, as depicted in SEQ ID NO:1, so as to provide an overlapping epitope, more specifically at least binding to 2 of its residues, or at least to 3, or at least 4, or at least 6 of its residues. In a specific embodiment the competing binding agent or the first ISVD specifically binds to residues K378, Y369 and F377. In another specific embodiment the competing binding agent or the first ISVD specifically binds to residues K378, Y369 and F377 as depicted in SEQ ID NO:1, and said competing binding agent or said first ISVD competes for ACE2 receptor binding to the Spike protein and/or RBD domain. Specifically, said competing binding agent or said first ISVD specifically binds an epitope on the Spike protein comprising at least a part of the residues L368, Y369, S371, S375, T376, F377, K378, C379 and Y508 of the Spike protein of SARS-Cov-2, as depicted in SEQ ID NO:1, so as to provide an overlapping epitope, more specifically at least binding 30% of the residues, or at least 50% of the residues, or at least 80% of the residues, and/or specifically including residues K378, and/or F377.

The composition as described herein comprises at least one binding agent or binding domain, or one or more second ISVDs, specifically binding the spike protein as defined in SEQ ID NO: 1 via its binding site located at the RBD, referred to herein as ‘binding site 2’, characterized in that said binding agent for binding site 2 or said second ISVD corresponds to the ‘VHH3.117-epitope’ as described in EP 21166835.5 and PCT/EP2022/052919 and further herein. Moreover, said binding agent or binding domain specific for binding site 2 or said second ISVD is not competing with the (part of the) binding agent specifically binding site 1, or the first ISVD, as defined herein, more specifically, not competing with the known immunoglobulin VHH72 (Wrapp et al. 2020, Cell 184:1004-105), and/or also not competing with the known immunoglobulin CR3022 (ter Meulen et al. 2006, PLOS Med 3:e237; Tian et al. 2020, Emerging Microbes & Infections 9:382-385), and/or are not competing with the known immunoglobulin CB6 (Shi et al. 2020, Nature 584:120-124), and/or not competing with the known immunoglobulin S309 (Pinto et al. 2020, Nature 583:290-295), all for binding or for specifically binding to the spike protein (or RBD domain therein) of sarbecoviruses.

With ‘not competing’ is meant her as to ‘allow binding’ of said non-competing binders when the binding agent is bound to the binding site 2 of the RBD, or vice versa. Thus the binding agent specifically binding ‘epitope 2’ or ‘binding site 2’ of the RBD, as used interchangeably herein, or the second ISVD, is characterized by a different spike protein/RBD binding pattern compared to the spike protein/RBD binding pattern of the (part of the) binding agent specifically binding site 1, or the first ISVD, and/or any of the immunoglobulins CR3022, VHH72, CB6, or S309. Alternatively, these binding agents specific for binding site 2 of the RBD, or these second ISVDs, allow binding of CR3022, VHH72, CB6 or S309 to the sarbecovirus RBD or spike protein when these binding agents or second ISVDs are themselves bound to the sarbecovirus RBD. Alternatively, the binding agent or the second ISVD itself can bind to a sarbecovirus RBD to which CR3022, VHH72, CB6 or S309 is bound.

In one particular embodiment, the binding agent, or part of the binding agent, that specifically interacts with binding site 2 of the RBD, as defined herein, or the second ISVD, may be defined/may be characterized in that the agent or the second ISVD is binding to the sarbecovirus spike protein Receptor Binding Domain (SPRBD), is allowing binding of Angiotensin-Converting Enzyme 2 (ACE2) to SPRBD when the sarbecovirus binding agent or the second ISVD itself is bound to SPRBD, is at least neutralizing SARS-COV-2 and SARS-COV-1, and is binding to at least one of the amino acids Thr393 (or alternatively Ser393 in some sarbecoviruses), Asn394 (or alternatively Ser394 in some sarbecoviruses), Val395, or Tyr396 of the SARS-COV-2 spike protein as defined in SEQ ID NO:1.

Said binding to said binding site 2 wherein at least one of these residues is bound further characterize this binding site 2 as an epitope in the spike protein or RBD that is different from the epitope as bound by immunoglobulin mAb52 or Fab52 (Rujas et al. 2020, Biorxiv 2020.10.15.341636v1); and/or the epitope as bound by immunoglobulin nb34 (Xiang et al. 2020, Science 370:1479-1484); and/or the epitope as bound by immunoglobulin nb95 (Xiang et al. 2020, Science 370:1479-1484); and/or the epitope as bound by immunoglobulins n3088 and/or n3130 (Wu et al. 2020, Cell Host Microbe 27:891-898); and/or the epitope as bound by immunoglobulins n3086 and/or n3113 (Wu et al. 2020, Cell Host Microbe 27:891-898).

More specifically, said binding agent specific for binding site 2 of the spike protein, or said second ISVD, is referred to herein as specifically binding to the SARS-COV-2 Spike protein (corresponding to the amino acid sequence of SEQ ID NO:1), to at least one of the amino acids Thr393 (or alternatively Ser393 in some sarbecoviruses), Asn394 (or alternatively Ser394 in some sarbecoviruses), Val395, or Tyr396; and/or in particular, to at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or in particular, to at least one of the amino acids Ser 514, Glu516, or Leu518; and/or in particular, to amino acid Arg357 (or alternatively Lys357 in some sarbecoviruses). In particular, said binding agent specific for binding site 2, or said second ISVD, is referred to herein as specifically binding to the SARS-COV-2 Spike protein (SEQ ID NO:1), to at least 3, to at least 4, to at least 5, to at least 6, to at least 7, to at least 8, to at least 9, to at least 10, to at least 11, or to all of the amino acids listed herein, R357, T393, N394, V395, Y396, K462, F464, E465, R466, S514, E516, and L518 of the SARS-COV-2 spike protein as depicted in SEQ ID NO:1.

Optionally, these agents, or second ISVD(s) are binding or specifically binding to a sarbecovirus spike protein wherein Cys336 (conserved between sarbecovirus clades) is forming an intramolecular disulfide bridge and/or are binding or specifically binding to a sarbecovirus Spike protein wherein Cys391 (conserved between sarbecovirus clades) is forming an intramolecular disulfide bridge; in particular, Cys336 may be forming an intramolecular disulfide bridge with Cys361 (conserved between sarbecovirus clades) and/or Cys391 may be forming an intramolecular disulfide bridge with Cys525 (conserved between sarbecovirus clades). Optionally, these binding agents or binding domains, or second ISVD(s), are binding or specifically binding to a sarbecovirus Spike protein wherein amino acid 365 is a tyrosine (Tyr365; conserved between sarbecovirus clades) and/or are binding or specifically binding to a sarbecovirus Spike protein wherein amino acid 392 is a phenylalanine (Phe392; conserved between sarbecovirus clades) and/or are binding or specifically binding to a sarbecovirus Spike protein wherein amino acid 393 is a threonine (Thr393; or alternatively Ser393 in some sarbecoviruses), and/or are binding or specifically binding to a sarbecovirus Spike protein wherein amino acid 395 is a valine (Val395; or alternatively Ser393 in some sarbecoviruses) and/or are binding or specifically binding to a sarbecovirus Spike protein wherein amino acid 518 is a leucine (Leu518).

In certain embodiments, the binding agent or binding domain specific for binding site 2 of the spike protein, or the second ISVD, binds or specifically binds to at least one of the amino acids Asn394 (or alternatively Ser394 in some sarbecoviruses), or Tyr396; and/or in particular, this binding agent or binding domain, or this second ISVD, binds or specifically binds to Phe464 (or alternatively Tyr464 in some sarbecoviruses); and/or in particular, this binding agent or binding domain, or this second ISVD, binds or specifically binds to at least one of the amino acids Ser514 or Glu516; and/or in particular, this binding agent or binding domain, or this second ISVD, binds or specifically binds to Arg355. In certain embodiments, the binding agent or binding domain specific for binding site 2 of the spike protein, or the second ISVD, binds or specifically binds to at least one of the amino acids Asn394 (or alternatively Ser394 in some sarbecoviruses), Tyr396, Phe464, Ser514, Glu516, and Arg355. In certain embodiments, the binding agent or binding domain specific for binding site 2 of the spike protein, or the second ISVD, binds or specifically binds to at least two of the amino acids Asn394 (or alternatively Ser394 in some sarbecoviruses), Tyr396, Phe464, Ser514, Glu516, and Arg355. In certain embodiments, the binding agent or binding domain specific for binding site 2 of the spike protein, or the second ISVD, binds or specifically binds to at least three of the amino acids Asn394 (or alternatively Ser394 in some sarbecoviruses), Tyr396, Phe464, Ser514, Glu516, and Arg355. In certain embodiments, the binding agent or binding domain specific for binding site 2 of the spike protein, or the second ISVD, binds or specifically binds to at least four of the amino acids Asn394 (or alternatively Ser394 in some sarbecoviruses), Tyr396, Phe464, Ser514, Glu516, and Arg355. In certain embodiments the binding agent or binding domain specific for binding site 2 of the spike protein, or the second ISVD, binds or specifically binds to at least five of the amino acids Asn394 (or alternatively Ser394 in some sarbecoviruses), Tyr396, Phe464, Ser514, Glu516, and Arg355. In certain embodiments, the binding agent or binding domain specific for binding site 2 of the spike protein, or the second ISVD, binds or specifically binds to all six of the amino acids Asn394 (or alternatively Ser394 in some sarbecoviruses), Tyr396, Phe464, Ser514, Glu516, and Arg355.

In certain embodiments, the binding agent or binding domain specific for binding site 2 of the spike protein, or the second ISVD, binds or specifically binds such that parts of the agent, the domain or the ISVD come within 4 Angstrom of at least Tyr396, Ser514, and Glu516. In certain embodiments, the binding agent or binding domain specific for binding site 2 of the spike protein, or the second ISVD, binds or specifically binds to at least Tyr396, Ser514, and Glu516. In certain embodiments, the binding agent or binding domain specific for binding site 2 of the spike protein, or the second ISVD, binds or specifically binds such that parts of the agent, domain or ISVD come within 4 Angstrom of at least Asn394 (or alternatively Ser394 in some sarbecoviruses), Tyr396, Ser514, and Glu516. In certain embodiments, the binding agent or binding domain specific for binding site 2 of the spike protein, or the second ISVD, binds or specifically binds to at least Asn394 (or alternatively Ser394 in some sarbecoviruses), Tyr396, Ser514, and Glu516. In certain embodiments, the binding agent or binding domain specific for binding site 2 of the spike protein, or the second ISVD, binds or specifically binds such that parts of the agent, domain or ISVD come within 4 Angstrom of at least Asn394 (or alternatively Ser394 in some sarbecoviruses), Tyr396, Phe464, Ser514, and Glu516. In certain embodiments, the binding agent or binding domain specific for binding site 2 of the spike protein, or the second ISVD, binds or specifically binds to at least Asn394 (or alternatively Ser394 in some sarbecoviruses), Tyr396, Phe464, Ser514, and Glu516.

Optionally, the binding agent or binding domain specific for binding site 2 of the spike protein, or the second ISVD, further binds or specifically binds to amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518, such as further binds or specifically binds to at least two, or in increasing order of preference at least three or all four of amino acid Arg357 (or alternatively Lys357 in some sarbecoviruses) and/or Lys462 (or alternatively Arg462 in some sarbecoviruses) and/or Glu465 (or alternatively Gly465 in some sarbecoviruses) and/or Arg466 and/or Leu518.

In particular, the binding agent or binding domain specific for binding site 2 of the spike protein, or the second ISVD, may bind or specifically bind whereby a binding interface is generated (for example, as determined by PDBePISA) that covers at least 25%, at least 33%, at least 50%, or at least 75% of the RBD surface area circumferentially defined by R355, N394, Y396, F464, S514 and E516. The RBD surface area that is contacted can be calculated to optionally include the intervening surface area that is sterically between these residues.

The amino acids and amino acid numbering referred to hereinabove as present on the Spike protein sequence is relative to/corresponding to the SARS-COV-2 Spike protein as defined in SEQ ID NO:1; corresponding amino acids in spike proteins or RBD domains of other sarbecoviruses can be easily determined by aligning multiple amino acid sequences.

In certain embodiments, the binding agent, or part of the binding agent, that specifically interacts with binding site 2 of the RBD, as defined herein, or the second ISVD, may be defined/may be characterized in that the binding agent or the second ISVD induces S1 shedding.

Another embodiment relates to a binding agent specifically binding the Corona virus Spike protein, which is defined as a binding agent competing for the epitope 2 as defined herein, or competing with VHH3.117 binding to the RBD epitope. In certain embodiments, the one or more second ISVDs compete for the epitope 2, or compete with VHH3.117 binding to the RBD epitope. With ‘competing’ is meant that the binding of VHH3.117 to the Spike protein as depicted in SEQ ID NO:1 is reduced with at least 30%, or at least 50%, or preferably at least 80% in strength in the presence of said competing binding agent or said second ISVD. More specifically, said competing binding agent or said second ISVD specifically binds an epitope on the Spike protein comprising at least three, at least four, at least five, at least 6 or more of the residues R357, T393, N394, V395, Y396, K462, F464, E465, R466, S514, E516, and L518 of the Spike protein of SARS-Cov-2, as depicted in SEQ ID NO:1, so as to provide an overlapping epitope, more specifically at least binding to 2 of its residues, or at least to 3, or at least 4, or at least 6 of its residues. In a specific embodiment the competing binding agent or second ISVD specifically binds to residues T393, N394, V395, or Y396 of SEQ ID NO:1. In another specific embodiment the competing binding agent or second ISVD specifically binds to residues T393, N394, V395, or Y396 as depicted in SEQ ID NO:1, and said competing binding agent or said second ISVD does not compete for ACE2 receptor binding to the Spike protein and/or RBD domain. Specifically, said competing binding agent or second ISVD specifically binds an epitope on the Spike protein comprising at least a part of the residues R357, T393, N394, V395, Y396, K462, F464, E465, R466, S514, E516, and L518 of the Spike protein of SARS-Cov-2, as depicted in SEQ ID NO:1, so as to provide an overlapping epitope, more specifically at least binding 30% of the residues, or at least 50% of the residues, or at least 80% of the residues, and/or specifically including residues T393, N394, V395, Y396 of SEQ ID NO:1.

As referred herein, the ‘not competing with the human receptor, more specifically ACE2 for SARS-COV-1 and -2, for binding a sarbecovirus RBD’ means allowing binding of the receptor and the sarbecovirus RBD when the binding agents or the (second) ISVDs, are themselves bound to the sarbecovirus RBD (alternatively, the binding agent or the (second) ISVD itself can bind to a sarbecovirus RBD to which the receptor is bound); vice versa ‘competing with the receptor for binding the RBD’ means that binding of the human receptor to the RBD is not occurring (or partially occurring, or slower occurring as compared to a control) when the binding agents or the (first) ISVDs, is bound to the RBD (alternatively, the binding agent, or the (first) ISVD itself cannot bind to a sarbecovirus RBD to which the receptor is bound). The binding agents or binding domains, as used interchangeably herein, are thus capable of neutralizing sarbecovirus, specifically SARS-COV virus infection, through binding via at least two different binding sites, the binding site 1 and binding site 2 on the RBD, as defined herein, which results in a complex formed by the binding agent(s) or domain(s) bound to the RBD, which is capable of outcompeting or blocking ACE2 by having the binding agent interacting with the RBD via binding site 1, or through the one or more first ISVDs, and in addition by triggering a modus operandi for neutralization of the virus that is different from blocking ACE2 binding to the RBD, via its binding to binding site 2, as defined herein, or through the one or more second ISVDs. Such a binding agent or binding composition specific for the Spike protein of sarbecoviruses, interacting at two conserved, rarely mutating sites of the RBD region, has not been described elsewhere, and is in our opinion a very promising route to the development of pan-specific corona virus agents.

A further functional characteristic of the composition comprising said one or more binding agents or binding domains for binding to epitope 1 and epitope 2 of the RBD as described herein, in particular one or more binding agents comprising one or more first ISVDs and one or more second ISVDs, is that two or more conserved epitopes in the spike protein or RBD of many sarbecoviruses can be specifically bound by said composition. In particular, the epitopes or binding site 1 and binding site 2 are conserved between different clades of sarbecoviruses. With ‘conserved’ is meant herein that the amino acid residues constituting said epitope are the same or very similar in nature or side chain composition, so that the interaction of said binding agent or binding domain, in particular said ISVDs, with said conserved epitope is retained even when conserved substitutions are present as compared to the amino acid residues disclosed herein for SEQ ID NO:1, and the effect on efficacy and potency is similar or in the same order of magnitude. In particular, the binding site 1 epitope has been described in Schepens et al. (PCT/EP2021/052885) and is conserved between clade 1a, 1b, (to some extent the RBDs of) clade 2 and/or 3 of bat SARS-related sarbecoviruses, and the binding site 2 epitope has been described in Saelens et al. (EP21166835.5 and PCT/EP2022/052919) and is conserved between clade 1.A, clade1.B, clade 2, and clade 3 sarbecoviruses.

A further functional characteristic of the composition comprising said one or more binding agents or binding domains described herein is that these agents or domains specifically binding site 1 and/or binding site 2, or said first and second ISVDs, neutralize SARS-COV-2 and SARS-COV-1, as for instance shown in a pseudotype virus neutralization assay, and/or preferably as shown in live virus assays.

The functional characteristics of the composition and/or binding agents according to the invention can in general be determined by methodology as e.g. employed in the Examples described herein, or as described in some of the hereinabove cited and other publications, or in the patent applications disclosing each of the single binding sites as referred to herein (PCT/EP2021/052885, and EP21166835.5 or PCT/EP2022/052919). Determination of the sarbecovirus spike protein epitope or sarbecovirus RBD domain epitope can be performed by means of e.g. binding competition experiments (such as outlined in the Examples herein or in many of the hereinabove cited publications), or e.g. by mutational analysis (such as outlined in the Examples herein), or e.g. by any means of determining interaction at the 3D-level, including in silico modeling, X-ray crystallography, Cryo-EM, NMR, spin-labeling, or Hydrogen Deuterium Exchange (HDX)-MS.

Interaction of a binding agent as described herein to a sarbecovirus spike protein or RBD domain therein can be derived from structural models. In particular, the agent's interaction with binding site 1 or binding site 2 of the RBD, as defined herein, can be described in terms of intermolecular distances between an atom of the binding agent (e.g. an amino acid or an amino acid side chain or an amino acid hydrogen) and an atom of the sarbecovirus spike protein or RBD domain therein (e.g. an amino acid or an amino acid side chain or an amino acid hydrogen). Algorithms exist by which binding free energy of complexes are estimated, such as FastContact (Champ et al. 2007, Nucleic Acids Res 35:W556-W560). In the FastContact algorithm, the range of desolvation interaction can be adapted, e.g. 6 Ångström (potential going down to zero between 5 and 7 Ångström) or 9 Ångström (potential going down to zero between 8 and 10 Ångström); electrostatic and van der Waals energy are other components used by the FastContact algorithm.

Thus, interaction of a binding agent as described herein to a sarbecovirus spike protein or RBD domain therein can be derived from structural models by defining an interaction between an atom of the binding partner and an atom of the sarbecovirus spike protein or RBD domain therein (as described hereinabove) as a binding if the distance between the two atoms is e.g. between 1 Ångström (Å) and 10 Å, between 1 Å and 9 Å, between 1 Å and 8 Å, between 1 Å and 7 Å, between 1 Å and 6 Å, between 1 Å and 5 Å, between 1 Å and 4 Å, between 1 Å and 3 Å, between 1 Å and 2 Å, and depending on the resolution at which the structure has been resolved, defining the lower limit. Alternatively, residues of the sarbecovirus spike protein or RBD domain therein are in ‘in contact’ with residues of the binding agent or partner, and such ‘contact’ can be defined herein as (intermolecular) contacts between residues with a distance of 4 Å or less, of 5 Å or less, of 6 Å or less, of 7 Å or less, of 8 Å or less, of 9 Å or less, or of 10 Å or less.

In a specific embodiment, said composition comprising one or more binding agents or binding domains specifically binding the VHH72-epitope (or epitope 1 or binding site 1 as used interchangeably herein), and the VHH3.117-epitope (or epitope 2 or binding site 2 as used interchangeably herein), is characterized in that at least one of said binding agents or binding domains comprises an immunoglobulin single variable domain (ISVD), more preferably, in that at least for each binding site 1 and 2 said binding agent or domain comprises an ISVD, in particular one or more first ISVD and one or more second ISVD, respectively, wherein said ISVDs, in particular said first and second ISVDs, are capable of interacting with epitope 1 and 2, resp.

In particular, the binding agent or binding domain is or comprises one or more complementary determining regions (CDRs) of an immunoglobulin single variable domain (ISVD) as described herein, or comprises one or more ISVDs as described herein, and binds to a part of the sarbecovirus spike protein or RBD domain as described in detail hereinabove (the epitope 1 or epitope 2 on the RBD).

In particular, parts of the binding agents or binding domains (such as amino acids (or parts thereof) of the herein described CDRs and/or ISVDs), are contacting or interacting with a distance of between 1 Ångström (Å) and 10 Å, between 1 Å and 9 Å, between 1 Å and 8 Å, between 1 Å and 7 Å, between 1 Å and 6 Å, between 1 Å and 5 Å, between 1 Å and 4 Å, between 1 Å and 3 Å, between 1 Å and 2 Å; or of 4 Å or less, 5 Å or less, 6 Å or less, 7 Å or less, 8 Å or less, 9 Å or less, or 10 Å or less: with at least one of the amino acids of the VHH72- or VHH3.117-epitope as described herein. More specifically said binding agent specific for binding site 2, or said second ISVD, is in contact or interacting with at least one of Y369, F377, K378, L368, S371, S375, T376, C379, and/or Y508 of the RBD on the Spike protein as presented in SEQ ID NO:1.

More specifically said binding agent specific for binding site 2, or said second ISVD, is in contact or interacting with at least one of Thr393 (or alternatively Ser393 in some sarbecoviruses), Asn394 (or alternatively Ser394 in some sarbecoviruses), Val395, or Tyr396; and/or with at least one of the amino acids Lys462 (or alternatively Arg462 in some sarbecoviruses), Phe464 (or alternatively Tyr464 in some sarbecoviruses), Glu465 (or alternatively Gly465 in some sarbecoviruses) or Arg466; and/or with at least one of the amino acids Ser514, Glu516, or Leu518; and/or with amino acid Arg357 (or alternatively Lys357 in some sarbecoviruses) of the RBD on the Spike protein as presented in SEQ ID NO:1. As mentioned, corresponding amino acids in spike proteins or RBD domains of other sarbecoviruses can be easily determined by aligning multiple amino acid sequences.

The binding agents or binding domains of the composition according to the current invention are in another aspect structurally defined as polypeptidic binding agents (i.e. binding agents comprising a peptidic, polypeptidic or protein moiety, or binding agents comprising a peptide, polypeptide, protein or protein domain) or polypeptide binding agents (i.e. binding agents being peptides, polypeptides or proteins). More in particular, the binding agents according to the current invention can be structurally defined as polypeptidic or polypeptide binding agents or domains comprising a complementarity determining region (CDR) as comprised in any of the immunoglobulin single variable domains (ISVDs) defined hereinafter. More in particular, the binding agents or domains of the composition according to the current invention can in one embodiment be structurally defined as polypeptidic or polypeptide binding agents comprising at least CDR3 as comprised in an immunoglobulin single variable domains (ISVDs) as defined hereinafter. In another embodiment, the binding agents according to the current invention can be structurally defined as polypeptidic or polypeptide binding agents comprising at least two of CDR1, CDR2 and CDR3 (e.g. CDR1 and CDR3, CDR2 and CDR3, CDR1 and CDR2), or all three of CDR1, CDR2 and CDR3, as comprised in an immunoglobulin single variable domains (ISVDs) as defined hereinafter.

As outlined and defined herein (see definitions), many systems or methods (Kabat, Martin, MacCallum, IMGT, AbM, aHo, Chothia, Gelfand, Honegger; see, e.g., Dondelinger et al. 2018, Front Immunol 9:2278 for a review) exist for numbering amino acids in immunoglobulin protein sequences, including for delineation of CDRs and framework regions (FRs) in these protein sequences. These systems or methods are known to a skilled artisan who thus can apply these systems or methods on any immunoglobulin protein sequences without undue burden. A binding agent or binding domain of the composition specifically binding to binding site 1, or a first ISVD, as described herein may thus e.g. be characterized in that it is comprising the complementarity determining regions (CDRs) present in any of SEQ ID NOs: 2-21, 90 or SEQ ID NOs:95-98, wherein the CDRs are annotated according to Kabat, Martin, MacCallum, IMGT, AbM, aHo, Chothia, Gelfand, or Honegger.

More in particular such CDRs, as for instance but not limited to Kabat annotation are comprised in any of VHH72-epitope binding ISVDs as listed in Tables 5 disclosed herein, more specifically, the said binding agents or domains comprising an ISVD specifically binding site 1 or a first ISVD comprise in specific embodiments the following CDR1, CDR2, and CDR3 sequence selected from:
CDR1 sequences provided in SEQ ID NOs:28-37, or 141-143
CDR2 sequences provided in SEQ ID NO:38-50, 144 or 145
CDR3 sequences provided in SEQ ID NO:51-61, or 146.

A particular binding agent capable of binding or specifically binding to ‘binding site 1’ as defined herein, comprises an ISVD comprising a CDR1, a CDR2 and a CDR3 as present in SEQ ID NO:90, wherein the CDR1, CDR2 and CDR3 are annotated according to any one of Kabat, MacCallum, IMGT, AbM, Martin or Chothia, is also disclosed herein. An aspect of the present specification thus also discloses this binding agent as such. In particular, said binding agent may comprise a CDR1 defined by/set forth in SEQ ID NO:33, a CDR2 defined by/set forth in SEQ ID NO:144, and a CDR3 defined by/set forth in SEQ ID NO:54. Said binding agent may further be characterized in that it comprises at least one, or a particular combination of two, three or all of the framework regions (FRs) as present in SEQ ID NO:90, wherein the FR or FRs are annotated according to any one of Kabat, MacCallum, IMGT, AbM, Martin or Chothia, or any variant of said FR or FRs, wherein said variant FR or FRs each independently are at least 80%, 85%, 90% or 95% identical to, or have at most 10, such as 1, 2, 3, 4, 5, 6, 7, 8, 9 or 10, amino acid substitutions, deletions or additions, such as preferably conservative and/or humanizing substitutions, compared to the FR or FRs present in SEQ ID NO:90, wherein the FR or FRs are annotated according to any one of Kabat, MacCallum, IMGT, AbM, Martin or Chothia. In particular, said binding agent may comprise (an ISVD comprising) an amino acid sequence with at least 80% or 85%, preferably 90% or 95%, identity to the amino acid sequence defined by/set forth in SEQ ID NO:90, wherein the non-identity or variability is preferably in FR amino acid residues. In particular, such non-identity or variability may be introduced to obtain a humanized variant of an ISVD defined by/set forth in SEQ ID NO:90. More particularly, said binding agent may comprise or consist of an ISVD defined by/set forth in SEQ ID NO:90.

Said binding agent may be functionally characterized as being capable of binding or specifically binding a Sarbecovirus spike protein, in particular said binding agent may be capable of binding or specifically binding the RBD of the Sarbecovirus spike protein, or a part thereof. In particular, said binding agent may bind or specifically bind to the same epitope as VHH72 or a binding agent comprising the CRD1, CDR2 and CDR3 as present in any one of SEQ ID NOs: 2-21 or 95-98, wherein the CDR1, CDR2 and CDR3 are annotated according to any one of Kabat, MacCallum, IMGT, AbM, Martin or Chothia and/or compete with VHH72 or a binding agent comprising the CRD1, CDR2 and CDR3 as present in any one of SEQ ID NOs: 2-21 or 95-98, wherein the CDR1, CDR2 and CDR3 are annotated according to any one of Kabat, MacCallum, IMGT, AbM, Martin or Chothia, for binding to the Sarbecovirus spike protein, RBD or part thereof. More particularly, said binding agent may bind at least one, preferably two or all, of the amino acid residues Y369, F377, and K378 of the SARS-COV-2 spike protein as depicted in SEQ ID NO:1, and optionally one or more of the amino acid residues L368, S371, S375, T376, C379 and Y508 of the SARS-COV-2 spike protein as depicted in SEQ ID NO:1.

Said binding agent may also be characterized in that it competes with ACE2 receptor for binding a Sarbecovirus spike protein or RBD. Said binding agent may further be characterized as being capable of neutralizing a Sarbecovirus, in particular as being capable of neutralizing at least SARS-COV-2 and SARS-CoV-1.

This binding agent may be in a monovalent form or in multivalent form. In the multivalent form, two or more of the ISVDs may be fused directly or via a linker, or the ISVD may be fused to an Fc domain. Further disclosed herein is a (isolated) nucleic acid molecule comprising a polynucleotide sequence encoding this binding agent, a vector comprising this nucleic acid molecule and a cell comprising this nucleic acid molecule or this vector, or a cell expressing this binding agent.

Also disclosed herein is a pharmaceutical composition comprising this binding agent, or this nucleic acid molecule or vector as described above, and a pharmaceutically acceptable carrier; as well as a kit such as a diagnostic kit comprising this binding agent.

Further disclosed herein is this binding agent, or this nucleic acid molecule or vector as described above, or this pharmaceutical composition or this kit as described above for use in medicine, in particular for use in the prevention or treatment of a Sarbecovirus infection such as a SARS-COV-2 or SARS-COV-1 infection such as for use in passive immunisation of a subject.

A binding agent or binding domain of the composition specifically binding to binding site 2, or a second ISVD, as described herein may thus e.g. be characterized in that it is comprising the complementarity determining regions (CDRs) present in any of the VHH3.117 family VHHs, as present in SEQ ID NOs: 22-27 or any of the VHH3.89 family VHHs, as present in SEQ ID NOs: 85-87, wherein the CDRs are annotated according to Kabat, Martin, MacCallum, IMGT, AbM, aHo, Chothia, Gelfand, or Honegger.

More in particular such CDRs, as for instance but not limited to Kabat annotation are comprised in any of VHH3.117-epitope binding ISVDs as listed in Table 6 disclosed herein, more specifically, the said binding agents or domains comprising an ISVD specifically binding site 2 or a second ISVD comprise in specific embodiments the following CDR1, CDR2, and CDR3 sequence selected from:
CDR1 sequences provided in SEQ ID NOs:62-63, or 131-132
CDR2 sequences provided in SEQ ID NO:64-67, or 133-134
CDR3 sequences provided in SEQ ID NO:68-69, or 135-137.

Alternatively, said binding agent or binding domain specifically binding site 2 is or comprises an ISVD, in particular a second ISVD, comprising the (consensus) CDR1, CDR2, and CDR3 of the VHH3.117 family, as depicted in SEQ ID NO:70, 71 and 72, resp. or any humanized variant thereof, in particular the humanized variant thereof wherein the CDR3 sequence contains 2 substitutions as compared to SEQ ID NO:72, more specifically amino acid substitutions of the 2 Methionine residues.

Said consensus CDRs for the VHH3.117 family being more specifically defined as CDR1: IXDMGW, wherein X (Xaa) at position 2 is S (Ser, serine) or N (Asn, asparagine)(SEQ ID NO:70). More in particular:
CDR1 can be defined as ISDMGW (SEQ ID NO:62; comprised in VHH3.117, VHH3.92, VHH3.94 and VHH3.180) or INDMGW (SEQ ID NO:63; comprised in VHH3.42);
CDR2: TITKXGXTNYAXSXXG, wherein X (Xaa) at position 5 is T (Thr, threonine) or S (Ser, serine), X (Xaa) at position 7 is S (Ser, serine) or N (Asn, asparagine), X (Xaa) at position 12 is D (Asp, aspartic acid) or N (Asn, asparagine), X (Xaa) at position 14 is A (Ala, alanine) or V (Val, valine), and X (Xaa) at position 15 is Q (Gln, glutamine) or K (Lys, lysine) (SEQ ID NO:71). More in particular, CDR2 can be defined as TITKTGSTNYADSAQG (SEQ ID NO:64; comprised in VHH3.117 and VHH3.180), TITKTGNTNYADSAQG (SEQ ID NO:65 comprised in VHH3.92), TITKSGSTNYANSAQG (SEQ ID NO:66; comprised in VHH3.94), or TITKTGSTNYADSVKG (SEQ ID NO:67; comprised in VHH3.42);
CDR3: WLXYGMGPDYYGME, wherein X (Xaa) at position 3 is P (Pro, proline) or L (Leu, leucine) (SEQ ID NO: 72). More in particular, CDR3 can be defined as WLPYGMGPDYYGME (SEQ ID NO:68; comprised in VHH3.117, VHH3.92, VHH3.94 and VHH3.42), or WLLYGMGPDYYGME (SEQ ID NO:69; comprised in VHH3.180).

Said binding agent or binding domain specifically binding site 2 may also be or comprise an ISVD, in particular a second ISVD, comprising the (consensus) CDR1, CDR2, and CDR3 of the VHH3.89 family, as depicted in SEQ ID NO:138, 139 and 140, resp. or any humanized variant thereof. Said consensus CDRs for the VHH3.89 family being more specifically defined as:
CDR1: XYXXG, wherein X (Xaa) at position 1 is D or Y; X (Xaa) at position 3 is D or A, and X (Xaa) at position 4 is V or I (SEQ ID NO: 138). More in particular, CDR1 can be defined as YYAIG (SEQ ID NO: 131; comprised in VHH3.89 and VHH3_183) or DYDVG (SEQ ID NO:132; comprised in VHH3C_80);
CDR2: RIXSSDGSTYYADSVKG, wherein X (Xaa) at position 3 is D or E (SEQ ID NO:139). More in particular, CDR2 can be defined as RIDSSDGSTYYADSVKG (SEQ ID NO:133; comprised in VHH3.89 and VHH3C_80), RIESSDGSTYYADSVKG (SEQ ID NO:134; comprised in VHH3_183);
CDR3: DPIIXGXXWYWT, wherein X (Xaa) at position 5 is R or Q, X (Xaa) at position 7 is R, S or H, and wherein X(Xaa) at position 8 is N or S (SEQ ID NO:140). More in particular, CDR3 can be defined as DPIIQGRNWYWT (SEQ ID NO:135; comprised in VHH3.89), or DPIIQGSSWYWT (SEQ ID NO:136, comprised in VHH3_183), or DPIIRGHNWYWT (SEQ ID NO:137, comprised in VHH3C_80).

In a further specific embodiment, said binding agents or binding domains as defined by the ISVDs comprising the CDRS as listed above, are provided by a functional variant thereof, characterized in that said variant still provides for the same or very similar binding and neutralization properties, and/or a humanized variant thereof, as described herein.

In a further specific embodiment, said binding agent or binding domains comprising a first ISVD or an ISVD specifically binding to binding site 1 as depicted in the VHHs of the VHH72 family, or further VHH families described herein binding to the same epitope, and/or competing with VHH72, and as set forth in SEQ ID NOs:2-21, 90 or SEQ ID NOs:95-98, or a humanized variant thereof which has at least 90% identity with said original VHH sequence as depicted in said SEQ ID NOs:2-21, 90 or SEQ ID NOs:95-98, and wherein the CDRs are identical and said 90% identity is calculated for instance for FR1 of the variant being 90% identical to the entire length of FR1 of the original VHH sequence as depicted in any of SEQ ID NOs:2-21, 90 or SEQ ID Nos:95-98, and FR2 of the variant being 90% identical to the entire length of FR2 of the original VHH sequence as depicted in any of SEQ ID Nos: 2-21, 90 or SEQ ID NOs:95-98, and FR3 of the variant being 90% identical to the entire length of FR3 of the original VHH sequence as depicted in any of SEQ ID NOs:2-21, 90 or SEQ ID NOs:95-98, and FR4 of the variant being 90% identical to the entire length of FR4 of the original VHH sequence as depicted in any of SEQ ID NOs:2-21, 90 or SEQ ID NOs:95-98, and/or alternatively, the CDRs being identical, but the full length sequence being at least 90% identical to the original VHH sequence.

In a further specific embodiment, said binding agent or binding domains comprising a second ISVD or an ISVD specifically binding to binding site 2 as depicted in the VHHs of the VHH3.117 family, or further VHH families described herein binding to the same epitope, and/or competing with VHH3.117, and in a specific embodiment as set forth in SEQ ID Nos:22-27 for the VH3.117 family members, and SEQ ID Nos:85-87 for the VHH3.89 family members, or a humanized variant thereof which has at least 90% identity with said original VHH sequence as depicted in said SEQ ID Nos:22-27 or SEQ ID Nos:85-87, and wherein the CDRs are identical and said 90% identity is calculated for instance for FR1 of the variant being 90% identical to the entire length of FR1 of the original VHH sequence as depicted in any of SEQ ID NOs:22-27 or SEQ ID NOs:85-87, and FR2 of the variant being 90% identical to the entire length of FR2 of the original VHH sequence as depicted in any of SEQ ID NOs:22-27 or SEQ ID NOs:85-87, and FR3 of the variant being 90% identical to the entire length of FR3 of the original VHH sequence as depicted in any of SEQ ID NOs:22-27 or SEQ ID NOs:85-87, and FR4 of the variant being 90% identical to the entire length of FR4 of the original VHH sequence as depicted in any of SEQ ID NOs:22-27 or SEQ ID NOs:85-87, and/or alternatively, the CDRs being identical, but the full length sequence being at least 90% identical to the original VHH sequence.

In a further aspect, the polypeptidic or polypeptide binding agents according to the current invention can be comprising one or more framework regions (FRs) as comprised in any of the ISVDs defined hereinabove.

In a further embodiment, said polypeptidic or polypeptide binding agents binding agents are comprising one or more amino acid sequences with at least 90% identity to an amino acid sequence selected from the group of SEQ ID NOs: 2-27, 90 or SEQ ID NOs:95-98 or SEQ ID NOs:85-87, or with at least 95% identity to an amino acid sequence selected from the group of SEQ ID NOs: 2-27, 90 or SEQ ID NOs:95-98 or SEQ ID NOs:85-87, or with at least 97% identity to an amino acid sequence selected from the group of SEQ ID NOs: 2-27, 90 or SEQ ID NOs:95-98 or SEQ ID NOs:85-87. In particular, such non-identity or variability, is limited to non-identity or variability in FR amino acid residues. In particular, such non-identity or variability may be introduced to obtain a humanized variant of an ISVD defined by or set forth in any of to an amino acid sequence selected from the group of SEQ ID NOs: 2-27, 90 or SEQ ID NOs:95-98 or SEQ ID NOs:85-87. In particular, such humanized variant is a functional orthologue of the original ISVD, wherein the functionality is defined as described herein.

In a particular embodiment, said composition comprises binding agents or binding domains specifically binding to epitope 1 and epitope 2 of the RBD as described herein, or binding agents comprising one or more first ISVDs and one or more seconds ISVDs, wherein said binding agents or binding domains constitute a single or one molecule or agent. In fact, said single binding agent of the composition that is capable of binding to binding site 1 and 2, as defined herein, is a bispecific or biparatopic molecule, or may even be a multi-specific or multiparatopic molecule or agent.

Another embodiment relates to said polypeptidic or polypeptide binding agents that are comprising one or more ISVDs (or variants or humanized forms thereof as described herein) specifically binding epitope 1 and/or epitope 2, in particular one or more first ISVDs and one or more second ISVDs, wherein the at least one or more (first and second) ISVD (or variant or humanized form thereof as described herein) are linked to a bispecific or multispecific agent by direct linking or by fusion via a spacer or linker, such as a peptide linker.

In another embodiment, said polypeptidic or polypeptide binding agents that are comprising one or more ISVDs (or variants or humanized forms thereof as described herein) specifically binding epitope 1 and/or epitope 2, in particular one or more first ISVDs and one or more second ISVDs, the at least one or more (first and second) ISVD (or variant or humanized form thereof as described herein) is bound or fused to an Fc domain, which is defined herein as the fragment crystallizable region (Fc region) of an antibody, which is the tail region known to interact with cell surface receptors called Fc receptors and some proteins of the complement system. Said Fc domain is composed of two identical protein fragments, derived from the second and third constant domains of the antibody's two heavy chains. All conventional antibodies comprise an Fc domain, hence, the Fc domain fusion may comprise an Fc domain derived from or as a variant of the IgG, IgA and IgD antibody Fc regions, even more specifically an IgG1, IgG2 or IgG4. The hinge region of IgG2, may be replaced by the hinge of human IgG1 to generate ISVD fusion constructs, and vice versa. Additional linkers that are used to fuse an ISVD to an Fc domain such as the IgG1 and IgG2 Fc domains comprise (G4S)₂₋₃ linkers, and 20GS linkers. In addition, Fc variants with known half-life extension may be used such as the M257Y/S259T/T261E (also known as YTE) or the LS variant (M428L combined with N434S). These mutations increase the binding of the Fc domain of a conventional antibody to the neonatal receptor (FcRn).

In some embodiments, the Fc region is engineered to create “knobs” and “holes” which facilitate heterodimer formation of two different Fc-containing polypeptide chains when co-expressed in a cell (U.S. Pat. No. 7,695,963). The Fc region may be altered to use electrostatic steering to encourage heterodimer formation while discouraging homodimer formation of two different Fc-containing polypeptide when co-expressed in a cell (WO 09/089,004).

The term “knob-into-hole” or “KiH” technology as mentioned herein generally refers to the technology directing the pairing of two polypeptides together in vitro or in vivo by introducing a protuberance (knob) into one polypeptide and a cavity (hole) into the other polypeptide at an interface in which they interact. A “protuberance” or “knob” may refer to at least one amino acid side chain which projects from the interface of a first polypeptide and is therefore positionable in a compensatory cavity or hole in the adjacent interface (i.e. the interface of a second polypeptide) so as to stabilize the heteromultimer, and thereby favour heteromultimer formation over homomultimer formation, for example. The use of knobs into holes as a method of producing multispecific antibodies is well known in the art. Multispecific antibodies having KiH in their Fc domain can further comprise one or more different ISVDs linked to each Fc domain.

In a particular further embodiment, the polypeptidic or polypeptide binding agents of the invention comprising one or more ISVDs, in particular one or more first ISVDs and one or more second ISVDs (or variants or humanized forms thereof as described herein), are in a “multivalent” or “multispecific” form and are formed by bonding, chemically or by recombinant DNA techniques, together two or more identical or variant monovalent ISVDs (or variants or humanized forms thereof as described herein). Said multivalent forms may be formed by connecting the building block directly or via a linker, or through fusing the building blocks with an Fc domain encoding sequence. Non-limiting examples of multivalent constructs include “bivalent” constructs, “trivalent” constructs, “tetravalent” constructs, and so on. The ISVDs (or variants or humanized forms thereof as described herein) comprised within a multivalent construct may be identical or different.

In another particular embodiment, the ISVDs (or variants or humanized forms thereof as described herein) of the invention are in a “multi-specific” form, format or construct and are formed by bonding together two or more ISVDs, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) ISVD of the invention may be directed against two or more of the same antigens, such as the spike protein, but binding non-competing or different epitopes of binding sites, such as binding site 1 and 2 as defined herein, providing for biparatopic agents, or alternatively may be directed against different antigens, for example against the Corona RBD and one as a half-life extension against Serum Albumin or SpA. Multivalent or multi-specific ISVDs of the invention may also have (or be engineered and/or selected for) increased avidity and/or improved selectivity for the desired Corona RBD interaction, and/or for any other desired property or combination of desired properties that may be obtained by the use of such multivalent or multi-specific immunoglobulin single variable domains.

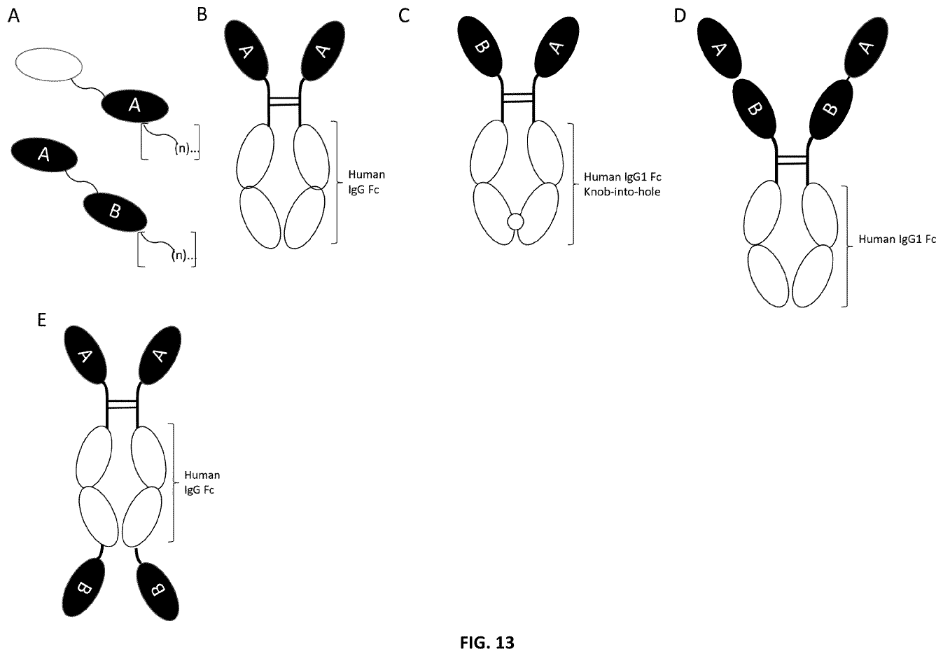

In particular embodiments, the one or more first ISVDs are linked, fused or connected directly or via a linker to the one or more second ISVDs, preferably the binding agent comprises one first ISVD linked, fused or connected directly or via a linker to one second ISVD. Non-limiting examples of suitable linkers for linking the ISVDs include peptide linkers such as a (G4S)ₙ, wherein n=1, 2, 3, 4, 5 or 6. A schematic drawing of such multispecific binding agents, also referred to herein as “head-to-tail fusions”, is depicted in FIG. 13A. More specific examples of such multispecific binding agents, in particular bispecific binding agents, which are capable of binding the 2 conserved binding sites as described herein, are provided in for instance, but not limited to, Table 3, and SEQ ID NOs: 76-93, or any functional variant thereof, or a variant with at least 90% identity thereof, or a humanized variant thereof.

In further embodiments, the C-terminus of such a bivalent or bispecific binding agent may be fused, e.g. by a linker, to an Fc domain, which construct upon expression in a host forms a multivalent or multispecific binding agent, in particular a tetravalent bispecific binding agent, through disulfide bridges in the hinge region of the Fc part. Accordingly, in particular embodiments, the one or more first ISVDs are linked, fused or connected directly or via a linker to the one or more second ISVDs to form a multispecific binding agents and said multispecific binding agent is fused to an Fc domain. In preferred embodiments, the binding agent comprises a bispecific binding agent fused to an Fc domain, wherein said bispecific binding agent comprises one first ISVD linked, fused or connected directly or via a linker to one second ISVD. A schematic drawing of such multispecific binding agents, also referred to herein as “VHH-VHH-Fc fusion”, is depicted in FIG. 13D. More specific examples of such multispecific binding agents, in particular bispecific binding agents, which are capable of binding the 2 conserved binding sites as described herein, are provided in for instance, but not limited to, Table 9, and SEQ ID NO:118, or any functional variant thereof, or a variant with at least 90% identity thereof, or a humanized variant thereof.

In other particular embodiments, the one or more first ISVDs are fused to the N-terminus of an Fc domain, and the one or more second ISVDs are fused to the C-terminus of the Fc domain, or the one or more first ISVDs are fused to the C-terminus of an Fc domain, and the one or more second ISVDs are fused to the N-terminus of the Fc domain. In preferred embodiments, the binding agent comprises one first ISVD fused to the N-terminus of an Fc domain and one second ISVD fused to the C-terminus of the Fc domain, or the one first ISVD is fused to the C-terminus of the Fc domain and the one second ISVD is fused to the N-terminus of the Fc domain. A schematic drawing of such multispecific binding agents, also referred to herein as “VHH-Fc-VHH fusions” or “moonlanders”, is depicted in FIG. 13E. More specific examples of such multispecific binding agents, in particular bispecific binding agents, which are capable of binding the 2 conserved binding sites as described herein, are provided in for instance, but not limited to, Table 10, and SEQ ID NOs:119-121, or any functional variant thereof, or a variant with at least 90% identity thereof, or a humanized variant thereof.

In particular embodiments, the one or more first ISVDs are fused to an Fc domain comprising a knob, and the one or more second ISVDs are fused to an Fc domain comprising a hole, or, the one or more first ISVDs are fused to an Fc domain comprising a hole, and the one or more second ISVDs are fused to an Fc domain comprising a knob. A schematic drawing of such multispecific binding agents, also referred to herein as “knob-into-holes” or “KiHs” or “knob-into-hole VHH-Fc fusions”, is depicted in FIG. 13C. More specific examples of such multispecific binding agents, in particular bispecific binding agents, which are capable of binding the 2 conserved binding sites as described herein, are provided in for instance, but not limited to, Table 8, and knob-in-hole sequence pairs SEQ ID NOs: 107/108, 109/110, 111/112, 113/114, 115/116 and 113/117, or any functional variant thereof, or a variant with at least 90% identity thereof, or a humanized variant thereof. In another embodiment, the invention provides a composition comprising at least two polypeptidic or polypeptide binding agents specifically binding epitope 1 and 2, resp., or comprising a single binding agent specifically binding both sites, epitope 1 and 2, wherein the paratopes for epitopes 1 and 2 are constituted by amino acids of ISVDs (or variants or humanized forms thereof as described herein) according to the invention, either in a monovalent, multivalent or multi-specific form. Thus, monovalent, multivalent or multi-specific polypeptidic or polypeptide binding agents comprising a herein described ISVD (or variant or humanized form thereof as described herein) or part thereof are included here as non-limiting examples.

Particularly, a single ISVD (or variant or humanized form thereof) as described herein, such as a first ISVD or a second ISVD as described herein, may be fused at its C-terminus to an IgG Fc domain, resulting in a sarbecovirus binding agents of bivalent format wherein two of said ISVDs (or variants or humanized forms thereof as described herein), form a heavy chain only-antibody-type molecule through disulfide bridges in the hinge region of the IgG Fc part. Said humanized forms thereof, include but are not limited to the IgG humanization variants known in the art, such as C-terminal deletion of Lysine, alteration or truncation in the hinge region, LALA or LALAPG mutations (Schlothauer, et al. 2016, Protein Eng. Des. Sel. PEDS 29, 457-466) as described herein, among other substitutions in the IgG sequence.

A further specific embodiment thus relates to said compositions comprising any of said binding agents, more specifically polypeptidic bispecific or tetravalent bispecific agents as disclosed herein, which are constituting at least one binder for epitope 1, such as a first ISVD as described herein such as the ISVDs depicted by VHH72 (or a variant thereof), or VHH3.83 (or a variant thereof), and at least one binder for epitope 2, such as a second ISVD as described herein such as the ISVDs depicted by VHH3.117 (or a variant thereof), or VHH3.89 (or a variant thereof). More specific examples of such composition comprising said multispecific binding agents specifically binding said 2 conserved epitopes are provided in for instance, but not limited to, Table 3, and SEQ ID NOs: 76-84 and 91-93, or any functional variant thereof, or a variant with at least 90% identity thereof, or a humanized variant thereof.

In yet another aspect, the invention provides nucleic acid molecules such as isolated nucleic acids, (isolated) chimeric gene constructs, expression cassettes, recombinant vectors (such as expression or cloning vectors) comprising a nucleotide sequence, such a coding sequence, that is encoding the polypeptide binding agent or binding domain or single binding agent as identified herein.

One further aspect of the invention provides for a host cell comprising the composition or the binding agent(s) or binding domains or part thereof, such as an ISVD or part thereof, as described herein. The host cell may therefore comprise the nucleic acid molecule encoding said polypeptide binding agent or binding domain. Host cells can be either prokaryotic or eukaryotic. The host cell may also be a recombinant host cell, which involves a cell which has been genetically modified to contain an isolated DNA molecule, nucleic acid molecule encoding the polypeptide binding agent of the invention. Representative host cells that may be used to produce said ISVDs, but are not limited to, bacterial cells, yeast cells, plant cells and animal cells. Bacterial host cells suitable for production of the binding agents of the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis, Virology Volume 310, Issue 1, 25 May 2003, Pages 1-7). Alternatively, the host cells may also be transgenic animals or plants.

A further aspect of the invention relates to medicaments or pharmaceutical compositions comprising the binding agent(s) and/or nucleic acid encoding it, and/or a recombinant vector comprising the nucleic acid, as described herein. In particular, a pharmaceutical composition is a pharmaceutically acceptable composition; such compositions are in a particular embodiment further comprising a (pharmaceutically) suitable or acceptable carrier, diluent, stabilizer, etc.

A further aspect of the invention relates to a composition comprising said one or more binding agents or binding domains specifically binding epitope 1 and 2 of the spike protein ads described herein, the nucleic acid encoding it as described herein, or to a pharmaceutical composition comprising a binding agent, nucleic acid encoding it, and/or a recombinant vector comprising such nucleic acid, as described herein, for use as a medicine or medicament.

Alternatively, use of said composition, binding agent or nucleic acid encoding it as described herein, or use of a pharmaceutical composition comprising a binding agent, nucleic acid encoding it, and/or a recombinant vector comprising such nucleic acid, as described herein, in the manufacture of a medicine or medicament is envisaged. In particular, the composition, binding agent or nucleic acid encoding it as described herein, or the medicament or pharmaceutical composition comprising a binding agent, nucleic acid encoding it, and/or a recombinant vector comprising such nucleic acid, as described herein, is for use in passive immunisation, for use in treating a subject with a sarbecovirus infection, for use in preventing infection of a subject with a sarbecovirus, or for use in protecting a subject from infection with a sarbecovirus. When for use in passive immunisation, the subject may have an infection with a sarbecovirus (therapeutic passive immunisation) or may not have an infection with a sarbecovirus (prophylactic passive immunisation).

A further aspect of the invention relates to methods for treating a subject suffering from/having/that has contracted an infection with a sarbecovirus, the methods comprising administering a composition comprising said binding agent(s) or nucleic acid encoding it as described herein to the subject, or comprising administering a medicament or pharmaceutical composition comprising a binding agent or nucleic acid encoding it as described herein to the subject.

A further aspect of the invention relates to methods for protecting a subject from infection with a sarbecovirus or for preventing infection of a subject with a sarbecovirus, the methods comprising administering a composition or binding agent or nucleic acid encoding it as described herein to the subject prior to infection, or comprising administering a medicament or pharmaceutical composition comprising a binding agent or nucleic acid encoding it as described herein to the subject prior to infection.

In particular, in the above medical aspects, the sarbecovirus is a coronavirus, more in particular a zoonotic coronavirus, even more in particular SARS-COV-2 or SARS-COV-1, even more in particular SARS-CoV-2 variants such as variants at position N439, S477, E484, N501 or D614 (relative to the SARS-COV-2 spike amino acid sequence as defined in SEQ ID NO:1). In particular, treatment is referring to passive immunisation of a subject having contracted a sarbecovirus infection. In particular, prevention of infection with a sarbecovirus is useful in case of e.g. epidemic or pandemic conditions during which subjects known to be most vulnerable to develop severe disease symptoms can be prophylactically treated (preventive or prophylactic immunisation) with a composition or binding agent or nucleic acid encoding it as described herein such as to prevent infection overall, or such as to prevent development or occurrence of severe disease symptoms. In order to achieve the preventive or prophylactic effect, the composition or binding agent or nucleic acid encoding it as described herein may need to be administered to a subject multiple times, such as with an interval of 1 week or 2 weeks; the interval being dictated by the pharmacokinetic behaviour or characteristics (half-time) of the binding agent or nucleic acid. Further in particular, the subject is a mammal susceptible to infection with the sarbecovirus, such as a human subject that is susceptible to infection with SARS-COV-2 or SARS-COV-1. Furthermore, in particular to the above medical aspects, a nucleic acid encoding a binding agent as described herein can be used in e.g. gene therapy setting or RNA vaccination setting.

A further specific embodiment relates to prophylactic treatment, in which a single dose of a (pharmaceutical) composition or binding agent as described herein is administered and wherein the single dose is in the range of 0.5 mg/kg to 25 mg/kg. Alternatively, a therapeutic treatment with a (pharmaceutical) composition or binding agent is envisaged wherein a single dose in the range of 0.5 mg/kg to 25 mg/kg is envisaged. In both prophylactic and therapeutic settings, multiple doses may need to be administered, and the time interval between two subsequent doses being determined by the half-life of the composition or binding agent in the subject's circulation.

Furthermore in particular to the above medical aspects, the composition, binding agent, nucleic acid or pharmaceutical composition may be administered to a subject via intravenous injection, subcutaneous injection, or intranasally, or, alternatively via inhalation or pulmonary delivery.

Furthermore, in particular to the above medical aspects, a therapeutically effective amount of the composition or binding agent, nucleic acid or pharmaceutical composition is administered to a subject in need thereof; the administration of such therapeutically effective amount leading to inhibiting or preventing infection with a sarbecovirus, and/or leading to curing infection with a sarbecovirus.

A further aspect of the invention relates to a composition or binding agent as described herein for use in diagnosing a sarbecovirus infection, for use as a diagnostic agent, or for use in the manufacture of a diagnostic agent or diagnostic kit, such as an in vitro diagnostic agent or kit. Alternatively, use of a composition or binding agent as described herein in the manufacture of a diagnostic agent/in vitro diagnostic agent is envisaged. In particular, the composition or binding agent as described herein is for use in detecting the presence (or absence) of a sarbecovirus in a sample, such as a sample obtained from a subject, such as from a subject suspected to be infected with a sarbecovirus infection. A nucleic acid encoding a binding agent or pan-specific binding agent as described herein, or a recombinant vector comprising such nucleic acid can likewise be used in or be for use in the manufacture of a diagnostic agent or diagnostic kit, such as an in vitro diagnostic agent or kit.

A further aspect of the invention relates to methods for detecting a sarbecovirus in a sample, such as a sample obtained from a subject, such as from a subject suspected to be infected with a sarbecovirus infection. Such methods usually comprise the steps of obtaining a sample, contacting the sample with a composition or binding agent as described herein, and detecting, determining, assessing, assaying, identifying or measuring binding of the binding agent with a sarbecovirus.

In particular, in the above diagnostic aspects, the sarbecovirus is a coronavirus, more in particular a zoonotic coronavirus, even more in particular SARS-COV-2 or SARS-COV-1. Further in particular, the subject is a mammal susceptible to infection with the sarbecovirus, such as a human subject that is susceptible to infection with SARS-COV-2 or SARS-COV-1.

Further in particular, in the above diagnostic aspects, the composition or binding agent as described herein is comprising a detectable moiety fused to it, bound to it, coupled to it, linked to it, complexed to it, or chelated to it. A “detectable moiety” in general refers to a moiety that emits a signal or is capable of emitting a signal upon adequate stimulation, or to a moiety that is capable of being detected through binding or interaction with a further molecule (e.g. a tag, such as an affinity tag, that is specifically recognized by a labelled antibody), or is detectable by any means (preferably by a non-invasive means, if detection is in vivo/inside the human body). Furthermore, the detectable moiety may allow for computerized composition of an image, as such the detectable moiety may be called an imaging agent. Detectable moieties include fluorescence emitters, phosphorescence emitters, positron emitters, radioemitters, etc., but are not limited to emitters as such moieties also include enzymes (capable of measurably converting a substrate) and molecular tags. Examples of radioemitters/radiolabels include ⁶⁸Ga, ^{110m}In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²CI, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga. Fluorescence emitters include cyanine dyes (e.g. Cy5, Cy5.5, Cy7, Cy7.5), FITC, TRITC, coumarin, indolenine-based dyes, benzoindolenine-based dyes, phenoxazines, BODIPY dyes, rhodamines, Si-rhodamines, Alexa dyes, and derivatives of any thereof. Affinity tags, such as chitin binding protein (CBP), maltose binding protein (MBP), glutathione-S-transferase (GST), poly(His) (e.g., 6×His or His6), biotin or streptavidin, such as Strep-Tag®, Strep-tag Il® and Twin-Strep-Tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase).

Compositions or Binding agents as describe herein and comprising a detectable moiety may for example be used for in vitro, in vivo or in situ assays (including immunoassays known per se such as ELISA, RIA, EIA and other “sandwich assays”, etc.) as well as in vivo imaging purposes, depending on the choice of the specific label. A specific embodiment discloses the use of the composition or binding agent, optionally in a labelled form, for detection of a virus or Spike protein of said virus, wherein said virus is selected from the group of clade 1a, 1b, 2 and/or clade 3 bat SARS-related sarbecovirsues, such as SARS-Cov-2, GD-Pangolin, RaTG13, WIV1, LYRa11, RsSHC014, Rs7327, SARS-COV-1, Rs4231, Rs4084, Rp3, HKU3-1, or BM48-31 viruses.

In another alternative aspect of the invention, any of the compositions or binding agents described herein, optionally with a label, or any of the nucleic acid molecules encoding said agent, or any of the pharmaceutical compositions, or vectors as described herein may as well be used as a diagnostic, or in detection of a corona virus, as described herein. Diagnostic methods are known to the skilled person and may involve biological samples from a subject. Also in vitro methods may be in scope for detection of viral protein or particles using the binding agents as described herein. Finally, the composition or binding agents as described herein, optionally labelled, may also be suitable for use in in vivo imaging.

A further aspect of the invention relates to kits comprising a composition or binding agent or nucleic acid encoding it as described herein, or a pharmaceutical composition comprising a binding agent or nucleic acid encoding it as described herein.

Such kits comprise pharmaceutical kits or medicament kits which are comprising a container or vial (any suitable container or vial, such as a pharmaceutically acceptable container or vial) comprising an amount of binding agent or nucleic acid encoding it as described herein, and further comprising e.g. a kit insert such as a medical leaflet or package leaflet comprising information on e.g. intended indications (prophylactic or therapeutic treatment of sarbecovirus infection) and potential side-effects. Pharmaceutical kits or medicament kits may further comprise e.g. a syringe for administering the binding agent or nucleic acid encoding it as described herein to a subject.

Such kits comprise diagnostic kits comprising a container or vial (any suitable container or vial, such as a pharmaceutically acceptable container or vial) comprising an amount of binding agent as described herein, such as a binding agent comprising a detectable moiety. Such diagnostic kits may further comprise e.g. one or more reagents to detect the detectable moiety and/or e.g. instructions on how to use said binding agent for detection of a sarbecovirus in a sample.

Certain aspects and embodiments of the present invention are set forth in the below numbered statements:

(1) A composition containing one or more binding agents or binding domains, which specifically bind the Corona virus Spike protein RBD at a first binding site which comprises at least one of the amino acid residues Y369, F377, and K378 of the SARS-COV-2 spike protein as defined by SEQ ID NO:1, and at a second binding site comprising at least one or more of the amino acids T393, N394, V395, or Y396 of the SARS-COV-2 spike protein as defined by SEQ ID NO:1. In fact said first and second binding sites may also be defined as the minimal residues needed for a binding agent or binding domain to specifically interact with VHH72 (Wrap et al. 2020; Cell 184:1004-1015; PCT/EP2021/052885) and VHH3.117 (as shown herein, and EP21166835.5 and PCT/EP2022/052919), respectively. Said first and second binding site provide for a dual binding region each of which separately allow for neutralization of SARS-COV-1 and SARS-COV-2 viruses, and providing for a binding region on the RBD domain of the corona virus spike protein that is conserved among the sarbecoviruses, and thus less prone to mutation and escape from neutralization.

(2) A composition comprising one or more agents specifically binding the Corona virus Spike protein to a binding site 1 comprising the amino acid residues Y369, F377, and K378, and a binding site 2 comprising at least one or more of the residues T393, N394, V395, or Y396 of the SARS-COV-2 spike protein as depicted in SEQ ID NO:1.

(3) The composition of (1) or (2), wherein binding site 1 further comprises at least one or more of the amino acid residues of L368, S371, S375, T376, C379 and/or Y508 of the SARS-COV-2 spike protein as depicted in SEQ ID NO:1.

(4) The composition of any one of (1) to (3), wherein binding site 2 further comprises at least one or more of residues R357, K462, F464, E465, R466, S514, E516, or L518 of the SARS-COV-2 spike protein as depicted in SEQ ID NO:1. In a specific embodiment, the composition comprises at least one binding agent or binding domain specifically binding the first binding site, comprising at least one or more or all of the residues Y369, F377, K378, L368, S371, S375, T376, C379 and/or Y508 of the SARS-COV-2 spike protein provided in SEQ ID NO:1, and one or more binding agents or binding domains specifically binding the second binding site, comprising at least on or more residues of T393, N394, V395, Y396, R357, K462, F464, E465, R466, S514, E516, or L518 of the SARS-COV-2 spike protein as depicted in SEQ ID NO:1. Said binding agent of domain specific for binding site 1 may be part of the same molecule as the binding agent or domain specific for binding site 2 of the RBD, or may be part of a different molecule.

In a further specific embodiment, the binding agent of the composition specifically binding to binding site 1 of the RBD as defined herein, provides for an agent that competes for binding to the RBD, with the Angiotensin-Converting Enzyme 2 (ACE2), and is capable of at least neutralizing SARS-COV-2 and/or SARS-COV-1 when bound to binding site 1 of the spike protein, as defined herein. In a further specific embodiment, the binding agent of the composition specifically binding to binding site 2 of the RBD as defined herein, provides for an agent that allows ACE2 to bind to the RBD, and is capable of at least neutralizing SARS-COV-2 and/or SARS-COV-1 when the binding agent is bound to binding site 2 of the RBD as defined herein. In a further specific embodiment, the binding agent comprises two binding regions, preferably ISVDs, capable of specifically binding to binding site 1 and binding site 2 of the RBD as defined herein, resp., and thus provides for an agent that competes with the Angiotensin-Converting Enzyme 2 (ACE2) for binding to the RBD (via its binding to binding site 1), and is capable of at least neutralizing SARS-COV-2 and SARS-COV-1 when bound to binding site 1 and/or 2 as defined herein. Furthermore, the binding agent specifically binding site 2, as defined herein, may allow binding of ACE2, as well as of antibodies VHH72, S309, or CB6 to SPRBD when bound to SPRBD.

(5) The composition of any one of (1) to (4), wherein the one or more agents comprise one or more immunoglobulin single variable domains (ISVDs), preferably wherein said one or more ISVD(s) specifically bind(s) the first and/or second binding site. The composition may comprise at least two binding agents or binding domains, at least one specifically binding to binding site 1 of the spike protein of SEQ ID NO:1, as defined herein, and at least one specifically binding to binding site 2 of the spike protein of SEQ ID NO:1, as defined herein. Alternatively, the composition may comprise a single binding agent that is capable of binding both, via a first and second binding domain, to the first and second binding site of the spike protein, respectively, as defined herein. Bindings agent capable of binding both binding sites of the RBD as defined herein, may comprise at least two ISVDs, of which one specifically binds the first and one specifically binds the second binding site on the RBD, as defined herein.

(6) The composition of (5), wherein the one or more binding agents comprise an ISVD specifically binding site 1, comprise the complementarity determining regions (CDRs) as depicted in any of SEQ ID NOs: 2-21 and SEQ ID NOs: 95-98, wherein the CDRs are annotated according to Kabat, MacCallum, IMGT, AbM, aHo, Chothia, Gelfand, or Honegger, or wherein CDR1 is defined by any of SEQ ID NOs: 28-37, CDR2 is defined by any of SEQ ID NOs: 38-50, and CDR3 is defined by any of SEQ ID NOS: 51-61. VHH's defined by SEQ ID NOs: 2-21 or SEQ ID NOs: 95-98 specifically bind the first RBD epitope comprising at least one or more residues of Y369, F377, and K378 of SEQ ID NO:1, or more specifically additionally bind one or more residues of L368, S371, S375, T376, C379 and/or Y508 of SEQ ID NO:1.

(7) The composition of (5), wherein the one or more binding agents comprise an ISVD specifically binding site 2, comprise the complementarity determining regions (CDRs) present in any of SEQ ID NOs: 22-27, wherein the CDRs are annotated according to Kabat, MacCallum, IMGT, AbM, aHo, Chothia, Gelfand, or Honegger, or wherein CDR1 is defined by SEQ ID NO: 70, wherein X (Xaa) at position 2 is S (Ser, serine) or N (Asn, asparagine), CDR2 is defined by SEQ ID NO:71 wherein X (Xaa) at position 5 is T (Thr, threonine) or S (Ser, serine), X (Xaa) at position 7 is S (Ser, serine) or N (Asn, asparagine), X (Xaa) at position 12 is D (Asp, aspartic acid) or N (Asn, asparagine), X (Xaa) at position 14 is A (Ala, alanine) or V (Val, valine), and X (Xaa) at 15 position 15 is Q (Gln, glutamine) or K (Lys, lysine); and CDR3 is defined by SEQ ID NO:72, wherein X (Xaa) at position 3 is P (Pro, proline) or L (Leu, leucine); or CDR1 is defined by any of SEQ ID NO: 62 or 63, CDR2 is defined by any of SEQ ID NO: 64-67 and CDR3 is defined by any of SEQ ID NO: 68 or 69. VHH's defined by SEQ ID NOs: 22-27 specifically bind the second RBD epitope comprising at least one or more residues of T393, N394, V395, or Y396 of SEQ ID NO:1, or more specifically additionally bind one or more residues of R357, K462, F464, E465, R466, S514, E516, or L518 of SEQ ID NO:1.

(8) The composition of (5), wherein the one or more binding agents comprise an ISVD specifically binding site 2, comprise the complementarity determining regions (CDRs) present in any of SEQ ID NOs: 85-87, wherein the CDRs are annotated according to Kabat, MacCallum, IMGT, AbM, aHo, Chothia, Gelfand, or Honegger.

(9) The composition of any one of (5) to (8), wherein the one or more binding agents comprising an ISVD specifically binding site 1, comprise a sequence selected from the group of sequences of SEQ ID NOs: 2-21 and SEQ ID NOs: 95-98, or a functional variant with at least 90% identity thereof wherein the non-identical amino acids are located in one or more FRs, or a humanized variant thereof. In a specific embodiment, said composition comprising one or more binding agents or domains comprising an ISVD specifically binding site 1, as defined herein, comprise the complementarity determining regions (CDRs) as depicted in any of the VHHs identified as VHH72, or a variant thereof, or as provided by SEQ ID NOs: 2-5, or SEQ ID NO: 90 herein, or a VHH72 family member, as provided by SEQ ID NO: 9-14, or a humanized variant of any one thereof. In a specific embodiment, said composition comprising one or more binding agents comprising an ISVD specifically binding site 1, as defined herein, comprise the complementarity determining regions (CDRs) as depicted in any of the VHHs identified as VHH3.83, or a variant thereof, or as defined by SEQ ID NOs: 6-8 herein, or a family member of VHH3.83, as provided by VHH4.1XAS51, VHH4.2XAS58, VHH4.2XAS31, and VHH4.2XAS43, and as depicted in SEQ ID NO:95-98, respectively, or as depicted in any of the VHHs identified as VHH3.55, or a variant thereof, or as defined by SEQ ID NOs: 17 herein, or a VHH3.55 family member, or as defined by VHH3.35 depicted in SEQ ID NO: 18, or a humanized variant of any one thereof. In a specific embodiment, said composition comprising one or more binding agents comprising an ISVD which competes for binding to the sarbecovirus RBD with VHH72, as defined herein, comprising the CDRs as depicted in any of the VHHs identified as VHH3.36, VHH3.47, VHH3.29 or VHH3.149, or a variant thereof, or as defined by SEQ ID NOs: 15, 16, 19 or 21, respectively, or a humanized variant of any one thereof.
(10) The composition of any one of (5) to (9), wherein the one or more binding agents comprising an ISVD specifically binding site 2, comprise a sequence selected from the group of sequences of SEQ ID NO: 22-27 and SEQ ID NOs: 85-87 or a functional variant with at least 90% identity thereof wherein the non-identical amino acids are located in one or more FRs, or a humanized variant thereof.
(11) The composition of any one of (1) to (10), comprising at least one agent that competes with any of the binding agents selected from the group of SEQ ID NO: 2-21 and SEQ ID NOs: 95-98 for its binding to the RBD, and/or comprising at least one agent that competes with any of the binding agents selected from the group of SEQ ID NO: 22-27 and SEQ ID NOs: 85-87 for its binding to the RBD.
(12) The composition of any one of (1) to (11), comprising a single agent specifically binding to binding site 1 and binding site 2 of the Spike protein. Specifically, said composition comprises a single binding agent or molecule comprising one or more immunoglobulin single variable domains (ISVDs), wherein said one or more ISVD specifically bind the first and/or second binding site. Said binding agent comprising an ISVD specifically binding to binding site 1 and/or comprising an ISVD specifically binding to binding site 2, as defined herein, is obtained by a fusion of said binders, wherein said fusion may be directly made by linking the binding domains, or wherein said fusion is made via a linker, or via another moiety, as further specified herein. Said linker may be a peptide linker of one or more amino acid residues, or may be another protein or antibody portion, such as a heavy chain Fc-tail or another moiety. In a specific embodiment, said binding agent comprises an Fc tail, which is fused to at least one of said binding site 1 and/or binding site 2 binders, wherein said Fc is preferably derived from an IgG. So, by expressing said Fc tail fused to at least one of said binding agents specific for binding site 1 and Fc tail fused to at least one of said binding agents specific for binding site 2, a bispecific or biparatopic antibody is formed through the dimerization at the Fc hinge region, which renders a multivalent binding agent, such as a bivalent or tetravalent agent. In a specific embodiment, said composition comprises such a multivalent or multispecific agent specifically binding epitope 1 and 2 of the RBD of the corona virus spike protein as defined herein, which may comprise an ISVD as provided in any of the sequences of SEQ ID NOs: 2-27, SEQ ID NOs: 95-98, SEQ ID NOS: 85-87, or any of the sequences selected from the group of SEQ ID NO: 76-84, or SEQ ID NOs: 91-93, or a functional variant of any one thereof with at least 90% identity thereof, specifically at least 90% identity for each framework region as compared to the original FR sequence, and identical CDRs, and/or a humanized variant of any one thereof.
(13) The composition of (12), wherein said agent comprises an ISVD specifically binding to binding site 1 and comprises an ISVD specifically binding to binding site 2, wherein said ISVDs are fused directly or via a linker.
(14) The composition of (13), wherein said linker may be a short peptide linker or an Fc-tail or another moiety.
(15) The composition of any (12) or (13), wherein said agent comprises an IgG Fc to fuse said ISVDs specific for binding site 1 and 2 providing for a bispecific antibody, wherein said bispecific antibody may be bivalent or tetravalent.
(16) The composition of (11) to (14), wherein said agent comprises a sequence selected from the group of SEQ ID NOs: 76-84 and SEQ ID NOs: 91-93, or a functional variant with at least 90% identity thereof, or a humanized variant of any one thereof.
(17) An isolated nucleic acid encoding a binding agent according to any one of (11) to (16).
(18) A recombinant vector comprising the nucleic acid according to (17).
(19) A pharmaceutical composition comprising the composition according to any one of (1) to (16), an isolated nucleic acid according to (17) and/or a recombinant vector according to (18).
(20) The composition according to any one of (1) to (16), the isolated nucleic acid according to (17), the recombinant vector according to (18), or the pharmaceutical composition according to (19), for use as a medicament.
(21) The composition according to any one of (1) to (16), the isolated nucleic acid according to (17), the recombinant vector according to (18), or the pharmaceutical composition according to (19), for use in passive immunisation of a subject.
(22) The composition according to any one of (1) to (16), the isolated nucleic acid according to (17), the recombinant vector according to (18), or the pharmaceutical composition according to (19), for use in the treatment of a coronavirus infection, more specifically a sarbecovirus infection.
(23) The composition according to any one of (1) to (16), the isolated nucleic acid according to (17), the recombinant vector according to (18), or the pharmaceutical composition according to (19), for use in treatment of SARS-COV-1 or SARS-COV-2 infection.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. VHHs Potently Neutralizing SARS-COV-1 and -2 Via Specific Binding to a Conserved Epitope of the RBD Via Inhibition of the ACE2 Receptor Binding to the Spike Protein

The identification of VHH72 and derived (mutant) variants including Fc fusions thereof has previously been reported (Wrapp et al. Schepens al. 2020; et 2021 Biorxiv doi: https://doi.org/10.1101/2021.03.08.433449). Moreover, the epitope of VHH72 on the RBD of the SARS-CoV1 Spike protein has been disclosed herein. In addition, further SARS-COV-2 neutralizing VHHs from the same VHH72 Nb family, and/or binding to the same epitope, and/or competing with VHH72 have been identified as potently neutralizing SARS-COV-2 by interacting with its Spike protein. These second and third generation VHHs have previously been purified and tested for their capacity to compete with VHH72 for binding to SARS-COV-2 RBD as assessed by AlphaLISA (amplified luminescent proximity homogeneous assay), and/or structurally analyzed in complex with the Spike protein to confirm that the binding site is identical to the VHH72 binding site. These competition and structural analysis data have been reported in Schepens et al. (PCT/EP2021/052885) and provide the basis to classify those VHHs as binders to ‘binding site 1’ or ‘epitope 1’ (or ‘the VHH72-epitope’) as described herein. Selected clones representing different VHH families were recloned for production in either Pichia pastoris or E. coli for further characterization as purified monovalent proteins. Monovalent VHHs contained a C-terminal His6 tag, or C-terminal HA-His6 tag, respectively. Purification was done using Ni-NTA affinity chromatography.

To test the VHHs in AlphaLISA, serial dilutions of anti-SARS-COV-2 VHHs and irrelevant control VHH (final concentration ranging between 90 nM-0.04 nM) were made in assay buffer (PBS containing 0.5% BSA and 0.05% Tween-20). VHHs were subsequently mixed with VHH72-h1 (S65A)-Flag3-His6 (final concentration 0.6 nM) and SARS-COV-2 RBD protein Avi-tag biotinylated (AcroBiosystems, Cat nr. SPD-C82E9) (final concentration 0.5 nM) in white low binding 384-well microtitre plates (F-bottom, Greiner Cat nr 781904). After an incubation for 1 hour at room temperature, donor and acceptor beads were added to a final concentration of 20 μg/mL for each in a final volume of 0,025 mL. Biotinylated RBD was captured on streptavidin coated Alpha Donor beads (Perkin Elmer, Cat nr. 6760002), and VHH72_h1(S56A)-Flag3-His6 was captured on anti-Flag AlphaLISA acceptor beads (Perkin Elmer, Cat nr. AL112C) in an incubation of 1 hour at room temperature in the dark. Binding of VHH72 and RBD captured on the beads leads to an energy transfer from one bead to the other, assessed after illumination at 680 nm and reading at 615 nm of on an Ensight instrument. Results are shown in the FIG. 2. Potencies as determined by IC50 values are shown in Table 1. Results indicate that 7 VHHs (families F-36/55/29/38/149) that are part of a superfamily, and VHH3.83 (Family 83) fully block the interaction of VHH72 to the SARS-COV-2 RBD protein, indicating they bind to at least overlapping or the same epitope as VHH72. Dose-dependent inhibition of the interaction of SARS-COV-2 RBD protein with the ACE-2 receptor was assessed in a competition AlphaLISA, using recombinant human ACE-2-Fc (final concentration 0.2 nM). All VHHs that competed with VHH72 also block the interaction of human ACE2 to the SARS-COV-2 RBD protein (data not shown; PCT/EP2021/052885). With exception of VHH3.83, that showed partial blockade (75% inhibition), all others showed full blockade of ACE-2 binding.

In conclusion, the competition assay confirms that purified VHHs from families F-83, 36, 55, 29, 38 and 149 bind to the same and/or competing epitope of VHH72, and compete with ACE-2 binding similar to the VHH72 family members. The most potent competitors not belonging to the VHH72 family are VHH3.36 and VHH3.83, respectively (Table 1).

TABLE 1
Inhibition of VHH72 (h1 S56A) or ACE2 binding to the SARS-COV-2
RBD by additional anti-SARS-CoV-2 VHHs of the VHH72 family and
of different VHH families, as determined in competition AlphaLISA.

| | | Competition | | Competition | |
|---|---|---|---|---|---|
| | | VHH72/RBD | | ACE2/RBD | |
| VHH | | | % | | % |
| Family | ID | IC50 (M) | inhibition | IC50 (M) | inhibition |
| 72 | VHH72 | 1.60E−08 | 107 | 2.67E−08 | 97 |
| | VHH2.50 | 2.77E−08 | 110 | 3.42E−08 | 79 |
| | VHH3.17 | 3.16E−10 | 99 | 1.03E−09 | 99 |
| | VHH3.77 | 2.37E−10 | 102 | 7.23E−10 | 97 |
| | VHH3.115 | 4.09E−10 | 99 | 1.08E−09 | 100 |
| | VHH3.144 | 3.97E−10 | 98 | 1.12E−09 | 100 |
| | VHHBE4 | 2.12E−10 | 104 | 6.34E−10 | 100 |
| 36 | VHH3.36 | 1.56E−10 | 104 | 5.62E−10 | 100 |
| | VHH3.47 | 2.97E−10 | 100 | 7.29E−10 | 100 |
| 55 | VHH3.35 | 4.06E−10 | 100 | 1.06E−09 | 100 |
| | VHH3.55 | 3.22E−10 | 99 | 8.17E−10 | 100 |
| 29 | VHH3.29 | 3.89E−10 | 97 | 1.00E−09 | 97 |
| 38 | VHH3.38 | 7.69E−10 | 99 | 2.01E−09 | 99 |
| 149 | VHH3.149 | 3.34E−10 | 99 | 9.02E−10 | 98 |
| 83 | VHH3.83 | 1.62E−10 | 101 | 4.60E−10 | 74 |

The VHH families are identified/numbered in view of one of its representative VHH family members.

### Example 2. Structural Analysis Confirms the Binding Site of VHH3.38, VHH3.83 and VHH3.55 to Correspond to the VHH72 Epitope

Deep mutational scanning was performed using VHH3.38, VHH3.83 and VHH3.55 to identify that the RBD amino acid binding is comparable to the epitope of VHH72 (VHH72_h1_S56A), for which a crystal structure in complex with the related SARS-COV-1 RBD is available. We made use of a yeast-display platform developed by Starr et al. (Cell. 182, 1295-1310.e20; 2020), consisting of 2 independently generated libraries of Saccharomyces cerevisiae cells, each expressing a single RBD variant labeled with a unique barcode and a myc-tag (Greaney et al., Cell Host and Microbe, 29-1, p 44-57; 2021). The 2 libraries of RBD variants were generated by PCR-based mutagenesis to generate a comprehensive collection of RBD variants in which each position has been substituted to all other amino acids. The RBD variants contain on average 2.7 amino acid substitutions. To retain only functional RBD variants the yeast RBD-display libraries were presorted by FACS based on their ability to bind recombinant ACE2 (data not shown; reference to the examples of PCT/EP2021/052885). To identify yeast cells that express an RBD variant with reduced affinity for the tested VHHs in a sensitive manner we defined for each VHH a concertation at which binding was just below saturation. For each of the tested VHHs this concentration was first determined by staining yeast cells expressing wild type SARS-COV-2 RBD with a dilution series of VHHs. Using this approach, we selected 400 ng/ml for VHH72_h1_S56A (VHH72) and 10 ng/ml for VHH3.38, VHH3.55 and VHH3.83. To identify yeast cells expressing a RBD variant with reduced affinity for the tested VHH, the presorted library was stained with the VHH and anti-myc-tag antibody. RBD expressing cells that displayed low VHH staining were sorted, grown and used for sequencing of their respective barcodes. To identify the RBD amino acids that are significantly involved in VHH binding, the substitutions that are enriched in the sorted population were determined as described by Greane et al. (Cell Host and Microbe, 29-1, p 44-57; 2021).

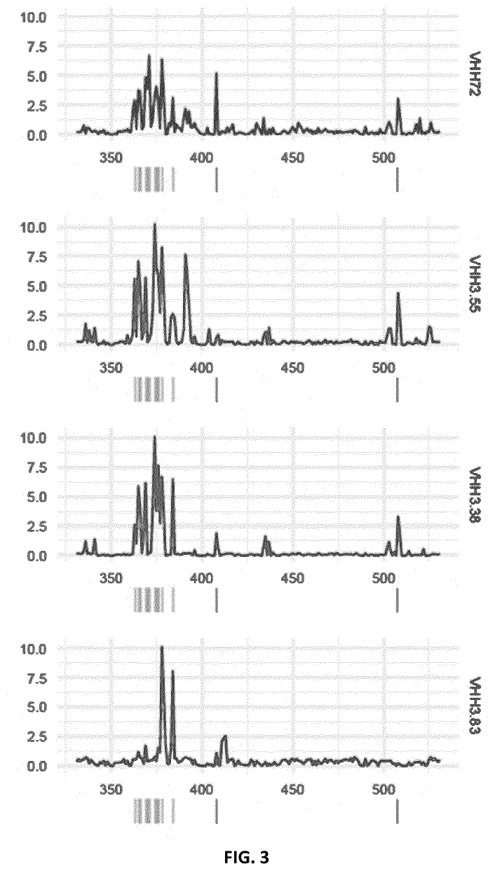

FIG. 3 shows for each tested VHH the overall profile of positions in the RBD for which substitutions result in reduced VHH binding. It is clear that the profiles for VHH3.38, VHH3.55 and VHH3.83 largely overlap with that of VHH72_h1_S56A. Escape profile analysis identified A363, Y365, S366 Y369, N370, S371, F374, S375, T379, K378, P384, and Y508 as amino acid positions that are involved (based on the average of the two libraries) in binding of VHH72_h1_S56A. Except from the 3 first positions all fall within the footprint of VHH72 on RBD as defined by modeling based on the crystal structure of VHH72 in complex with the SARS-COV-1 RBD (Wrapp, et al. 2020 Cell 181, 1004-1015.e15; Wrapp, et al. 2020; Science, 367, 1260-1263). Positions, A363, Y365 and S366 are located outside the VHH72 footprint. Inspection of the SARS-COV-2 RBD structure revealed that these are adjacent to the VHH72 epitope and that the side chains of the respective amino acids are mainly oriented inwards in the RBD. Hence, the reduction in VHH72 binding by substitutions on this position most likely results from an allosteric impact.

Only two amino acid positions (K378 and P384) of the RBD were identified in the scan for VHH3.83. Importantly, these two positions were also identified for the other tested VHHs including VHH72_h1_S56A and they are located within the VHH72 epitope. The importance of the RBD K378 residue for the binding of VHH3.83 is in line with the observation that binding of this VHH to mammalian cells expressing the SARS-COV-2 RBD K378N mutant is considerably impaired as compared to binding to wild type SARS-COV-2 RBD (data not shown; reference to the examples of PCT/EP2021/052885).

In addition to the structural analysis of VHH3.83, further VHHs of the same VHH family, as defined elsewhere herein, were identified, including SEQ ID NOs: 95-98. These VHHs that related to VHH3.83 were isolated after additional booster immunizations using the SARS-COV-2 spike protein (once) and the RBD domain (twice). The obtained VHH immune library was panned using SARS-COV-2 RBD-SD1. Among the 242 clones that were demonstrated to able to bind the SARS-COV-2 RBD-SD1 in periplasmic extracts (PE) ELISA, several VHHs had CDR3 and CDR2 amino acid sequences that are identical or highly related to those of VHH3.83. Said VHHs are thus considered to bind the same epitope, since the CDR3 is identical and their functional features are alike. Hence these are also considered ‘VHH72 epitope binders’ or binding agents for binding site 1, as described herein.

Finally, to obtain a view on the VHH3.38 binding mode and binding epitope on the SARS-COV2 spike protein (SC2), we determined the 3D cryoEM structure of SC2 in complex with the nanobody, as shown in PCT/EP2021/052885, with an electron potential map of 4.2 Å revealing density for three copies of the SC2 protomer, wherein for each of the protomers, the receptor binding domain (RBD (residues 334 to 527) was found in an upright position, in a similar conformation to that seen in the 1-RBD up conformation such as reported in PDB 6zgg.

### Example 3. Potent Neutralizing Agents of a Further VHH Family Specifically Binds the SARS-COV-2 and SARS-COV-1 Spike Proteins in a Non-Competing Mode with VHH72

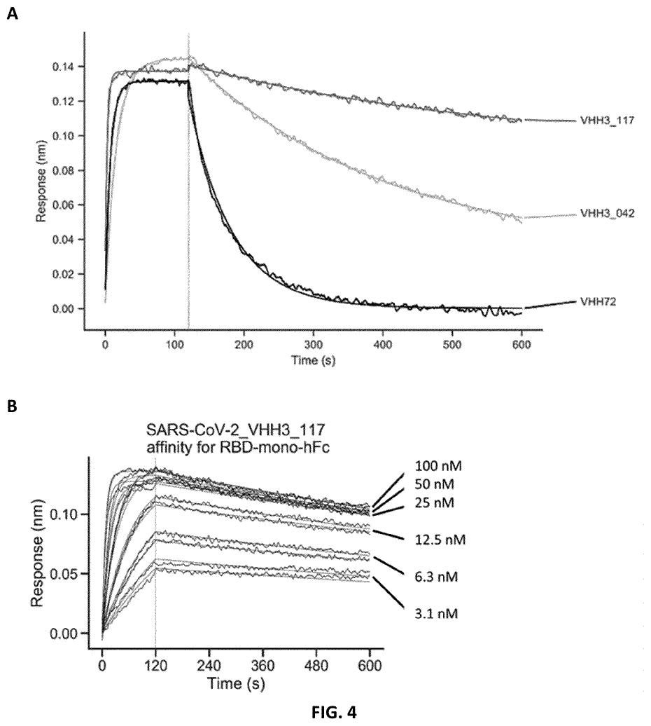

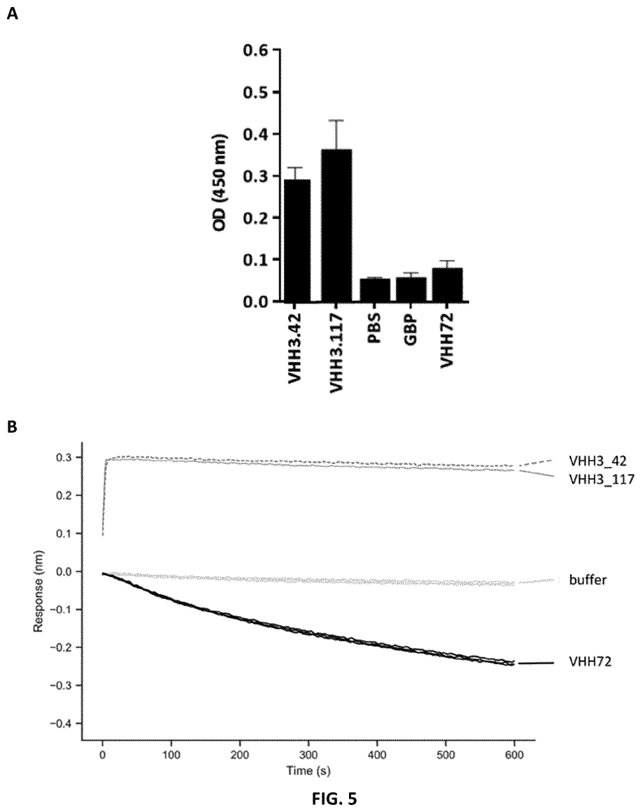

To obtain further SARS-Cov-1 and SARS-COV-2 cross reactive VHHs, a llama previously immunized with recombinant prefusion stabilized SARS-COV-1 and MERS spike protein was additionally immunized 3 times with recombinant SARS-COV-2 spike protein stabilized in its prefusion conformation (Wrapp et al. 2020, Cell 181:1436-1441). An immune VHH-displaying phagemid library was constructed, and SARS-CoV-2 spike-specific VHHs were selected using different panning strategies yielding among others a VHH family, herein called the VHH3.117 family, comprising 5 VHH family members (VHH3.117, 3.42, 3.92, 3.94, 3.180) (as described in Saelens et al. EP 21166835.5 and PCT/EP2022/052919). The binding of purified VHH3.42, VHH3.92 and VHH3.117 to the RBD of SARS-COV-2 was tested by biolayer interferometry (BLI) in which monovalent SARS-COV-2 RBD-human Fc was immobilized at 30 nM on an anti-human Fc biosensors (AMC ForteBio). This revealed that VHH3.42 and VHH3.117 bound RBD with a considerable slower off rate than VHH72 (FIG. 4A, each VHH at 200 nM). For a 200 to 3.13 nM 2-fold dilution series of VHH3.117, the binding kinetics were determined using the same BLI setup. FIG. 5B illustrates that VHH3.117 binds monomeric RBD with a K_{D} of 4.45 10⁻¹⁰ M.

To test if VHH3.42 and VHH3.117 compete with VHH72 for binding to RBD, monomeric RBD (RBD-SD1-Avi (biotinylated Avi-tag) was captured on ELISA plates coated with VHH72-S56A-Fc (D72-23=humVHH_S56A/LALAPG-Fc; Schepens et al. 2021, BioRxiv doi.org/10.1101/2021.03.08.433449); this is a VHH72-human IgG1 Fc fusion in which VHH72 has a S56A substitution in CDR2 which increases its affinity for SARS-COV-1 and -2 RBD) (FIG. 5A). VHH72 and several VHHs for which the PEs did display competition with VHH72 for the binding to the RBD were included as controls. In contrast to VHH72 and the control VHHs (not shown), VHH3.42 and VHH3.117 were able to bind monomeric RBD immobilized by VHH72-S56A-Fc (FIG. 5A). A similar competition experiment was performed by BLI in which VHH72-S56A-Fc was immobilized on anti-human Fc biosensors (AHC, ForteBio) and pretreated with RBD-muFc to allow binding of the latter to the immobilized VHH72-S56A-Fc. This biosensor was subsequently applied to a solution containing 1 μM of either VHH72-S56A-Fc, VHH3.42, VHH3.117 or only buffer. As expected, applying the biosensor probed with VHH72-huFc/RBD-muFc into a VHH72 containing solution reduced the BLI response signal, indicating the release of RBD-Fc from the biosensor. This confirms that VHH72 can compete with (displace) VHH72-S56A-Fc for the binding of RBD. In sharp contrast to this, applying a VHH72-huFc/RBD-muFc probed biosensor into a solution containing either VHH3.42 or VHH3.117 resulted in a clear enhancement of the BLI response signal (FIG. 5B). This illustrates that VHH3.117 and VHH3.42 can bind the RBD at a site that is distant from the VHH72 epitope.

Purified VHH3.42, VHH3.117 and VHH3.92 were tested in neutralization assays using pseudotyped VSV-delG containing the spike protein of SARS-COV-2 or SARS-COV-1. Table 2 illustrates that VHH3.42, VHH3.117 and VHH3.92 could neutralize pseudotyped VSV-delG containing the spike protein of SARS-CoV-2, and this about 6 times more efficiently than VHH72_h1_S56A. We also tested if VHH3.42 and VHH3.117 could also neutralize SARS-COV-1. Table 2 illustrates that both VHH3.42 and VHH3.117 could potently neutralize VSV-delG pseudotyped with SARS-COV-1 spike. For both SARS-COV-1 and SARS-COV-2 the neutralizing activity of VHH3.117 was somewhat higher than that of VHH3.42.

TABLE 2
The IC50 values of independent neutralization
assays using pseudotyped VSV-delG containing the spike
protein of SARS-COV-2 or SARS-COV-1. (NT = not tested)

| | IC₅₀ Mean VSV-dG- | IC₅₀ Mean VSV-dG- |
|---|---|---|
| | spike SARS-COV-2 | spike SARS-COV-1 |
| VHH3.42 | 0.80 ± 0.76 ug/ml (n = 5) | 0.19 ug/ml (n = 1) |
| VHH3.117 | 0.20 ± 0.15 ug/ml (n = 6) | 0.016 ug/ml (n = 1) |
| VHH3.92 | 0.21 ± 0.13 ug/ml (n = 2) | NT |
| VHH72_h1_S56A | 1.14 ± 0.67 ug/ml (n = 5) | 0.013 ug/ml (n = 1) |

### Example 4. VHH3.42, VHH3.117 and VH3.92 do not Prevent Binding of RBD to its Receptor, ACE2

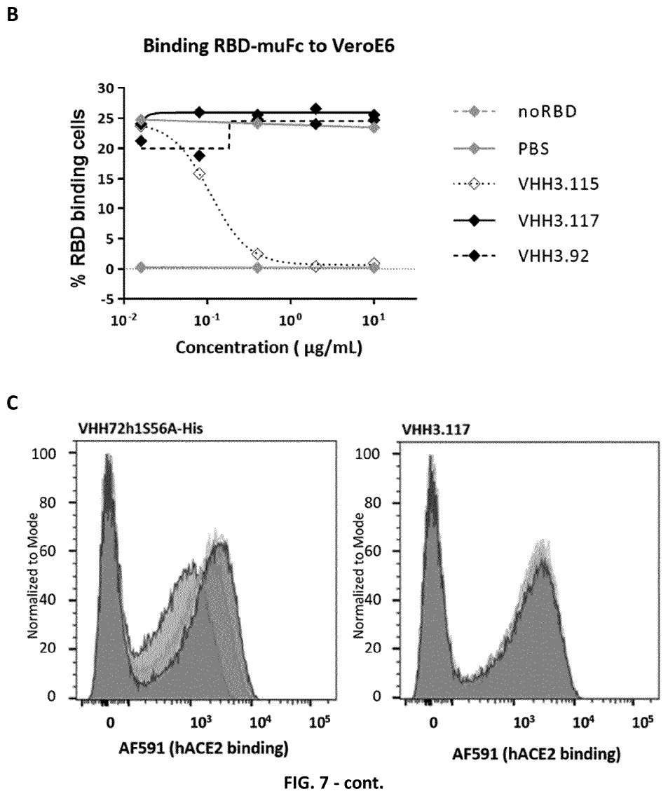

Most reported monoclonal antibodies and VHHs neutralize by preventing the binding of RBD to its receptor ACE2. Although VHH72 binds the RBD outside its receptor-binding motif (RBM) it prevents RBD from binding to ACE2 by steric hindrance (Wrapp et al. 2020, Cell 181:1436-1441). To investigate if the neutralizing VHHs identified herein are able to inhibit binding of RBD to ACE2, we investigated the impact of these VHHs on the interaction of recombinant RBD with recombinant ACE2 proteins by AlphaLISA. Serial dilutions of VHHs (final concentration ranging between 90 nM-0.04 nM) were made in assay buffer (PBS containing 0.5% BSA and 0.05% Tween-20), and mixed with SARS-COV-2 RBD that was biotinylated through an Avi-tag (AcroBiosystems, Cat nr. SPD-C82E9) (final concentration 1 nM) in white low binding 384-well microtiter plates (F-bottom, Greiner Cat nr 781904). Recombinant human ACE-2-Fc (final concentration 0.2 nM) was added to the mixture. After 1 hour incubation at room temperature, donor and acceptor beads were added to a final concentration of 20 μg/mL for each in a final volume of 0.025 mL. RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C). The mixed beads were incubated for an additional 1 hour at room temperature in the dark. Interaction between beads was assessed after illumination at 680 nm and reading at 615 nm on an Ensight instrument. In contrast to VHH72 and the related VHH3.115, neither of the herein identified VHH3.42, VHH3.117 and VHH3.92 could interfere with the RBD/ACE2 interaction even at doses well above their respective neutralization IC50 (54.8 nM, 13.7 nM and 13.55 nM) (FIG. 7).

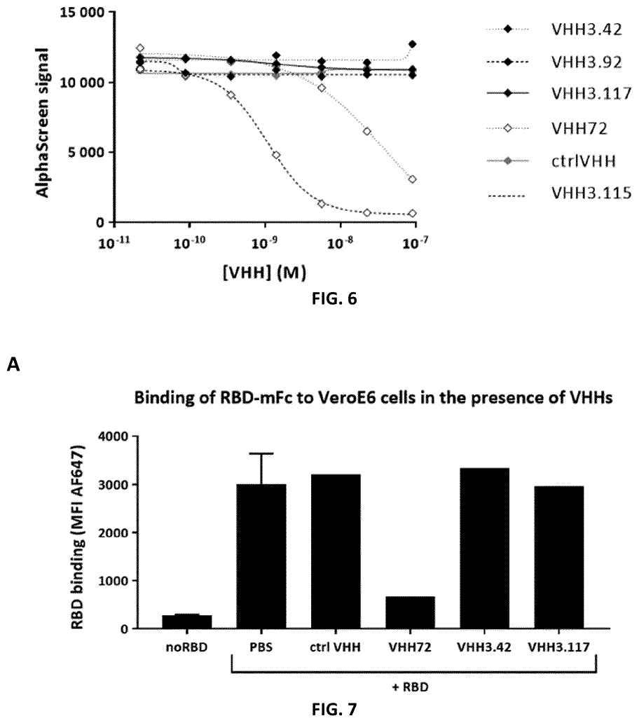

To investigate if the herein identified VHHs are also unable to inhibit binding of RBD to ACE2 expressed at a cell surface, we determined binding of bivalent SARS-COV-2 RBD, fused to a mouse Fc, to Vero cells (FIG. 6). FIGS. 7A and 6B illustrate that VHH3.42, VHH3.117 and VHH3.92 could not prevent the interaction of bivalent SARS-COV-2 RBD with VeroE6 cells, even at concentrations well above their respective neutralization IC50 (Table 2). This indicates that these VHHs neutralize SARS-COV infections via an alternative mechanism that does not involve prevention of RBD mediated viral attachment to target cells.

Next, we tested if VHHs of the VHH3.42 family would also fail to interfere with the binding of recombinant ACE2 to cell-surface expressed RBD. Therefore, we investigated if VHH72 or VHH3.117 can prevent the binding of recombinant ACE2 fused to a mouse Fc to RBD expressed at the surface of yeast cells (FIG. 7C). As expected, VHH72 (VHH72_h1_S56A) could inhibit the binding of recombinant ACE2-Fc to yeast cells that express SARS-COV-2 RBD at their cell surface. In contrast, VHH3.117 could not do so.

Taken together these data consistently demonstrate that herein identified VHHs cannot prevent binding of RBD to ACE2, i.e. the canonical sarbecovirus (such as SARS-COV-1 and -2) receptor that is expressed at the surface of target cells. This indicates that these VHHs neutralize sarbecovirus infections via an alternative mechanism.

### Example 5. VHH3.117-Family Members Bind a Conserved Epitope in the Sarbecovirus RBD that is Distant from that of VHH72

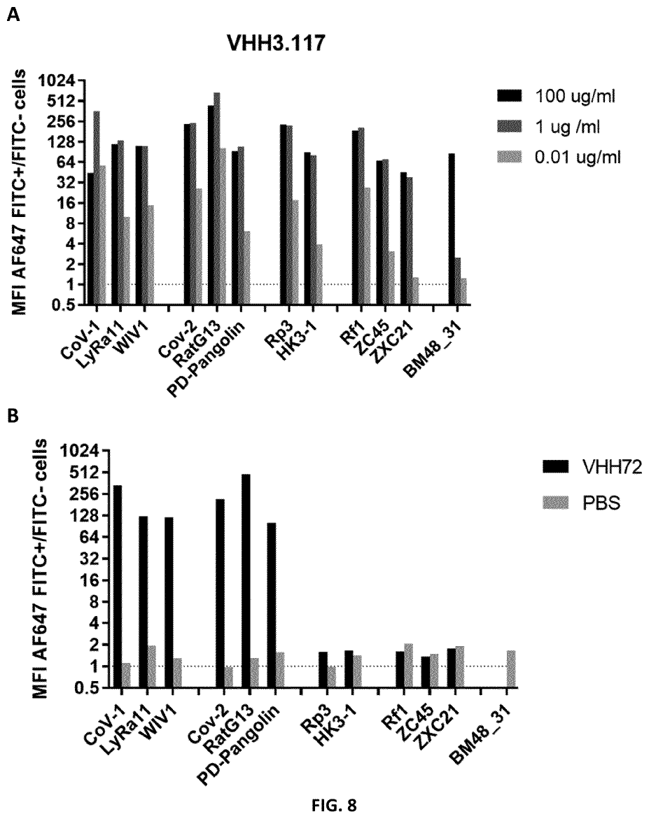

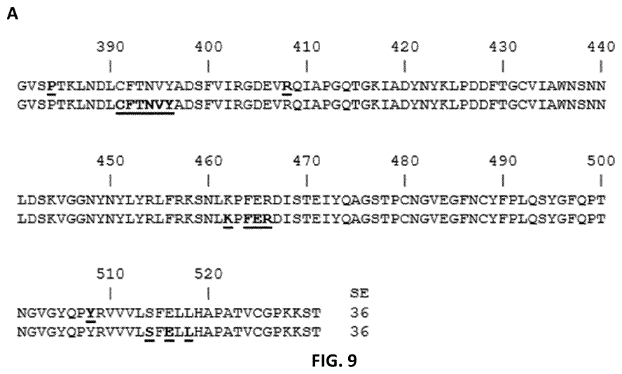

The observation that the VHH3.117 family does not compete with VHH72 or ACE2 for RBD binding, illustrates that these VHHs bind to an epitope that is distant from VHH72 and from the RBM (receptor binding motif (sub)domain in the RBD). To further delineate the epitope of the VHH3.117 family and to define their potential for cross-reacting with other sarbecoviral RBDs, we investigated their binding to the RBD of various sarbecoviruses. To this end, binding of these VHHs to yeast cells expressing the RBD of representative clade 1.A (WIV1), clade1.B (GD-pangolin), clade 2 (HKU3 and ZCX21) and clade 3 (BM48-31) sarbecoviruses was tested by flow cytometry. All tested VHHs (at 10 μg/ml), except for the GBP (GFP binding protein) control VHH, bind yeast cells expressing the RBD of clade 1.A (WIV1) and clade1.B (GD-pangolin) at their surface (data not shown; EP21166835.5 and PCT/EP2022/052919). In addition, VHH3.117, VHH3.42 and VHH3.92 are able to bind to the RBD of HKU3 and ZXC21, representing the two clade 2 branches. Moreover, VHH3.42, VHH3.92 and to a lesser extent VHH3.117 could also bind to the RBD of the clade 3 BM48-31 sarbecovirus (data not shown; reference herein to examples in EP21166835.5 and PCT/EP2022/052919). In a separate experiment, the binding of VHH3.117 to a broader range of clade 1, 2 and 3 sarbecoviruses was tested. FIG. 8A illustrates that VHH3.117 can bind to all tested RBD variants, and is binding to more RBD variants compared to VHH72 (FIG. 8B). These observations are in line with the hypothesis that VHH3.117 targets an RBD region that is highly conserved among the tested RBD variants.

To determine the binding site of the VHH3.117 family binders on the RBD we performed deep mutational scanning. VHH72 (VHH72_h1_S56A), for which a crystal structure in complex with the related SARS-COV-1 RBD is available, was included as a reference (Wrapp et al. 2020, Cell 181:1436-1441; Schepens et al., doi.org/10.1101/2021.03.08.433449), and performed as described in the present application in Example 2.

For each of the tested VHHs this concentration was first determined by staining yeast cells expressing wild type SARS-COV-2 RBD with a dilution series of VHHs. Using this approach, we selected 400 ng/ml for VHH72_h1_S56A (VHH72) and 100 ng/ml for VHH3.117.

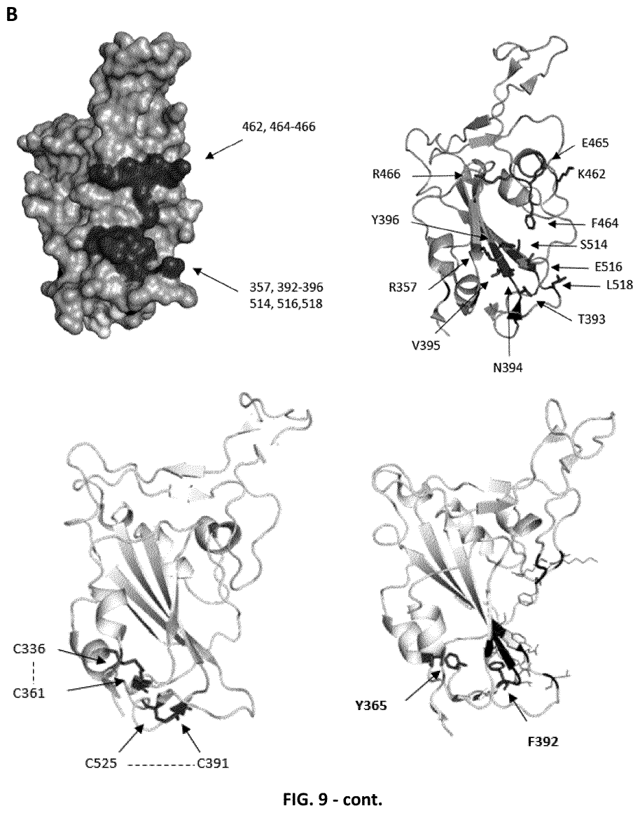

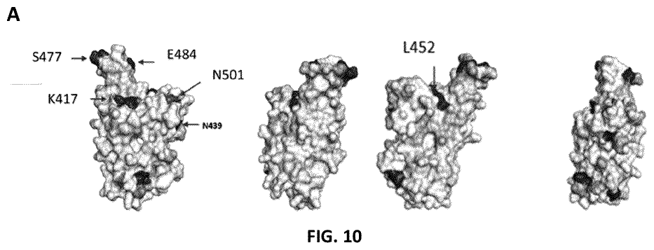

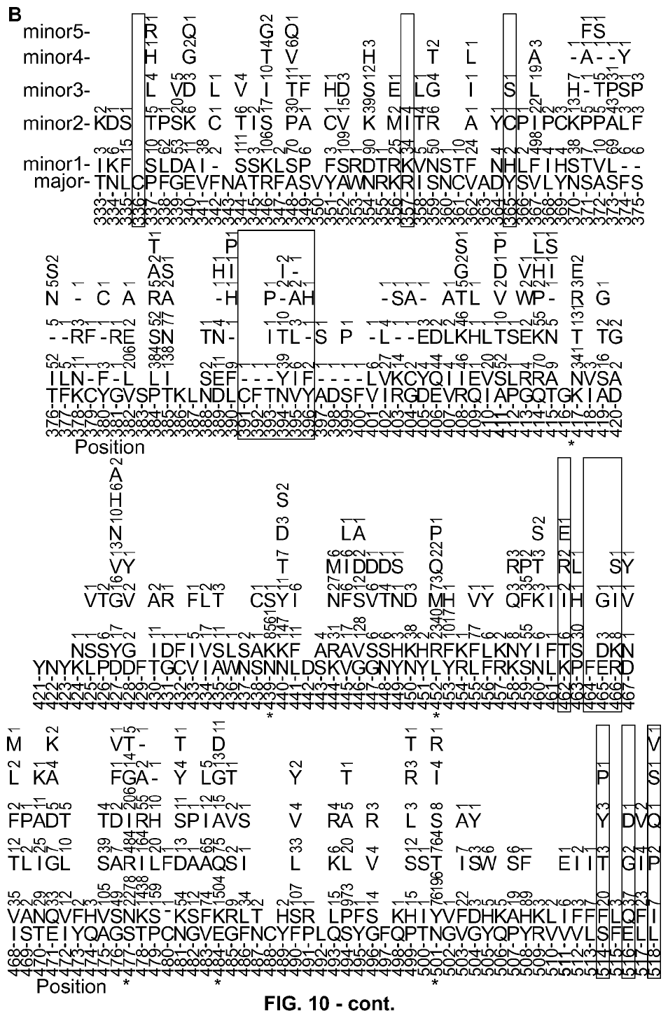

FIGS. 9A and 10A shows for the two tested VHH the overall profile of positions in the RBD for which substitutions result in reduced VHH binding. It is clear that VHH3.117 and VHH72_h1_S56A have very distinct RBD binding profiles. Escape profile analysis as established by Greaney et al. 2021 (supra), identified A363, Y365, S366 Y369, N370, S371, F374, S375, T376, K378, P384, and Y508 as amino acid positions that are involved (based on the average of the two libraries) in binding of VHH72_h1_S56A. For VHH3.117, the escape profile analysis identified C336, R357, Y365, C391, F392, T393, N394, V395, Y396, K462, F464, E465, R466, S514, E516 and L518 as important for RBD binding (FIGS. 9A and 9B). Except for C336, Y365, C391 and F392 all these amino acids cluster around a cleft at the side to the RBD that represents the likely VHH3.117 binding site based on the above described experiments. This binding site is also in agreement with the general preference of VHHs to bind clefts rather than protruding protein surfaces. C336 and C391 form disulfide bridges with respectively C361 and C525 that are likely very important for the overall stability of the RBD, explaining why these residues were identified by the deep mutational scanning (FIG. 9B). Y365 and F392 locate near the likely VHH3.117 binding surface and are oriented towards the inside of the RBD core (FIG. 9B). Hence, mutations at those positions can have an allosteric impact on the binding of VHH3.117. Deep mutational scanning revealed that Y365 is also important for VHH72 binding. Y365 is located in the RBD core at a site that is opposite of the VHH3.117 binding region. Likewise, Y365 does not locate at the RBD surface that is recognized by VHH72 but is oriented toward the inner RBD core between the VHH3.117 and VHH72 binding regions. This indicates that Y365 is important for the overall conformation of the RBD core. Importantly, the identified VHH3.117 binding site is in agreement with our findings that VHH3.117 does not compete with ACE2, and VHH72 for the binding of RBD, in agreement with its ability to bind to the RBD of clade 1, 2 and 3 sarbecoviruses and in agreement with its SARS-COV-1 and -2 cross-neutralizing activity. Analysis of the amino acid variations among circulating SARS-COV-2 viruses for which the genome sequence was submitted to GiSAID on the surface of the RBD revealed that the VHH3.117 binding region as identified by deep mutational scanning is highly conserved as illustrated by the projection of those variations on the RBD surface (FIG. 10A).

Binding of herein identified VHHs to the RBD does not interfere with binding of RBD to ACE2 at the surface of target cells. Consequently, these VHHs prevent infection via an alternative mechanism, for example by locking the SARS-COV-2 spike in its inactive closed conformation as has been described for S309 and mNb6-tri (Pinto et al. 2020, Nature 583:290-295; Schoof et al. 2020, Science 370: 1473-1479). To get insight in the mechanism by which VHH3.117 related VHHs can neutralize SARS-COV-1 we displayed the VHH3.117 binding site on a Spike timer with 1 RBD in up-conformation. This reveals that the VHH3.117 site is almost completely occluded on the RBDs that are in the down-conformation. Moreover, on RBDs in up-conformation the VHH3.117 binding site is largely shielded by the NTD of a second spike protomer (FIG. 10B). This demonstrates that VHH3.117 and related VHHs neutralize via mechanism that does not involve locking the RBD in its down-conformation but rather by interfering with the overall spike conformation and/or function.

The VHH3.117 epitope comprises one or more of the SARS-COV-2 RBD amino acids Arg357, Thr393, Asn394, Val395, Tyr396, Lys462, Phe464, Glu465, Arg466, Ser514, Glu516 and/or Leu518 (with Cys336, Tyr 365, Cys391, Phe392 being important to keep the RBD in a conformation recognized by VHH-117). Overall, VHH3.117 does not bind to RBD amino acids known to be prone to variation in newly emerging SARS-COV-2 strains (South African and Brazilian strains: variations in Lys417, Glu484, Asn501; Californian strain: variation in Leu452; British strain: variation in Glu484).

### Example 6. VHH3.117 and VHH72 Co-Operate in SARS-COV-2 Neutralization

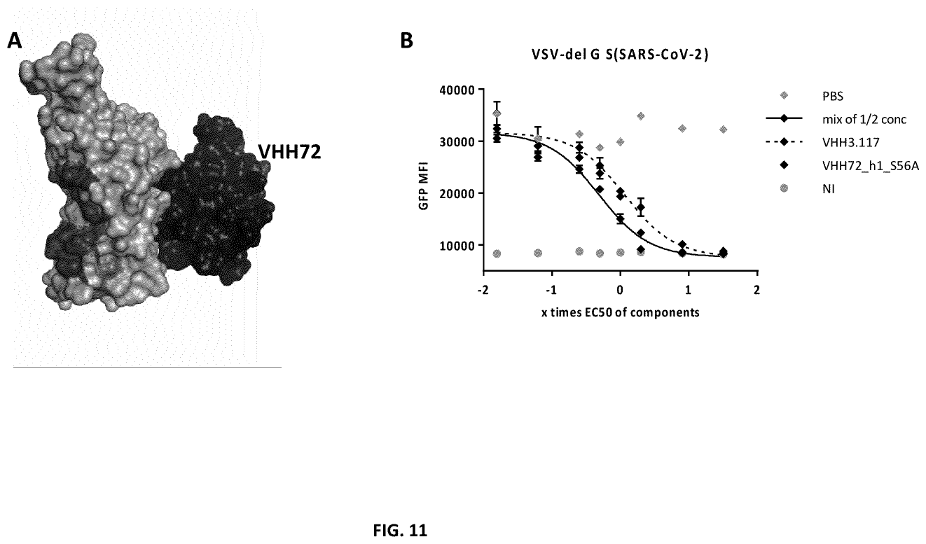

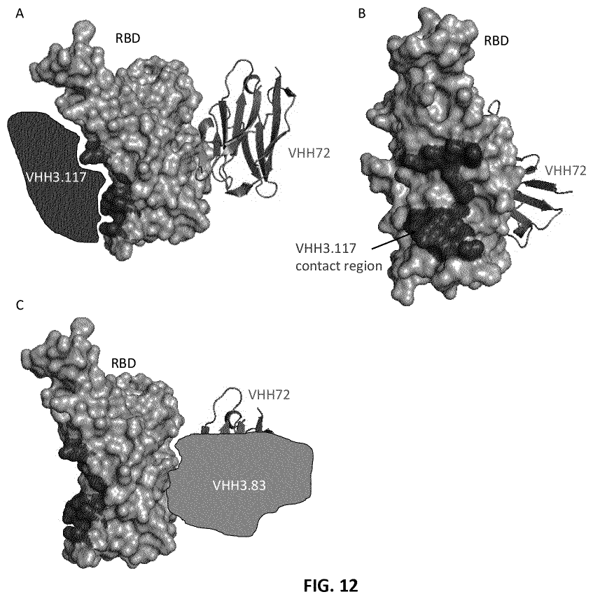

From our previous observations that VHH72 and VHH3.117 do not compete for RBD binding because they target distant binding sites (FIG. 11A), we anticipated that these VHHs would most likely not interfere with their reciprocal neutralization activity. Therefore, we tested the neutralizing activity of a cocktail of VHH72_h1-S56A and VHH3.117. To test this we performed VSV-DG spike (SARS-COV-2V) neutralization assays using serial dilutions of x times the EC50 (for VSV-DG spike SARS-COV-2 neutralization) concentrations of VHH72 and VHH3.177 and 1:1 mixtures of VHH72 and VHH3.117 at half their corresponding EC50 concentrations. FIG. 11B shows that the cocktail of VHH72 and VHH3.117 has higher neutralization activity then the corresponding individual VHHs.

### Example 7. Design and Generation of Different Constructs of Bispecific-Antibodies Based on RBD-Specific VHHs

FIG. 12 shows a model visualizing the binding positions of ‘VHH72-epitope’ (or ‘binding site 1’ as defined herein) binders and of ‘VHH3.117-epitope’ (or ‘binding site 2’ as defined herein) binders on the RBD of the Spike protein of SARS-COV-2, indicating that antibodies targeting both binding sites should be made by fusion of binding agents for instance constituting VHH-building blocks fused by a suitable linker, or by another fusion protein, such as an Fc fusion. FIG. 13 provides for several non-limiting examples of proposed fusion constructs for the design of bispecific antibody compositions, as to target the VHH72- and the VHH3.117-epitopes simultaneously with one antibody composition. Starting from a VHH72-epitope binder (or a humanized variant thereof), and a VHH3.117-epitope binder (or humanized variant thereof), different bispecific or tetravalent fusion constructs are designed and produced, and compared for their functionality with for instance homobivalent or monovalent constructs of the same building blocks. For instance, starting from VHH72 itself, or a VHH72-family member, or another VHH family identified herein as VHH72-epitope binding, such as VHH3.83, or a family member thereof, as ‘binding site 1’ building blocks, or mutant or humanized variants thereof, in combination with VHH3.117 itself, or a VHH3.117 family member, or another VHH family identified herein as VHH3.117-epitope binder, such as VHH3.89, or a family member thereof, as identified herein (Example 9), as ‘binding site 2’ building block, or mutant or humanized variants thereof, a number of fusions can be proposed, in different manners, as bispecifics coupled by a linker (or directly), or as fusions to an Fc domain, or as bispecifics fused by a linker fused to an Fc tail, or as a fusion made by another functional moiety, such as another VHH building block. Some non-limiting examples of specific fusions are provided in Table 3. Alternative fusion types or alternatives to binding site 1 and binding site 2 building blocks may be envisaged as well, as also any combination thereof.

The antibody compositions are expressed in Pichia pastoris and/or in CHO cells, or any alternative suitable production host, followed by purification and biochemical and biophysical characterization, as described herein and/or as known by the skilled person. The RBD binding characteristics, such as affinity, competition profiled as compared to other RBD binders and as compared to the human receptor binding the RBD, such as ACE2, as well as the potency of each composition or binding agent is analyzed as known to the skilled person.

TABLE 3
Antibody constructs for recombinant production.

| | RBD | | SEQ ID NO: |
|---|---|---|---|
| | binding | | (protein |
| VHH type | site | Construct Name | insert) |
| monovalent | 1 (=VHH72 | px-M001_GS-VHH72-h1-E1D-S56A_His8 | 5 |
| | epitope) | | |
| monovalent | 1 | px-M005_GS-VHH72-h1-E1D-S56A_His8 (noNtermGS) | 5 |
| monovalent | 1 | pX-M006_GS-VHH3-83-hc_His8 | 7 |
| monovalent | 1 | px-M007_GS-VHH3-83-hc-N85E_His8 | 8 |
| monovalent | 1 | px-M002_GS-VHH72-h1-E1D-R27L-E31D-Y32I-S56G-L97A_His8 | 90 |
| bivalent | 1 | pX-B001_GS-VHH72-h1-E1D-S56A_(G₄S)₆_VHH72-h1-E1D- | 73 |
| | | S56A_His8 | |
| bivalent | 1 | px-B002_GS-VHH72-h1-E1D-S56A_(G₄S)_VHH72-h1-E1D- | 74 |
| | | S56A_His8 | |
| bivalent | 1 | pX-B003_GS-VHH72-h1-E1D-S56A_(G₄S)₄_VHH72-h1-E1D- | 75 |
| | | S56A_His8 | |
| bispecific | 2 | px-B004_GS-VHH3-117-hc_(G₄S)₆_VHH72-h1-E1D-S56A_His8 | 76 |
| | (=VHH3.117 | | |
| | epitope)+1 | | |
| bispecific | 2 + 1 | pX-B005_GS-VHH3-117-hc_(G₄S)_VHH72-h1-E1D-S56A _His8 | 77 |
| bispecific | 2 + 1 | pX-B006_GS-VHH3-117-hc_(G₄S)₄_VHH72-h1-E1D-S56A_His8 | 78 |
| bispecific | 2 + 1 | pX-B007_GS-VHH3-117-hc_(G₄S)₆_VHH3-83-hc_His8 | 79 |
| bispecific | 2 + 1 | pX-B008_GS-VHH3-117-hc_(G₄S)₆_VHH3-83-hc-N85E_His8 | 80 |
| bispecific | 2 + 1 | px-B009_GS-VHH3-117-hc_(G₄S)_VHH3-83-hc_His8 | 81 |
| bispecific | 2 + 1 | pX-B010_GS-VHH3-117-hc_(G₄S)₄_VHH3-83-hc_His8 | 82 |
| bispecific | 2 + 1 | pX-B011_GS-VHH3-117-hc_(G₄S)_VHH3-83-hc-N85E_His8 | 83 |
| bispecific | 2 + 1 | pX-B012_GS-VHH3-117-hc_(G₄S)₄_VHH3-83-hc-N85E_His8 | 84 |
| bispecific | 2 + 1 | px-B014_GS-VHH3_117-hc_(G4S)6_VHH72-h1-E1D-R27L-E31D- | 91 |
| | | Y32I-S56G-L97A_His8 | |
| bispecific | 2 + 1 | px-B017_GS-VHH3_117-hc_(G4S)_VHH72-h1-E1D-R27L-E31D- | 92 |
| | | Y32I-S56G-L97A_His8 | |
| bispecific | 2 + 1 | pX-B018_GS-VHH3_117-hc_(G4S)4_VHH72-h1-E1D-R27L-E31D- | 93 |
| | | Y32I-S56G-L97A_His8 | |
| bivalent-Fc | 1 | VHH72_h1_E1D_R27L_E31D_Y32I_S56G_L97A-10xGS- | 88 |
| | | hlgG1_EPKSCdel_LALA_K447del | |
| bivalent-Fc | 1 | VHH72-h1-E1D-R27L-E31D-Y32I-S56G- | 89 |
| | | L97A_(GGGGS)x2_hlgGhingeEPKSCdel_hlgGFc_N297A_Gsdel | |

The previously developed ‘VHH72-h1-E1D-S56A’ variant of the originally identified VHH72 from the llama immune library has been described in Schepens et al. 2021 (Biorxiv doi: https://doi.org/10.1101/2021.03.08.433449) and in PCT/EP2021/052885.

A further variant of VHH72 is provided by ‘VHH72-h1-E1D-R27L-E31D-Y32I-S56G-L97A’ wherein 5 additional substitutions were made based on the original sequence of VHH3.115 (SEQ ID NO:12).

For the VHH3.83 nanobody, known to bind ‘binding site 1’ of the RBD as defined herein (Example 2), an N-glycosylation site is present in its original amino acid sequence at position 85, so one of the mutant variants of this building block is the substituted glycosylation site by a glutamate residue (N85E) to prevent glycosylation when expressed in eukaryotic cells.

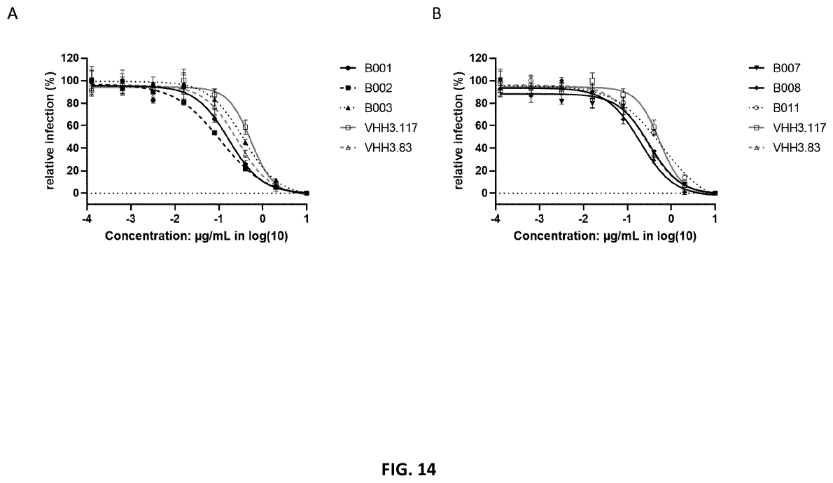

The Pichia expression of such a non-glycosylated mutant VHH3.83-N85E has been tested in a VHH83-VHH117 bispecific format, and showed not to significantly reduce the expression levels and retained potency (FIG. 14: and Table 4; ‘b7’ as compared to ‘b8’ construct; Example 8).

TABLE 4
IC50 and EC50 Values obtained from the pseudoneutralization assays as shown in FIG. 14.

| | IC50 ug/ml | EC50 nM |
|---|---|---|
| pX-B001_GS-VHH72-h1-E1D-S56A (G4S)6_VHH72-h1-E1D-S56A_His8 | 0.1691 | 5.54 |
| pX-B002_GS-VHH72-h1-E1D-S56A (G4S) VHH72-h1-E1D-S56A_His8 | 0.1081 | 3.74 |
| pX-B003_GS-VHH72-h1-E1D-S56A_(G4S)4_VHH72-h1-E1D-S56A_His8 | 0.3615 | 12.10 |
| pX-B007_GS-VHH3-117-hc_(G4S)6_VHH3-83-hc_His8 | 0.3116 | 10.20 |
| pX-B008_GS-VHH3-117-hc_(G4S)6_VHH3-83-hc-N85E_His8 | 0.1963 | 6.42 |
| pX-B011_GS-VHH3-117-hc_(G4S)_VHH3-83-hc-N85E_His8 | 0.6087 | 21.01 |
| VHH3.117 (His) | 0.5387 | 36.69 |
| VHH3.83 (HA + HIS) | 0.262 | 16.17 |

Examples of humanization variants include for instance the ‘VHH3_83 hc’ sequence which has the following substitutions as compared to the originally identified llama VHH3.83: Q1D, K83R, Q108L.

Similarly, for VHH3.117, a humanized version named ‘VHH3-117-hc’ as provided in Table 3 constitutes a variant with substitutions Q1D, Q5V, K83R, and Q108L.

Based on the constructs and analysis herein, it is clear for the skilled person to design variant bispecific constructs using a peptide linker, an Fc domain, or any combinations thereof.

### Example 8. Neutralization Potential of Bispecific VHHs for SARS-COV-2

An initial set of bivalent/bispecific antibody constructs was produced and analyzed for their neutralizing activity. A VSV-DG spike (SARS-COV-2V) neutralization assay was preformed using serial dilutions of the constructs indicated in FIG. 14: ‘pX-B1’, ‘pX-B2’ and ‘pX-B3’ constructs encoding homobivalent VHH72-h1-S56A proteins with different GS linkers, used for comparison in neutralization potential to the bispecific formats combining VHH3.83, also a VHH72 binding site binder, and VHH3.117, encoded by ‘pX-B7’, ‘pX-B8’ and ‘pX-B11’. These data demonstrate that the bispecific VHHs provide for a neutralization activity that is at least in the same order of magnitude as the homobivalent constructs, and upon further optimization of the linkers, the position of the VHHs and variant sequences reveal suitable candidates for therapeutic use in SARS-COV-2 treatment. Moreover, taking into account the conserved nature of both binding sites, which is indicative for reduced chance of escape mutants, these bispecifics are the basis for developing potent pan-specific sarbecovirus antibodies.

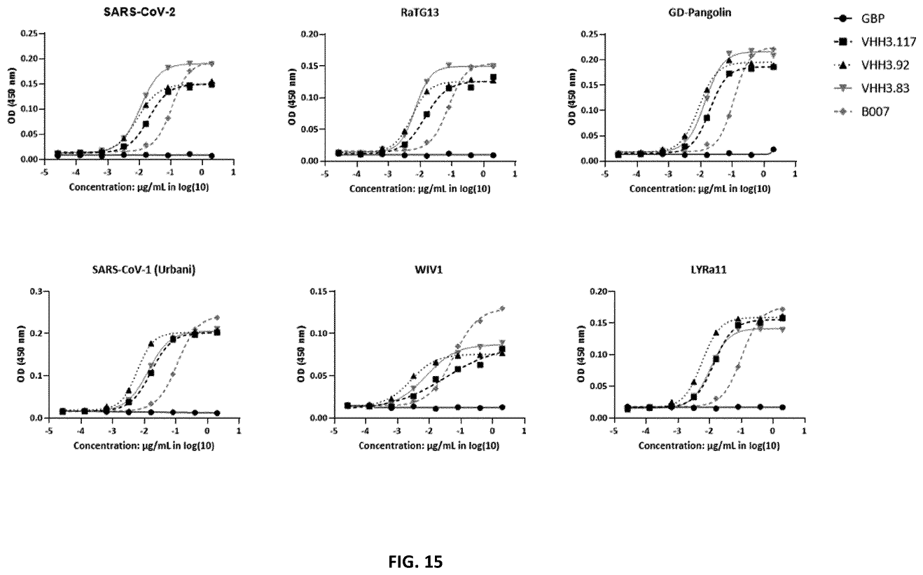

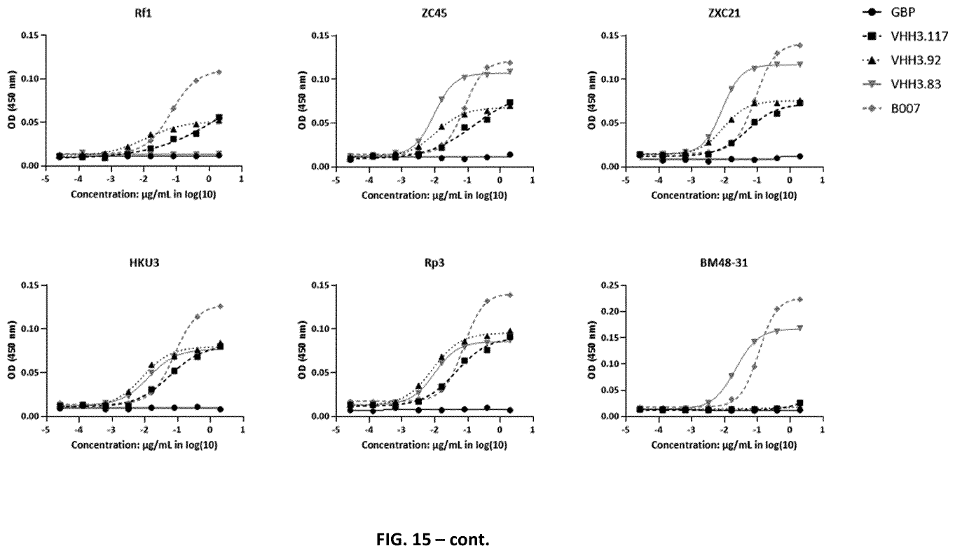

Previous flow cytometric analysis revealed that VHH3.117 can potently bind to the RBD of clade 1 and clade 2 sarbecoviruses and to the RBD of clade 3 BM48-31 sarbecovirus, although with reduced affinity (see EP21166835.5 and PCT/EP2022/052919). In contrast, the BM48-31 was efficiently recognized by VHH3.83. In addition, VHH3.83 could also potently bind to the RBD of all tested clade 1 sarbecoviruses (SARS-COV-2, PD-pangolin, RaTG3, SARS-COV-1, LYRa11) and to the RBD of all tested clade 2 sarbecoviruses (HKU3-1, Rp3, ZXC21, ZC45) except for Rf1 (data not shown). Clear binding of VHH3.83 to yeast cells expressing the RBD of Rf1 could be observed at 100 but not at 1 μg/ml (data not shown). To test if bivalent VHHs comprising VHH3.117 and VHH3.83 (e.g. B007) could potently bind to the RBD of all tested clade 1, 2 and 3 sarbecoviruses we investigated the binding of VHH3.117, VHH3.83 and B007 (encoded by pX-B7) to yeast cells expressing the respective RBD at their surface by yeast cell ELISA. FIG. 15 illustrates that B007 can potently bind to all tested RBDs including the ones that are poorly recognized by its respective monovalent VHHs (VHH3.117 and VHH3.83).

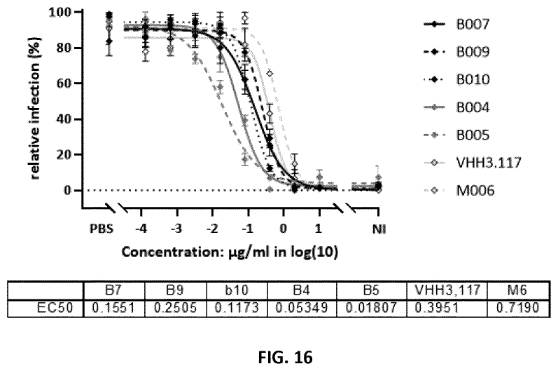

In addition, the pseudoneutralization assay was performed using bispecific constructs expressed from px-B7, pX-B9 and pX-B10 encoding VHH3.117 fused to VHH3.83, or pX-B4 and pX-B5 encoding VHH3.117-VHH72 binding bispecifics. As shown in FIG. 16, the VHH3.117-VHH72-S56A is more potent than VHH3.117-VHH3.83, although as monovalent VHH3.83 has higher affinity than VHH72-S56A. Another observation was that the GS linker length did not seem to strongly impact the potency VHH3.117-VHH72, though with the shorter 1×G4S resulting in the least favourable EC50. On the other hand, for VHH3.117-VHH3.83 the shorter 1×G4S was 3.5× more potent than 6×G4S linker.

### Example 9. Identification of the VHH3.89 Family as Binding Agents for the VHH3.117 Epitope

VHH3.89 (SEQ ID NO:85) was identified as previously reported (PCT/EP2021/052885), and several additional family members of this Nb have been revealed herein, corresponding to VHH3_183, and VHH3C_80 (respectively depicted in SEQ ID NO:86-87).

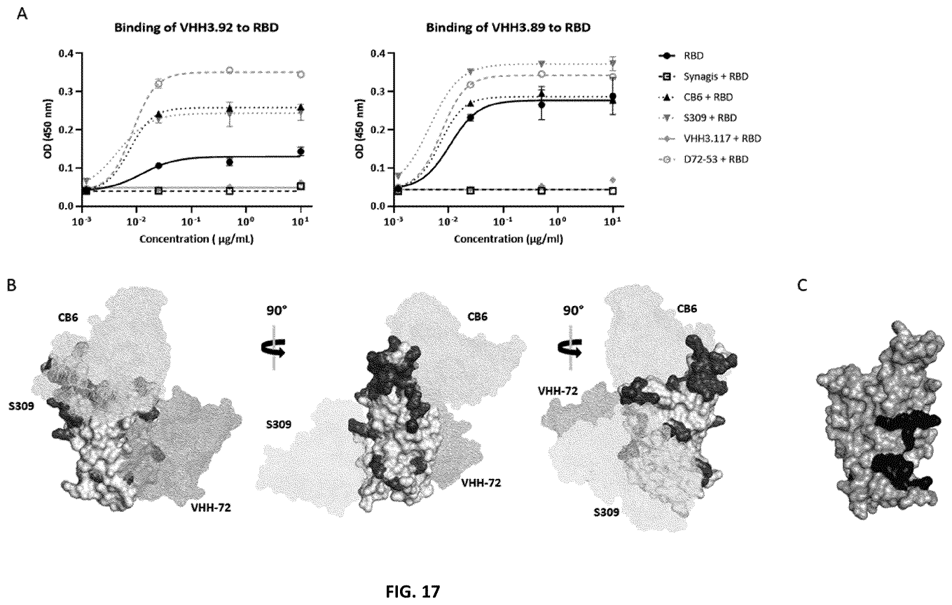

Previous analysis revealed that next to VHH3.117 also VHH3.89 does not compete with VHH72 for the binding of the SARS-COV-2 RBD (see FIG. 2). To confirm this and to further characterize the binding site of VHH3.89 binding of this VHH to monovalent RBD that was either directly coated to ELISA plates or captured by coated monoclonal antibodies S309, CB6 or by VHH3.117 or by VHH72-S56A fused to a human IgG1 Fc (D72-53=VHH72_h1_E1D_S56A-(G4S)2-hIgG1hinge_EPKSCdel-hIgG1_LALA_Kdel) was investigated (Pinto et al., Nature, 2020; Shi et al., Nature 2020). FIG. 17A demonstrates that VHH3.89 just like VHH3.92, a VHH that belongs to the family of VHH3.117, does not compete with S309, CB6 and D72-53 but does compete with VHH3.117. This demonstrates that the binding site of VHH3.89 overlaps with that of VHH3.117 and VHH3.92 (FIG. 17).

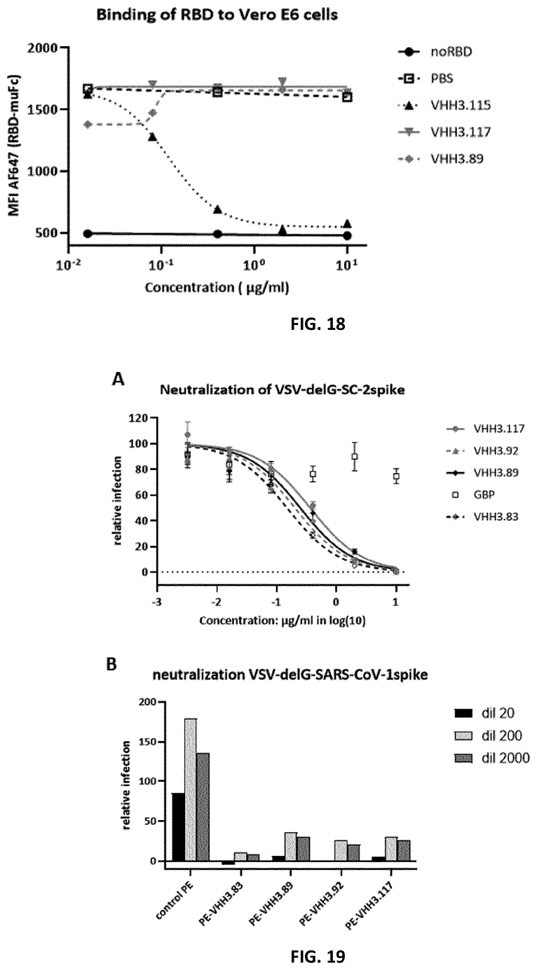

The binding site of VHH3.117 on the RBD is distant from the ACE2 binding region and consequently VHH3.117 and related VHHs fail to prevent binding of RBD to ACE2 (as previously shown in EP21166835.5 and PCT/EP2022/052919). Using AlphaLISA we previously demonstrated that also VHH3.89 does not interfere with the binding of the RBD to recombinant ACE2 in solution (PCT/EP2021/052885, FIG. 47). To confirm that VHH3.89 can also not prevent the binding of SARS-COV-2 RBD to the human receptor on the surface of target cells, we tested the binding of RBD-muFc that was pre-incubated with VHH3.89 to Vero E6 target cells. VHH3.117 and VHH3.115, which is related to VHH72 and known to prevent RBD from binding ACE2, were used as controls. FIG. 18 shows that just like VHH3.117, VHH3.89 cannot prevent the binding of RBD to ACE2 expressing Vero E6 cells at concentrations above its EC50 for neutralization of VSV-delG pseudotyped with the SARS-COV-2 spikes (see below and FIG. 19).

To test if, similar to VHH3.117, VHH3.89 can neutralize SARS-COV-2 without being able to block binding of RBD to ACE2, we investigated if VHH3.89 can neutralize SARS-COV-2 spike pseudotyped VSV-delG. A GFP targeting VHH (GBP) was used as negative control, VHH3.117 and VHH3.92 were used as references and VHH3.83 that bind to the VHH72 epitope and does interfere with RBD binding to ACE2 was used as positive control (PCT/EP2021/052885). FIG. 19A illustrates that VHH3.89 neutralizes VSV-del G pseudotyped with SARS-COV-2 spikes with an EC50 that is comparable to that of VHH3.117 and VHH3.92. In addition, PE extracts containing VHH3.89, VHH3.83, VHH3.117 or VHH3.92 were also able to neutralize SARS-COV-1 spike pseudotyped VSV-delG. Taking into account the variation between the RBDs of SARS-COV-2 and -1 this cross-neutralizing activity underscores that VHH3.117 and VHH3.92 bind highly similar epitopes (FIGS. 17 B and C).

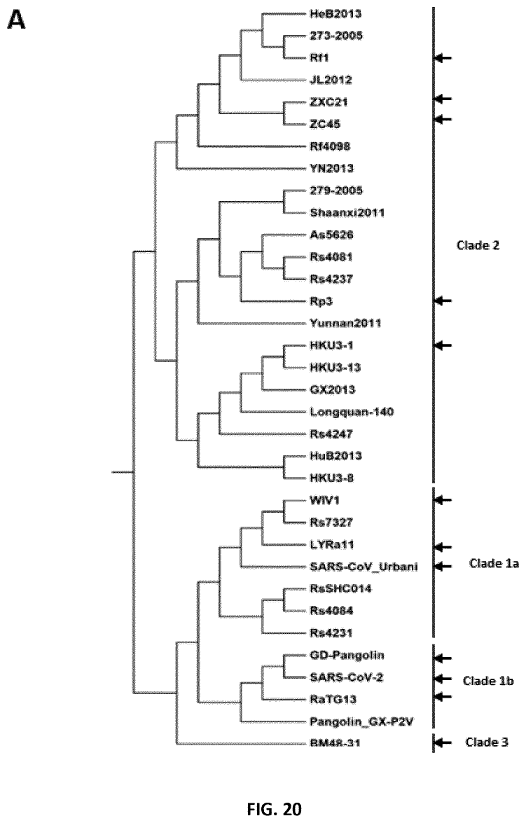

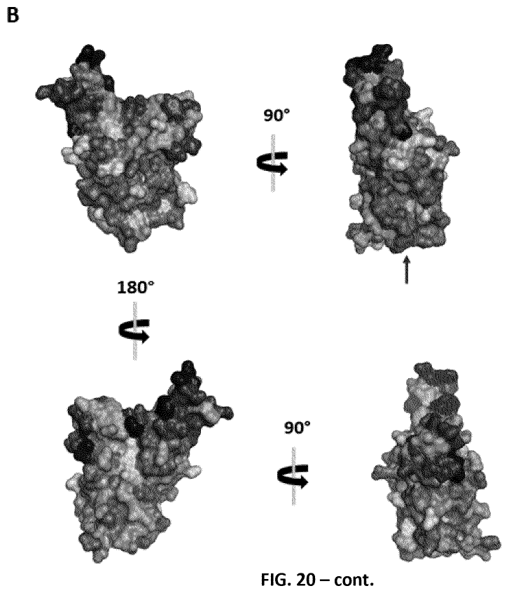

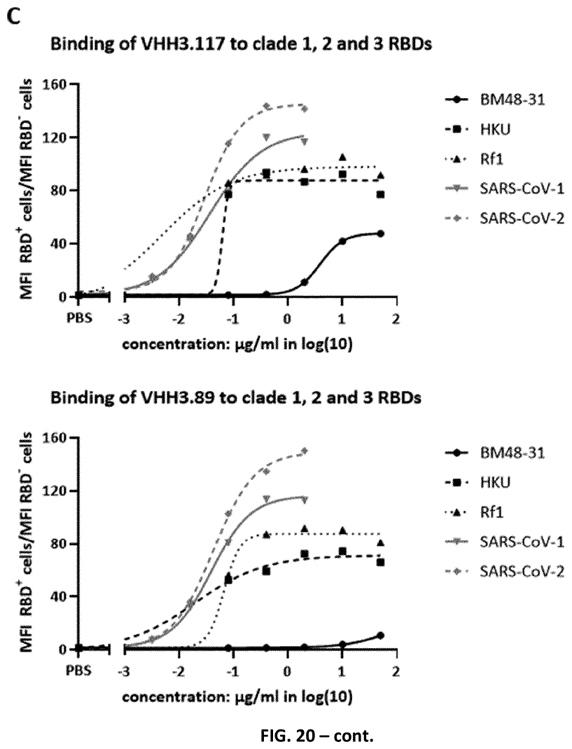

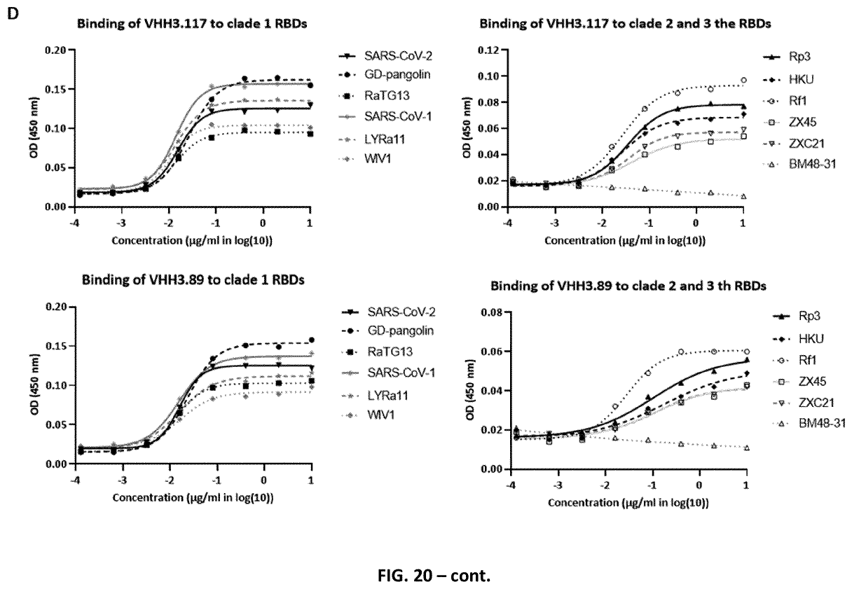

Previous analysis revealed that VHH3.117 can potently bind to the RBD of clade 1 and clade 2 sarbecoviruses and to the RBD of clade 3 BM48-31 sarbecovirus, although with reduced affinity (reference to EP21166835.5 and PCT/EP2022/052919). If VHH3.89 binds the RBD to a site that is highly similar to the binding site of VHH3.117, it should be able to bind the RBD of clade 1 and 2 and to lesser extend to the RBD of clade 3 sarbecoviruses. To test this, we investigated the binding of VHH3.89 to yeast cells expressing the RBD of SARS-COV-2 (clade 1.B), SARS-COV-1 (clade 1.A), HKU3 (clade 1), Rf1 (clade 3) and BM48-31 (clade 3) by flow cytometric analysis (FIG. 20A-C). Both VHH3.117 and VHH3.89 were able to potently bind the RBD of clade 1 and 2 sarbecoviruses and to a marked lower extend to the RBD of the BM48-31 clade 3 virus. In addition, potent binding of both VHH3.117 and VHH3.89 was also observed for a more extended series of clade 1 and 2 viruses when tested by yeast cell ELISA (FIG. 20 D). Taken into account the few site on the RBD that are conserved among clade 1, 2 and 3 sarbecoviruses these results strongly argue that VHH3.89 recognizes an epitope that is highly similar to the VHH3.117 binding site.

### Example 10. Humanization of VHH72-Epitope and VHH3.117-Epitope Binding Agents

In view of developing a pan-specific Corona antibody composition that specifically targets the VHH72- and VHH3.117-epitopes of the RBD Spike protein, a fusion protein of at least two building blocks comprising a VHH has been described herein. Said pan-specific composition is proposed herein in the form of a bispecific construct, wherein the two building blocks are linked directly, via a peptide linker, or via another moiety. For instance VHH building blocks may be fused to an Fc tail, as monovalent, bivalent or bispecific construct, which upon expression in a host will provide for an antibody composition comprising said Fc fusion in dimeric or bivalent format. Said fusion constructs will thus be capable of binding to said binding sites 1 & 2 of the spike protein, as described herein. The development of such VHH-containing pan-specific constructs may require further humanization of the VHH building blocks, and/or of the linker or added moieties, such as of the Fc tail. The skilled person is aware of the methodologies and techniques for humanization as known in the art, and has the knowledge at hand to try out a number of humanization substitutions. The preferred positions and residues for humanization of camelid VHH sequences has been described herein above. In particular for the exemplified fusions as provided herein, we further provide insights and constructs to make humanized variants of the binders described herein.

A number of VHH72 variants have been disclosed previously (Schepens et al. 2021 (Biorxiv doi: https://doi.org/10.1101/2021.03.08.433449 and PCT/EP2021/052885), and may be used in a bi- or multi-valent or -specific format, such as for instance described herein for VHH72-S56A, or humanized variants thereof.

In addition, a further VHH72 humanized variant has been generated and produced in Pichia pastoris, and relates to the SEQ ID NO:90, wherein the VHH72_h1(E1D)(S56A) sequence (SEQ ID NO:4) has been substituted at 5 positions with the amino acid residue corresponding to the same position in VHH3.115, respectively at positions (according to Kabat numbering): R27L, E31D, Y32I, A56G, L97A. Said variants may as well be used for generation of Fc fusions in bivalent or bispecifics formats, for instance in a manner as provided in SEQ ID Nos: 88-89.

Moreover, besides VHH72, other VHHs identified of the same family including and described herein: VHH2.50, VHH3.17, VH3.77, VHH3.115, VHH3.144, and VHHBE4 (as originally identified and presented herein in SEQ ID NOs: 9-14), may be humanized in a similar manner as VHH72. Specifically the framework residues may be substituted with residues that are known to be more ‘human-like’, while the CDR residues are preferably maintained. Humanized variants preferably solely differ in substitutions in the framework residues, preferably one or more of the FR residue positions as listed herein for the particular VHH, and with at least 90% identity of the humanized FR1, 2, 3 or 4, as compared to the original FR1, 2, 3 or 4 sequence. For instance, a VHH3.115 humanized variant is disclosed herein, as part of the Fc fusion shown in SEQ ID NO:94

Further VHH families were described herein specifically binding the VHH72-epitope (or binding site 1) of the spike protein, for which a humanization variant may be envisaged herein as well.

For instance, for VHH3.83 (SEQ ID NO: 6), an example of a humanized variant is disclosed in SEQ ID NO:7 (the ‘VHH3_83 hc’) Comprising the substitutions (according to Kabat numbering) Q1D, K83R, Q108L as compared to the originally identified llama. Similarly, the VHH3.83 family members as depicted in SEQ ID NO:95-98 may be humanized in the same or alternative manner.

Also for the VHH72-epitope binding agents comprising VHH3.36, 3.47, 3.55, 3.35, 3.29, 3.38, and 3.149 (as provided in SEQ ID NOs:15-21), the humanization substitutions listed herein may as well be applicable to provide for humanized variants. Specifically the framework residues may be substituted with residues that are known to be more ‘human-like’, while the CDR residues are preferably maintained. The skilled person may require to confirm the affinity, binding and potency potential of such humanized variants in comparison to the original VHHs as to look for a product with the desired properties.

Multivalent of multispecific formats using said VHH72-epitope binding agent humanized variant are also intended herein, and may be designed as for instance in SEQ ID NO:73-84 and SEQ ID NO:88, 89, and 91-93, or a further humanized variant thereof.

Similarly, for VHH3.117-epitope binding agents, such as VHH3.117, a humanized version named ‘VHH3-117-hc’ as provided in Table 3 constitutes a variant with substitutions Q1D, Q5V, K83R, and Q108L (according to Kabat numbering).

Alternatively, a number of humanized variants are envisaged for characterization of VHH3.117, with the five most prominent candidate residues for humanization substitutions at locations (according to Kabat numbering): Q1, to substitute with D as to avoid pyroglutamate, though the N-terminal substitution may affect the binding properties of VHH3.117 since this is closely located near the epitope region. So a further in-depth analysis of such a variant as to confirm binding potential may be required. Additionally, Q5 replacement with V, K84 replacement with N, K87 with R and Q122 with L are envisaged herein.

Specifically for the original llama-based sequence of VHH3.117 (SEQ ID NO: 22) there may be a requirement for its developability to substitute two methionine residues in CDR3 for obtaining a proper humanized variant. Care should however be taken not to loose or affect its binding capacity, so a sequential substitution approach is recommended.

Furthermore, additional residues may require substitutions for obtaining proper humanized variants, including the Proline at position 39 in framework 2, for instance by an Alanine, the A-Q at position 64-65, and the S-A at positions 77-78, as well as the E82 in framework 3, for instance to be replaced with VK, NT or NA, and Q, resp), and the K on position 119 with Q (according to Kabat numbering).

In addition to humanization of VHH3.117, similar substitutions may be envisaged in the family members including VHH3.92, 3.94, 3.42 and 3.180 (as presented in SEQ ID Nos:24-27).

Specifically the framework residues may be substituted with residues that are known to be more ‘human-like’, while the CDR residues are preferably maintained. Specifically, in the case of humanization of VHH3.117 family members, the CDR sequences as provided in SEQ ID NO: 70 for CDR1, SEQ ID NO:71 for CDR2 and SEQ ID NO:73 for CDR3 should remain as provided herein and the humanized variant solely differs in substitutions in the framework residues, preferably one or more of the FR residue positions as listed herein for the particular VHH, and with at least 90% identity of the humanized FR1, 2, 3 or 4, as compared to the original FR1, 2, 3 or 4 sequence.

Multivalent of multispecific formats using said VHH3.117-epitope binding agent humanized variant are also intended herein, and may be designed as for instance in SEQ ID NO:76-84 and SEQ ID NO:91-93, or a further humanized variant thereof, or alternative fusion combinations similar to those presented herein.

Alternative the VHH3.89 family as described in Example 9 herein may as well be taken in consideration for humanization, similar to the humanization substitutions as typically considered in the art. Preferably, a humanized variant constituting a ‘chimeric’ VHH based on the different family members of the VHH3.89 family may be considered, as to combine the original sequence of CDRs and FRs closest to the human-like sequences. For instance, combine CDR1 of VHH3.89 with the FRs of VHH3.83, which has a double deletion in CDR1 as compared to the other family members.

The expression and purification of said proposed humanized variants can be done according to the methods disclosed herein for cloning, expression and production, and as known to the skilled person. The analysis for selection of the most suitable humanized variants includes (but is not limited to) verification of the specific binding capacity of the humanized VHH as compared to the original VHH for binding to the RBD, for its affinity and for its neutralization potential.

### Example 11. Bispecific Head-to-Tail Fused VHHs Raise the Barrier for Viral Escape

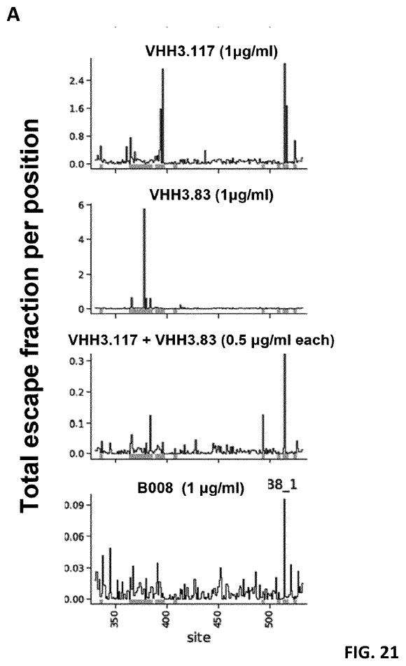

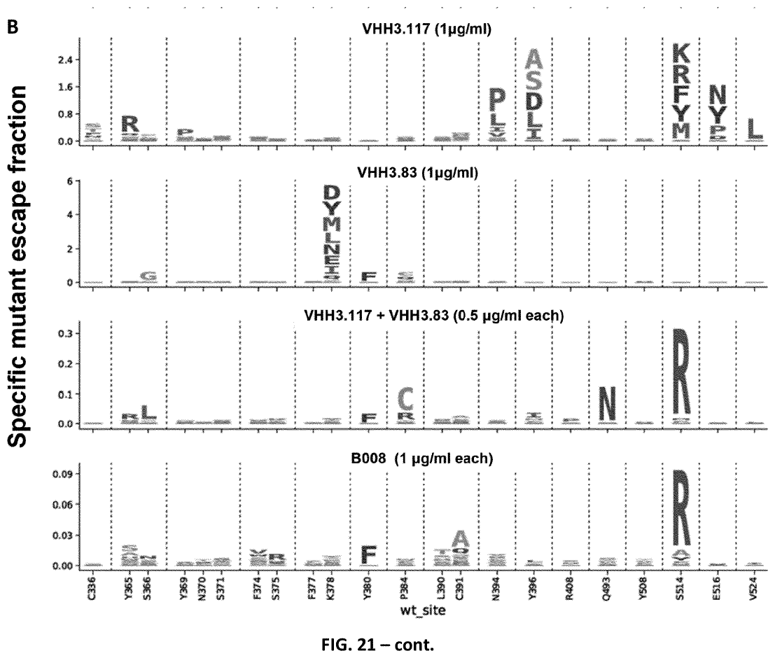

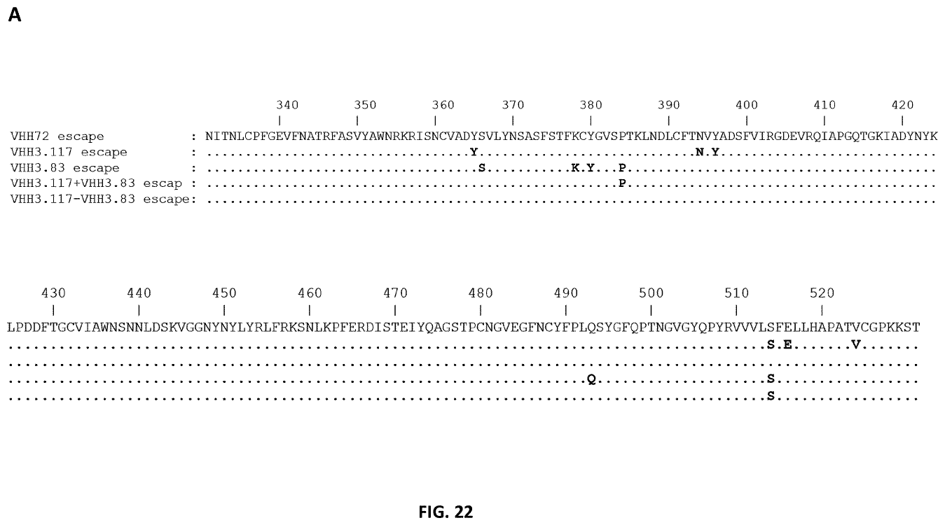

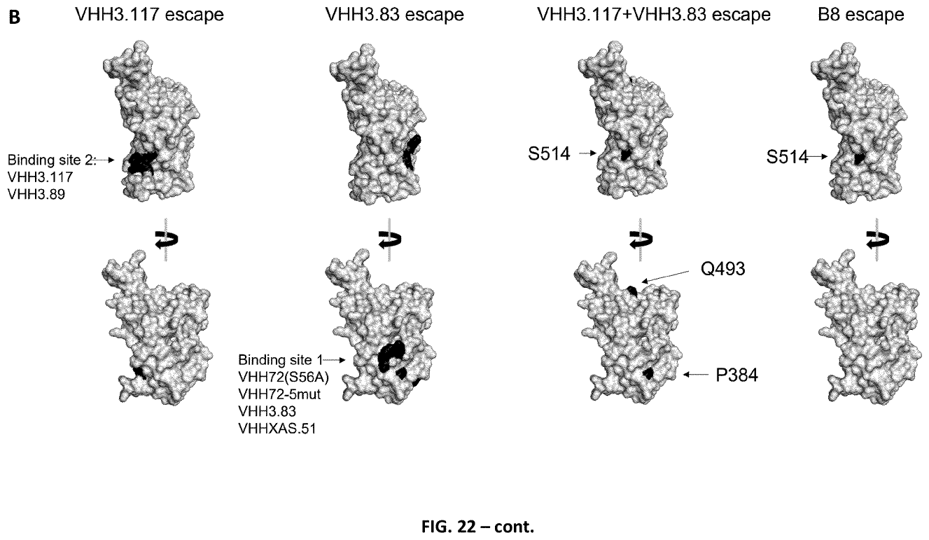

We tested if bispecific constructs in which an ACE2 competing VHH is fused to a VHH that binds the RBD without ACE2 competition can reduce viral escape. Deep mutational scanning (DMS) of RBD mutants was performed for B008, a head-to tail-fusion (SEQ ID NO:80) of VHH3.117 (non ACE2 competitor that binds site 2) and VHH3.83_N85E (ACE2 competitor that binds site 1) and its monovalent parts (PCT/EP2022/052919 and PCT/EP2021/052885). As N85 of VHH3.83 is a putative N-glycosylation site, it was substituted in B008 to a Glutamic Acid. B007 (SEQ ID NO:79) described herein is apart from the N85E mutation identical to B008. Next to a fusion construct also an equimolar cocktail/composition of both VHHs was included. FIG. 21. A shows for each RBD AA position the sum of the fractions that escape binding from the indicated VHHs/VVH constructs/VHH compositions for each of the tested AA substitution at that position. Compared to the individual monovalent VHHs, the cocktail/composition and especially the B008 head-to-tail fusion strongly restrict escape from binding. In agreement, the numbers and escape fraction of the AA positions at which substitutions significantly escaped from VHH binding was higher for the individual VHHs as compared to the head-to-tail fusion B008 (FIGS. 21B and 22). For the VHH3.117 treated samples significant escape was observed at 6 AA positions: Y365, N394, Y396, S514, E516 and V524. All mutations locate at the VHH3.117 binding site that was previously determined by cryo-EM of the Spike/VHH3.117 complex and DMS (PCT/EP2022/052919). For the VHH3.83 treated sample significant escape was observed at 4 AA positions: S366, K378, Y308 and P384. Except for S366, these positions locate at the binding region of VHH3.83 as determined previously by DMS (PCT/EP2021/052885). Mutations at S366 have also been selected by DMS for the epitope mapping of VHH72 and VHH3.38 and VHH3.55 that all bind to a region that is significantly overlapping with the VHH3.83 binding region (PCT/EP2021/052885). S366 is located near the binding site of these VHHs but at the opposite site on the RBD surface. Mutations at that position might impact binding via an allosteric mechanism (PCT/EP2021/052885). Although at lower frequency, escape from binding of the VHH3.117/VHH3.83 cocktail/composition was observed at 3 positions including P384 and S514 for which also escape was observed for VHH3.83 and VHH3.117 individually (FIGS. 21B and 22).

Remarkably, escape was also observed at position Q493 (Q493N) which is located in the Receptor Binding Motive and distant from the two VHH binding sites. A similar observation was made for the REGN10933 and REGN10987 cocktail: DMS analysis revealed strong escape for E406W from the cocktail but not for the individual antibodies (Starr et al. 2021. Science 371:850-854). This AA position on the RBD is distant from the binding sites of both REGN10933 and REGN10987. For the B008 construct, in which VHH3.117 is head-to-tail fused to VHH3.83, significant escape was observed at only 1 position (S514) and very rarely. Escape at that position was also observed for monovalent VHH3.117. These data illustrate that a bispecific VHH fusion construct in which only 1 VHH can compete with ACE2 strongly rises the barrier for viral escape as compared to treatment with the corresponding monovalent VHHs.

### Example 12. Head-to-Tail Fusions of VHH3.83 and VHH3.117 Potently Neutralize Neutralize SARS-COV-2 Variants that Escape from Neutralization by the Individual VHHs

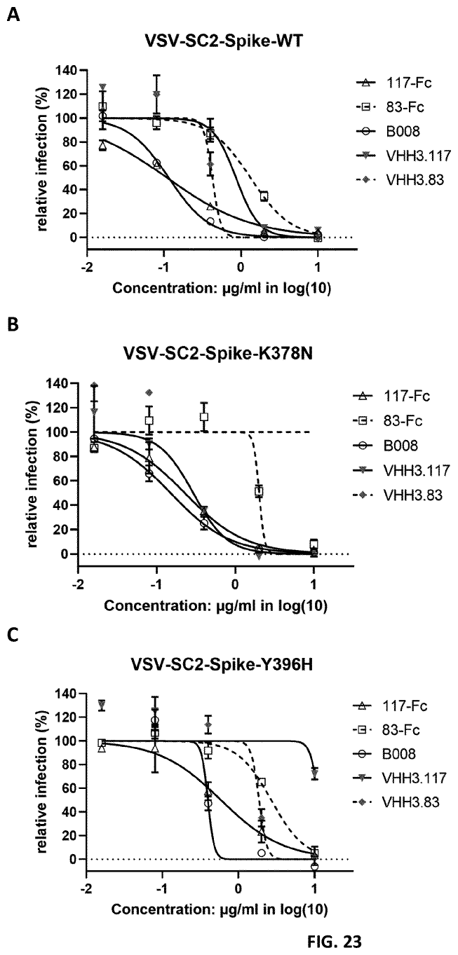

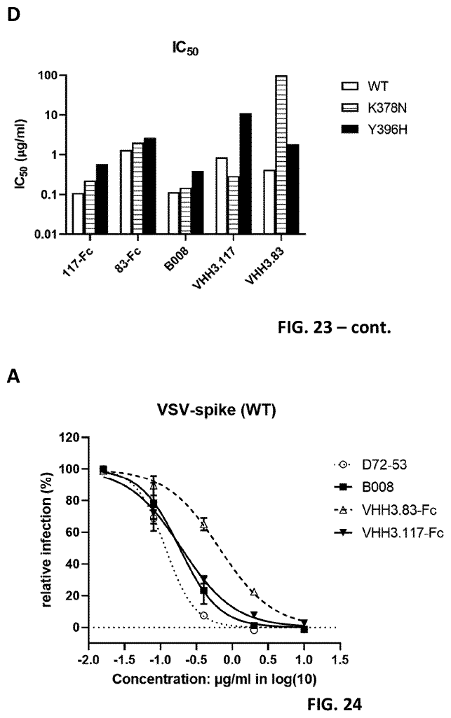

Next, we tested if head-to-tail fusion of 2 VHHs that target respectively, binding site 1 and 2 (i.e. B008 construct as described in Example 11) can overcome escape of SARS-COV-2 variants that escape from neutralization by one of the 2 fused VHHs. Therefore, we generated VSV particles pseudotyped with SARS-COV-2 spike proteins containing the K378N substitution or with SARS-COV-2 spike proteins containing the Y396H substitution that respectively escaped from binding by VHH3.83 and VHH3.117 in DMS analysis. Neutralization assays using these pseudotyped virus particles demonstrated that the K378N and Y396H substitutions significantly impacted neutralization by respectively, VHH3.83 and VHH3.117. In sharp contrast, B008 could still potently neutralize both the K378N and Y396H variant pseudotyped viral particles (FIG. 23).

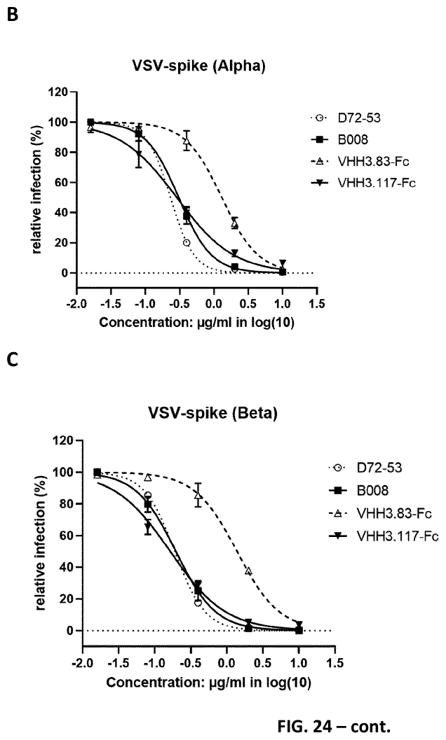

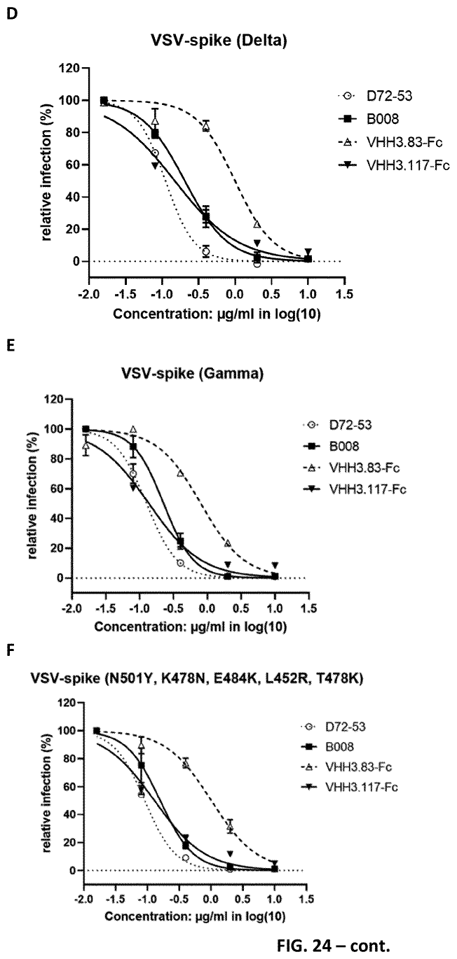

The naturally occurring SARS-COV-2 Alpha, Beta, Delta and Gamma variants harbor RBD mutations distant from the binding site 1 and 2. To confirm that the B008 head-to-tail fusion construct can potently neutralize these variants, neutralization assays were performed using VSV particles that are pseudotyped with spike proteins containing the RBD mutations of the respective SARS-COV-2 variants. In addition, we generated VSV viral particles pseudotyped with a spike protein mutated at all RBD positions that are mutated in those SARS-COV-2 variant viruses. FIG. 24 demonstrates that B008 could potently prevent infection of all these described VSV particles. This indicates that B008 can potently neutralize these SARS-COV-2 variants.

### Example 13. Knob-into-Hole VHH-Fc Constructs Containing VHH3.83 and VHH3.117 can Potently Neutralize SARS-COV-2 Variants that Escape from Neutralization by the Individual VHHs

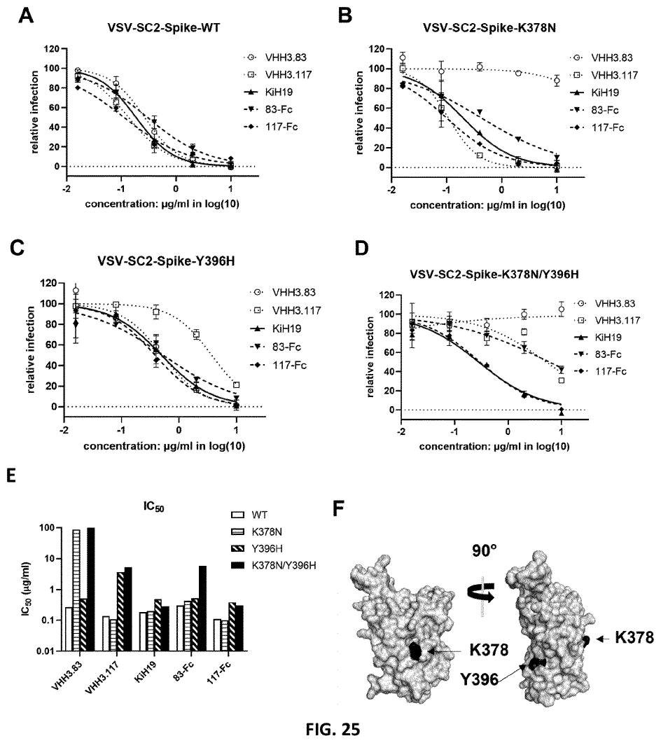

To explore if in addition to head-to-tail fusion also other bispecific formats that contain binding site 1 and 2 targeting VHHs as described herein can potently neutralize SARS-COV-2 and provide a high barrier for viral escape, a knob-into-hole VHH-Fc construct (KiH19, SEQ ID NOs:108 and 107) that contains VHH3.83 and VHH3.117 was generated. Similar to B008, KiH19 was able to potently neutralize viruses that contain the K378N and Y396N substitutions that significantly escape from neutralization by respectively, monovalent VHH3.83 and monovalent VHH3.117 (FIG. 25). To test if such a knob-into-hole VHH-Fc construct can still neutralize viruses that contain escape mutations for both VHHs, we generated a VSV particle pseudotyped with spikes containing both the K378N and the Y396H mutation. Remarkably, KiH19 was able to neutralize this double escape pseudovirus variant almost as efficient as it's parental WT counterpart (FIG. 25). This illustrates that combining a VHH that targets binding site 1 as described herein with a VHH that targets binding site 2 as described herein in a single molecule can vigorously increase the barrier for viral escape.

### Example 14. Knob-into-Hole VHH-Fc Constructs Containing Epitope 1 and 2 Targeting VHHs can Potently Neutralize the SARS-COV-2 Omicron BA.2 Variant

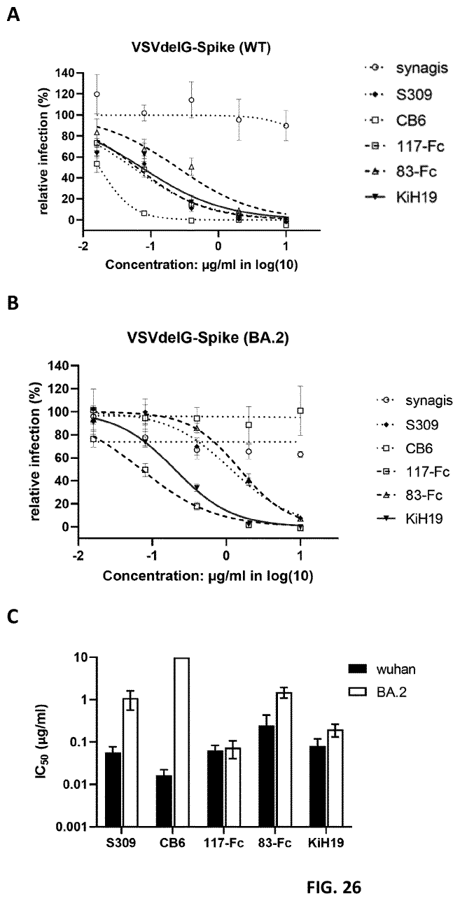

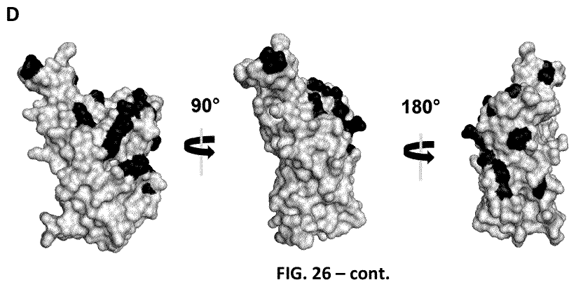

Compared to other SARS-COV-2 variants, the Omicron variants harbor a much larger number of mutations in the spike protein, including in the RBD. Several studies have demonstrated that the neutralizing activity of many monoclonal antibodies that are used under emergency approval or in advanced clinical development is either completely or severely impaired for the Omicron BA.1 and especially Omicron BA.2 variants (Bruel et al. 2022. Nat Med. doi: 10.1038/s41591-022-01792-5.; Cameroni et al. 2022. Nature 602:664-670). Therefore, we investigated the neutralizing activity of KiH19 described in Example 13 and Fc fusions of the two monovalent VHHs that KiH19 comprises (i.e. VHH3.83 and VHH3.117) for virus particles pseudotyped with the Omicron BA.2 variant. The coding sequence of the Omicron BA.2 spike protein from which the 18 C-terminal amino acids were deleted (SEQ ID NO: 130) was ordered as a synthetic nucleotide sequence and cloned into an expression vector to generate VSV particles pseudotyped with Omicron BA.2 spike proteins. FIG. 26D illustrates the positions on the RBD surface that are mutated in the Omicron BA.2 variant. In agreement with what has been reported, the neutralizing activity of the parental antibodies S309 and CB6 of the respectively, approved therapeutic antibodies Sotrovimab and Etesevimab is highly reduced or abolished by the BA.2 mutations (Bruel et al. 2022). A more moderate loss of neutralizing activity was observed for VHH3.83-Fc, whereas the activity of VHH3.117-Fc remained unaffected (FIG. 26). The neutralizing activity of KiH19 was only affected to a minor extend (2.4-fold) (FIG. 26).

### Example 15. Knob-into-Hole VHH-Fc Constructs Containing Epitope 1 and 2 Targeting VHHs Efficient Bind the RBDs of Clade 1, 2 and 3 SARS-COV-2 Variants Including these that are Less Well Recognized by Bivalent Fc Fusion of its 2 VHHs

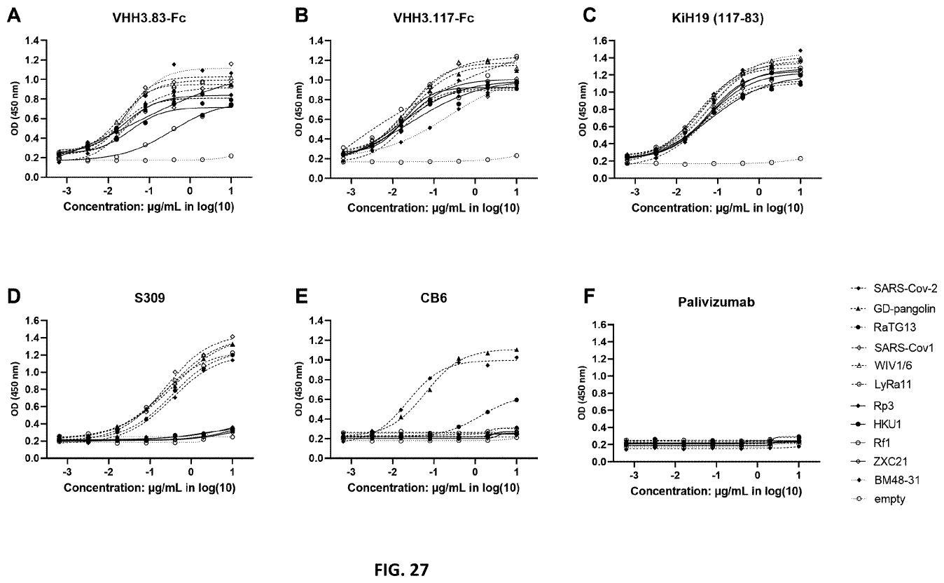

Example 5 revealed that VHH3.117 efficiently recognizes the RBD of a broad panel of Sarbecoviruses but fails to bind the RBD of clade 3 BM48-31 (FIG. 8A). Similarly, VHH3.83 also efficiently recognizes the RBD of a broad panel of Sarbecoviruses but fails to bind the RBD of the clade 2 Rf1 virus (PCT/EP2021/052885). To test if similar to a head-to-tail fusion construct comprising VHH3.83 and VHH3.117 (FIG. 15) a knob-into-hole VHH-Fc efficiently recognized the RBD of clade 1, 2 and 3 Sarbecoviruses including BM48-31 and Rf1, we tested the binding of KiH19 and also of VHH3.83-Fc, VHH3.117-Fc to yeast cells expressing a panel of Sarbecovirus RBDs by ELISA. FIG. 27 illustrates that although VHH3.83-Fc and VHH3.117-Fc can respectively bind Rf1 and BM48-31, the binding to these variants is clearly less efficient as compared to the other RBD variants. In contrast, KiH19 could bind all tested RBD variants with highly similar affinities. As expected, RBD binding by the human monoclonal antibodies S309 and CB6 was restricted to respectively clade 1 and SARS-COV-2 related viruses.

### Example 16. Knob-into-Hole VHH-Fc Constructs Containing Epitope 1 and 2 Targeting VHHs Efficiently Neutralize Authentic Delta Variant SARS-COV-2 Virus

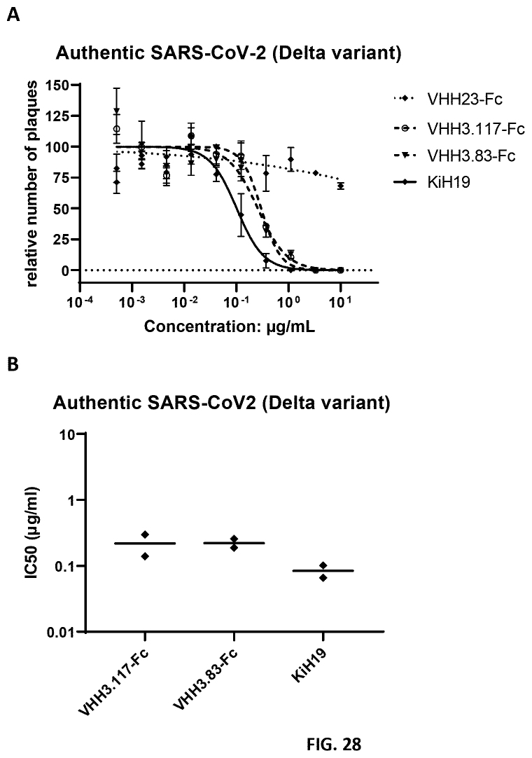

To test if knob-into-hole VHH-Fc constructs containing epitope 1 and 2 targeting VHHs can neutralize authentic SARS-COV-2 viruses we performed a plaque reduction assays using KiH19 and monospecific Fc fusions of VHH3.83 and VHH3.117. FIG. 28 illustrates that KiH19 can efficiently neutralize authentic SARS-COV-2 Delta variant virus. Also VHH3.117-Fc and VHH3.83-Fc can neutralize authentic Delta variant SARS-COV-2 virus but with a somewhat lower efficacy.

### Example 17. Knob-into-Hole VHH-Fc Constructs Comprising the Epitope 1 Binding VHH: VHH72-5Mut and the Epitope 2 Binding VHH: VHH3.89 Efficiently Neutralize WT and VHH72-5Mut Resistant SARS-CoV-2 Variants

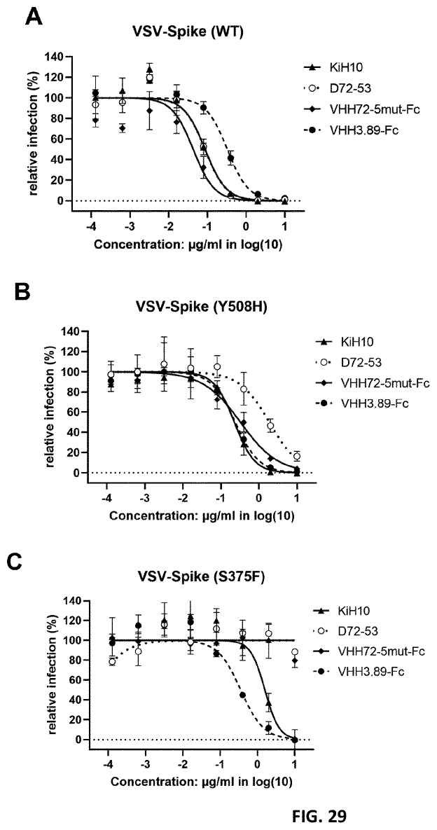

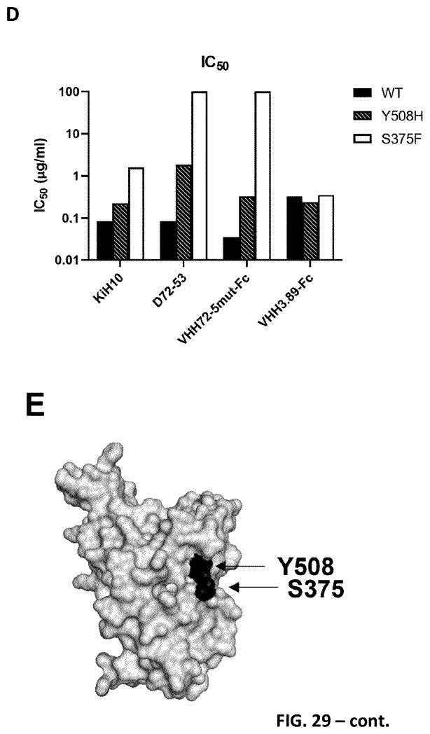

VHH3.115 is highly related to VHH72 but has a higher affinity for SARS-COV-2 RBD and a higher neutralizing activity (PCT/EP2021/052885). Based on the sequence of VHH3.115 a VHH72-variant: VHH72-5mut (SEQ ID NO:90) was generated by introducing 5 substitutions: R27L, E31D, Y31D, S56G and L97A. To partially humanize VHH72-5mut and to avoid possible N-terminal pyroglutamate formation associated charge heterogeneity, the native N-terminal amino acid residue was substituted with an aspartic acid, similar to XVR011 (Schepens et al. 2021. Sci Trans. Med 13:eabi7826). FIG. 29A demonstrates that the Fc fusion of VHH72-5mut (SEQ ID NO:88) is about 2.5-times more potent in neutralizing VSV particle pseudotyped with SARS-COV-2 spikes as compared to the Fc fusion of VHH72-S56A (D72-53=XVR011, SEQ ID NO:105). In addition, VHH72-5mut-Fc is about 5 times more potent in neutralizing VSV particles pseudotyped with the SARS-COV-2 Y508H spike mutant that is partially resistant to D72-53 (FIGS. 29B and E). D72-53 and VHH72-5mut-Fc could not neutralize VSV particles pseudotyped with the SARS-COV-2 S375F variant spike protein (FIGS. 29C and E).

However, when VHH72-5mut was combined with a humanized version of VHH3.89, an epitope 2 binder as described herein, that is different from VHH3.117, into a knob-into-hole VHH-Fc construct (KiH10, SEQ ID NOs: 109 and 110), it could neutralize VSV particles pseudotyped with the SARS-COV-2 S375F variant spike protein with an IC50 of about 1.6 μg/ml (FIG. 29D). Moreover, the neutralizing activity of KiH10 for VSV particles pseudotyped with either WT or S375F variant spikes was in the same range as that of VHH3.89-Fc (SEQ ID NO:104) or VHH72-5mut-Fc (SEQ ID NO:88).

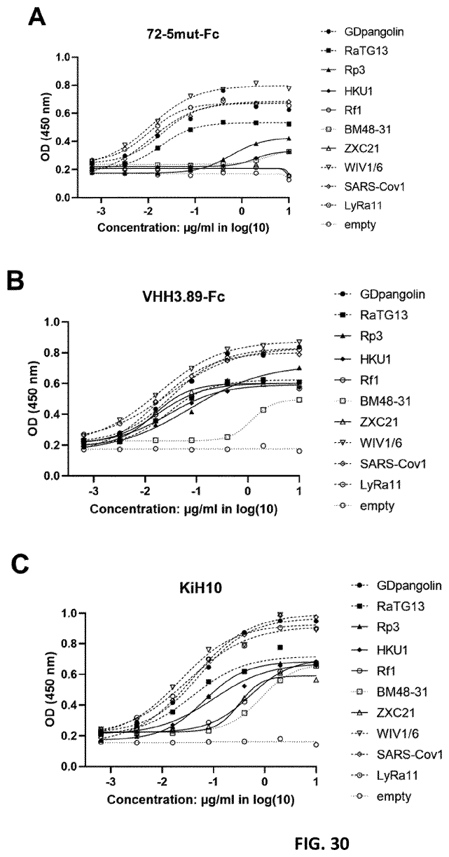

To test if KiH10 can efficiently recognize a broad range of Sarbecoviruses, an ELISA was performed using yeast cells displaying the RBDs of a broad variety of clade 1, 2 and 3 Sarbecoviruses. Whereas VHH72-5mut-Fc could not bind all clade 2 RBDs, KiH10 could (FIGS. 30A and C). In addition, binding of KiH10 to the clade 3 BM48-31 RBD was improved to some extent as compared to VHH3.89-Fc (FIGS. 30B and C).

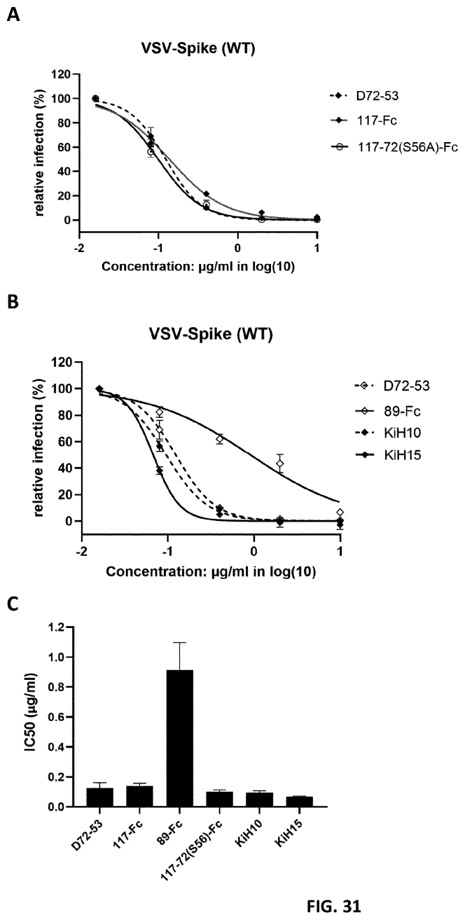

In KiH10 the VHHs are linked to the Fc domain via a (G4S)2 linker and the Fc domain contains the LALA mutations to impair its effector functions. A KiH15 construct (SEQ ID NOs:111 and 112) was produced that is identical to KiH10, except for a (G4S)4 linker spacing the VHH and the Fc domain, the Fc domain has the YTE mutations to prolong half-life in circulation and it contains a non-humanized version of VHH3.89 instead of humanized VHH3.89. Neutralization assays using VSV particles pseudotyped with WT SARS-COV-2 spikes demonstrated that KiH15 had a neutralizing activity that is highly similar to KiH10 (FIGS. 31B and C), which shows that varying the linker and the Fc domain did not impact the neutralizing activity of the knob-into-hole construct.

Collectively, these data illustrate that knob-into-hole VHH-Fc constructs comprising other epitope 1 and epitope 2 targeting VHHs as described herein than VHH3.83 and other than VHH3.117 (which were used in KiH19) can efficiently recognize a broad range of Sarbecoviruses, can potently neutralize SARS-CoV-2 and as such can raise the barrier for viral escape.

### Example 18. VHH1-VHH2-Fc Constructs Comprising Epitope 1 and Epitope 2 Targeting VHHs Recognize a Broad Range of Sarbecoviruses and can Efficiently Neutralize SARS-COV-2

A construct (VHH3.117-VHH72(S56A)-Fc=3.117-72(S56A)-Fc, SEQ ID NO:118) was generated containing head-to-tail fused VHH3.117 and VHH72(S56A), which was fused to an Fc domain. FIG. 31A demonstrates that VHH3.117-VHH72(S56A)-Fc was capable of neutralizing SARS-COV-2 spike pseudotyped VSV particles as efficient as the mono-specific VHH-Fc formats of its corresponding VHHs (FIG. 31C).

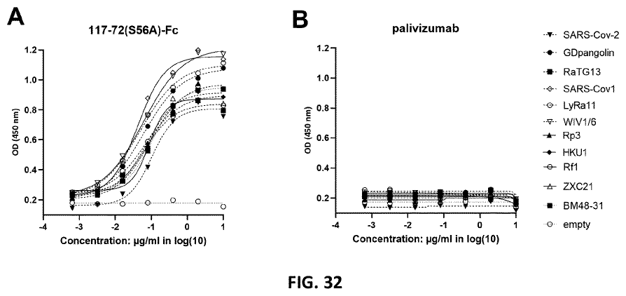

An ELISA was performed using yeast cells displaying a variety clade 1, 2 and 3 RBDs at their surface. FIG. 32 illustrates that VHH3.117-VHH72(S56A)-Fc efficiently recognized the RBDs of clade 1, 2 and 3 Sarbecoviruses.

These data illustrate that also VHH-VHH-Fc bispecific constructs containing epitope 1 and epitope 2 targeting VHHs can broaden the specificity and raise the barrier for viral escape.

### Example 19. Knob-into-Hole VHH-Fc Constructs is a Versatile Format for Bispecific VHHs

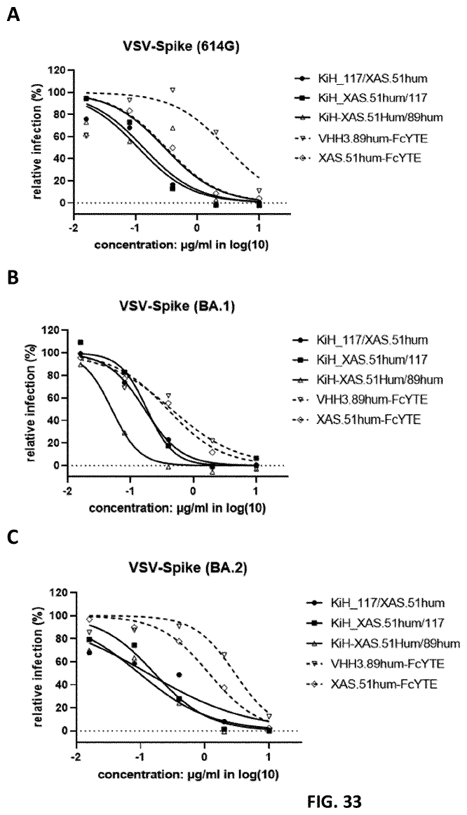

VHH.XAS.51 is a family member of VHH3.83 that was isolated from llama Winter after an additional vaccination series including 1 immunization using the SARS-COV-2 spike-2P protein and 2 subsequent additional immunizations using SARS-COV-2 RBD-SD1. In contrast to VHH3.83, VHH.XAS.51 does not contain an N-glycosylation site (SEQ ID NO:95). Similar to VHH3.83-Fc, VHH.XAS.51hum-Fc potently neutralized the SARS-COV-2 614G variant, but had a somewhat reduced neutralization activity for the Omicron BA.2 variant (FIG. 26 and FIGS. 33A and C).

VHH3.117 was fused to the Fc-hole-chain (SEQ ID NO:115) and combined with humanized VHH.XAS.51 fused to the Fc-knob-chain (SEQ ID NO:116) to generate KiH_VHH3.117/VHH.XAS.51hum (=KiH_117/XAS.51hum, SEQ ID NOs:115 and 116). In addition, also the inverse format (KiH_VHH.XAS.51hum/VHH3.117=KiH_XAS.51hum/117, SEQ ID NOs:113 and 117) was generated. Humanized XAS.51 was also combined with humanized VHH3.89 (Fc-hole-chain) (KiH_VHH.XAS.5hum/VHH3.89hum=KiH_XAS.51hum/89hum; SEQ ID NOs:113 and 114).

These KiH constructs were tested in neutralization assays using VSV particles pseudotyped with the spike protein of either the SARS-COV-2 614G variant (FIG. 33A), the Omicron BA.1 variant (FIG. 33B) or the Omicron BA.2 variant (FIG. 33C). FIG. 33 illustrates that for all three tested SARS-COV-2 variants KiH_VHH3.117/VHH.XAS.51hum and KiH_VHH.XAS.51hum/VHH3.117 had highly similar, potent neutralization activities. This indicates that the neutralizing activity and specificity is not affected by the position of the VHHs in the VHH-Fc knob-into-holes construct.

The neutralization activity of KiH_VHH3.117/VHH.XAS.51hum and KiH_VHH.XAS.51hum/VHH3.117 was less affected by the Omicron mutations than for the mono-specific VHH-Fc format of VHH.XAS.51hum (i.e. VHH.XAS.51hum-Fc(YTE)) (FIG. 33). Similarly, the neutralization activity of KiH_VHH.XAS.51hum/VHH3.89hum was less affected by the Omicron mutations than that of the mono-specific VHH-Fc format: VHH.XAS.51hum-Fc(YTE). This indicates that in a VHH-Fc knob-into-hole format the potential loss of activity of one of the VHHs can consistently be compensated by the unaffected activity of the other VHH (FIG. 33 for VHH3.89-Fc and FIG. 26 for VHH3.117-Fc). The enhanced neutralizing activity of VHH3.89hum-Fc(YTE) for the Omicron BA.1 variant as compared to the 614G variant was paralleled with an enhanced neutralization activity of the KiH_VHH.XAS.51hum/VHH3.89hum (FIG. 33B).

### Example 20. Bispecific VHHa-Fc-VHHb Fusions Comprising Two VHHs that Respectively Target Epitope 1 and 2 can Potently Neutralize SARS-COV-2 Irrespective of Mutations that Affect Neutralization of Epitope 1 Binding VHHs

Bispecific VHH-Fc knob-into-hole formats contain for each epitope a single VHH. Monospecific VHH-Fc constructs containing 2 copies of the same VHHs can retain potent neutralization activity for SARS-COV-2 variants that can escape from the corresponding monovalent VHH (FIG. 23 and FIG. 25). To combine bi-specificity with bivalency for each VHH, tetravalent bispecific VHH1-Fc-VHH2 fusions were generated in which two VHHs respectively recognizing epitope 1 and epitope 2 as described herein are respectively fused to the N- and C-terminus of the Fc domain (FIG. 13E):VHH3.89hum-Fc(YTE)-VHH3.83hum (89hum-FcYTE-83hum, SEQ ID NO: 120) and its inverse counterpart VHH3.83hum-Fc(YTE)-VHH3.89hum (83hum-FcYTE-89hum, SEQ ID NO:121), and VHH.XAS.51hum-FcYTE-VHH3.89hum (XAS.51hum-FcYTE-89hum, SEQ ID NO:119).

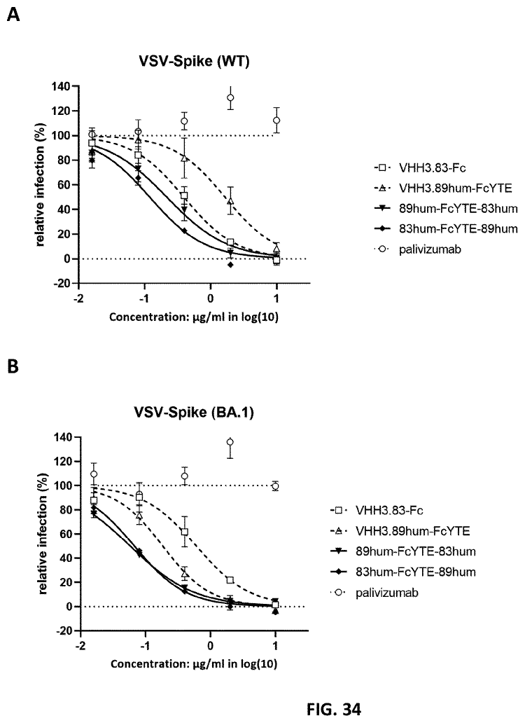

VHH3.89hum-FcYTE-VHH3.83hum and its inverse counterpart VHH3.83hum-FcYTE-VHH3.89hum were tested in a neutralization assay using VSV s particles pseudotyped with the spikes of the SARS-COV-2 614G (FIG. 34A) and Omicron BA.1 (FIG. 34B) variants. FIG. 34 illustrates that VHH3.89hum-FcYTE-VHH3.83hum and its inverse counterpart VHH3.83hum-FcYTE-VHH3.89hum neutralized both virus variants more potently than the mono-specific VHH-Fc fusions of respectively, VHH3.83 and VHH3.89hum. This demonstrates that tetravalent bispecific VHH-Fc-VHH constructs can potently neutralize Sarbecoviruses.

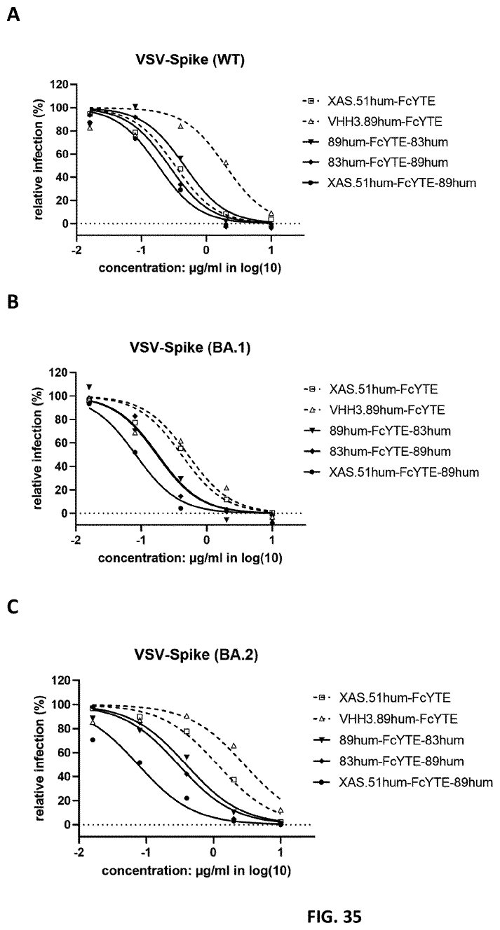

To test if such bispecific VHH-Fc-VHH formats can raise the barrier for escape, their neutralizing activity for the SARS-COV-2 Omicron BA.2 variant was tested. As shown, the neutralizing activity of monospecific VHH-Fc fusions of VHHs that target epitope 1 (VHH3.83 and VHH.XAS.51) for the SARS-CoV-2 Omicron BA.2 variant was reduced (FIG. 26 and FIG. 33). Similar to VHH3.83-Fc (FIG. 26), the neutralizing activity of VHH.XAS51hum-Fc(YTE) (XAS.51hum-FcYTE) was partially affected by the Omicron BA.2 mutations (FIGS. 33A and C), whereas that of VHH3.89hum-Fc(YTE) (89hum-FcYTE) was not (FIGS. 33A and C). Remarkably, the VHH.XAS.51hum-Fc(YTE)-VHH3.89hum (XAS.51hum-FcYTE-89hum) comprising these two VHHs retained full neutralization activity for SARS-COV-2 Omicron BA.2 virus (FIG. 35). Similarly, whereas the neutralization activity of VHH3.83-Fc (83-Fc) was affected (about 6-fold reduction, FIG. 26) by the Omicron BA.2 mutations, VHH3.89hum-FcYTE-VHH3.83hum (89hum-FcYTE-83hum) and its inverse counterpart VHH3.83hum-FcYTE-VHH3.89hum (83hum-FcYTE-89hum) retained full neutralizing activity (FIGS. 35A and C). Also for SARS-COV-2 Omicron BA.1 variant the tested VHH-Fc-VHH constructs retained neutralizing activity similar to that for the historical SARS-CoV-2 614G strain (FIG. 35B). These data demonstrate that bispecific VHH-Fc-VHH fusions targeting epitope 1 and 2 have broad and potent neutralizing activity that raises the barrier for viral escape as compared to the monospecific VHH-Fc constructs targeting these epitopes.

### Example 21. Determination of SARS-COV-2 RBD Amino Acid Positions that can Lose Binding to VHH3.117 and VHH3.89 when Mutated, by Deep Mutational Scanning

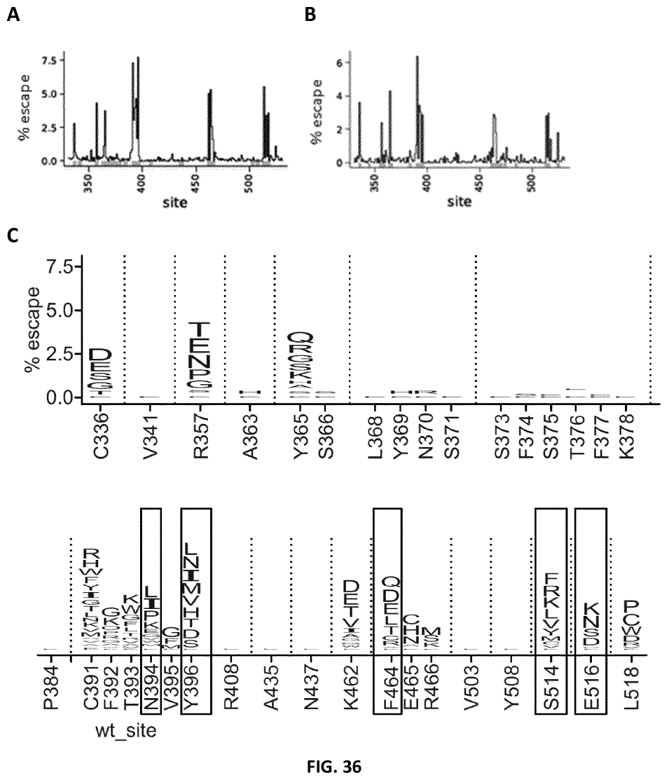

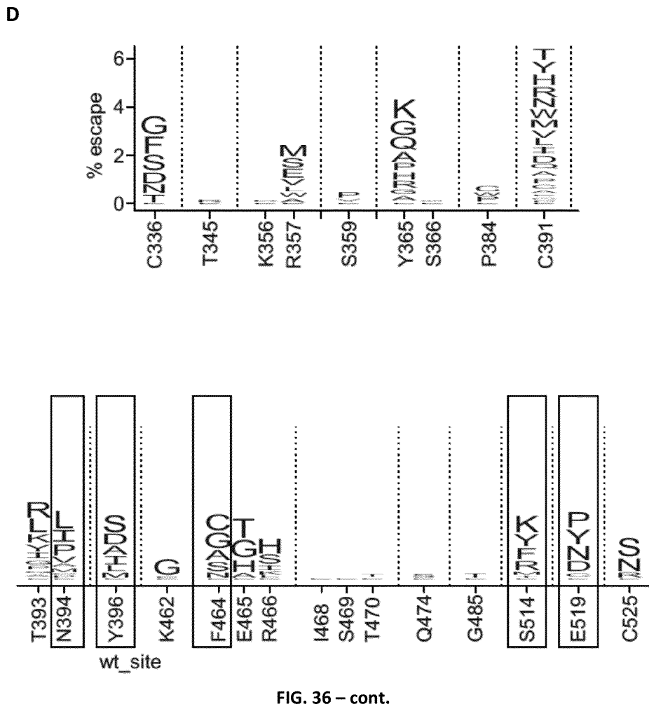

Comparison of the deep mutational scanning signal plotted over the entire length of the RBD shows that the profiles obtained with VHH3.89 and VHH3.117 are highly similar (FIG. 36A-B), demonstrating that these two VHH families are functionally affected in their binding by mutations in a highly similar set of SARS-COV-2 RBD amino acid positions.

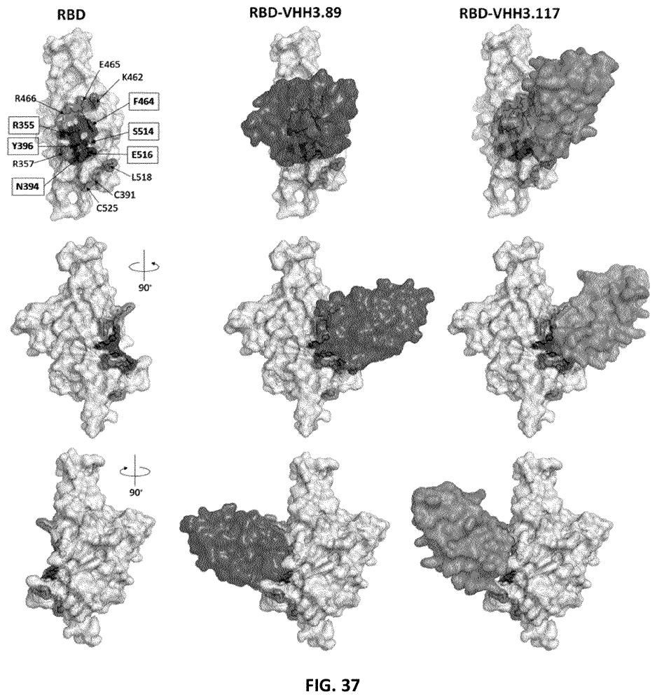

Beyond mutations that affect disulfide bonds that are important for the overall fold integrity of the RBD, the majority of the identified amino acid positions were found to effectively form part of the direct binding contact region of these VHHs with the RBD upon inspection of the corresponding cryoEM-determined structures of the complexes of these VHHs with the SARS-COV-2 spike protein (FIG. 37), allowing to delineate that the core binding contacts for both VHH3.89 and VHH3.117 comprise the positions that are boxed in FIGS. 36C-D. Remaining positions appear to be either more peripheral contacts or local allosteric modulators of the core contact zone.

### Example 22. Cryo-EM Reconstruction of the SARS-COV-2 Spike Protein Trimer in Complex with VHH3.89 and VHH3.117

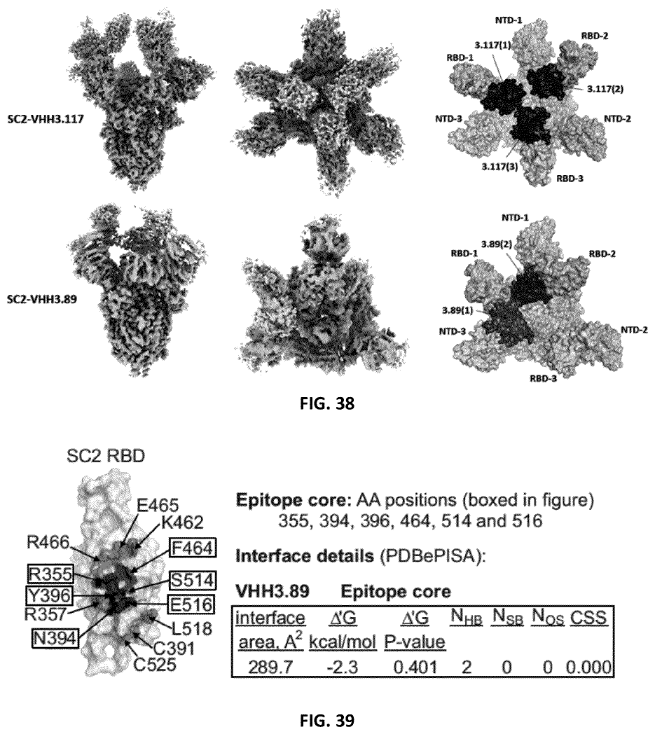

For structure determination of the Spike protein-VHH complexes, VHH3.89 or VHH3.117 were added in 1.3 molar excess to recombinant HexaPro stabilized spike protein (Spike-6P) of the Wuhan SARS-CoV-2 virus. 3 ml of a 0.72 mg/ml SC2-VHH complexes were placed on R2.1 Quantifoil grids prior to snap freezing by plunging the grids into liquid ethane. CryoEM data were collected on a JEOL cryoARM300 electron microscope equipped with Gatan K3 direct electron detector. Single particles were processed using Relion3, resulting in 3D electron potential maps with a nominal resolution of 3.1 Å for the VHH3.117 and VHH3.89 complexes. CryoEM Coulomb potential maps showed unambiguous volumes corresponding to the VHH agents. For the SC2-VHH3.117 complex, all three RBD domains in the SC2 trimer are found in an upright conformation and each have a single copy of VHH3.117 bound (FIG. 38). For the SC2-VHH3.89 complex, all three RBD domains of the SC2 trimer are found in an upright conformation, but with a poor local map density for the RBD of SC2 protomer 3, indicative of a large conformation flexibility in this RBD (FIG. 38). The RBD of SC2 protomers 1 and 2 each have a copy of VHH3.89 bound.

### Example 23. VHH3.117 and VHH3.89-Fc Induce Premature Shedding of the Spike S1 Subunit

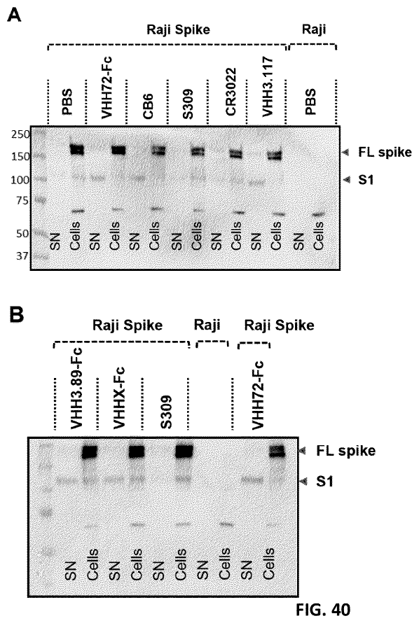

The majority of neutralizing antibodies or nanobodies that target the RBD, neutralize by preventing the binding of the RBD to its receptor ACE2 either by direct binding to the RBM (e.g. CB6) or by sterical hindrance (e.g; VHH72) (Wrapp et all. (2020) Cell 181:1004-1015.e15). Moreover, antibodies that block ACE2 binding are able to induce S1 shedding and as such premature Spike triggering (Wec et al. (2020) Science 369:731-736). Although VHH3.89 and VHH3.117 do neutralize SARS-COV-2 (FIG. 19), they cannot block binding of RBD to ACE2. As an alternative mechanism of neutralization antibodies might induce S1 shedding and consequently premature spike triggering. To investigate if VHH3.117 and VHH3.89-Fc can induce S1 shedding we incubated cells expressing the SARS-COV-2 spike protein with these antibodies and detected S1 shedding into the growth medium by Western blotting using a polyclonal S1 specific antiserum. The ACE2 blocking antibodies CB6 and VHH72-Fc were included as positive controls (Schepens et al. (2021) Sci. Transl. Med. 13). The non-neutralizing antibody CR3022 that does not block ACE2 binding and was shown not to induce S1 shedding was included as negative control (Wec et al. (2020)). In addition, we also included the neutralizing antibody S309 that does not block ACE2 binding (Tortorici et al. (2021) Science 370:950-957). As expected antibodies (CB6 and VHH72-Fc) that can block ACE2 binding to the RBD induced shedding of S1 from the cell surface into the growth medium, as observed by the accumulation of the S1 subunit in the growth medium (SN) and the reduction of what is remained in the cellular fraction as compared to PBS treated cells (FIG. 40A). The two conventional antibodies S309 and CR3022 that cannot block binding of ACE2 to RBD, did also not induce S1 shedding from spike expressing cells (FIG. 40). In sharp contrast to S309 and CR3022 and despite not being able to block binding of ACE2 to RBD, VHH3.117 and VHH3.89-Fc did induce S1 shedding (FIG. 40). Without wishing to be bound by any theory, a possible explanation for the S1 shedding induced by these VHHs is that the common binding region of these VHHs is highly occluded within the spike trimer. As such binding of these VHHs might result in the destabilization of the native spike trimer and consequently promote S1 shedding and premature spike triggering.

### Materials and Methods

Production of VHHs by Pichia pastoris and E. coli.

Small scale production of VHHs in Pichia pastoris is described in (Wrapp et al. 2020 Cell). For the production of VHH in E. coli, a pMECS vector containing the VHH of interest was transformed into WK6 cells (the non-suppressor E coli strain) and plated on an LB plate containing Ampicillin. The next day clones were picked and grown overnight in 2 mL LB containing 100 μg/ml ampicillin and 1% glucose at 37° C. while shaking at 200 rpm. One ml of this preculture was used to inoculate 25 ml of TB (terrific broth) supplemented with 100 μg/ml Ampicillin, 2 mM MgCl₂ and 0.1% glucose and incubated at 37° C. with shaking (200-250 rpm) till an OD₆₀₀ of 0.6-0.9 is reached. VHH production was induced by addition of IPTG to a final concentration of 1 mM. These induced cultures were incubated overnight at 28° C. while shaking at 200 rpm. The produced VHHs were extracted from the periplasm and purified as described in Wrapp et al. In short, the VHHs were purified from the solution using Ni Sepharose beads (GE Healthcare). After elution using 500 mM imidazole the VHH containing flow-through fractions were buffer-exchanged with PBS with a Vivaspin column (5 kDa cutoff, GE Healthcare). The purified VHHs were analyzed by SDS-PAGE and coomassie staining and by intact mass spectrometry.

### Enzyme-Linked Immunosorbent Assay.

Wells of microtiter plates (type II, F96 Maxisorp, Nuc) were coated overnight at 4° C. with 100 ng of recombinant SARS-COV S-2P protein (with foldon), SARS-COV-1 S-2P protein (with foldon), mouse Fc-tagged SARS-COV-2 RBD (Sinobiologicals) or BSA. The coated plates were blocked with 5% milk powder in PBS. Dilution series of the VHHs were added to the wells. Binding was detected by incubating the plates sequentially with either: mouse anti-HA (12CA5, Sigma) combined with HRP conjugated sheep anti-mouse IgG antibody (GE healthcare) or HRP-conjugated rabbit anti-camelid VHH antibodies (Genscript). After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad). Curve fitting was performed using nonlinear regression (Graphpad 8.0).

For the competition assay in which binding of VHHs to monovalent RBD captured by VHH3.117, VHH72-Fc or the human monoclonal antibodies S309, CB6 or palivizumab was tested, ELISA plates (type II, F96 Maxisorp, Nuc) were coated with 100 ng of RBD-SD1 fused to a monovalent human Fc (RBD), VHH3.117, VHH72-Fc (D72-53) or the human monoclonal antibodies in PBS for 16 hours at 4° C. After washing with PBS and then PBS containing 0.1% tween-20, the wells were blocked with PBS containing 5% milk powder for 1 hour at room temperature, 100 ng of monomeric RBD-SD1 fused to a monovalent human Fc was added to the wells and incubated for 1 hour at room temperature. Subsequently, dilution series of HA-tagged VHHs were added to the wells and incubated for 1 hour at room temperature. After washing 2 times with PBS and 3 times with PBS containing 2% milk and 0.05% tween-20 the bound VHHs were detected using a mouse anti-HA-tag antibody (12CA5, Sigma) and an HRP conjugated sheep anti-mouse IgG antibody (GE healthcare). After washing 50 μL of TMB substrate (Tetramethylbenzidine, BD OptETA) was added to the plates and the reaction was stopped by addition of 50 ul of 1 M H₂SO₄. The absorbance at 450 nM was measured with an iMark Microplate Absorbance Reader (Bio Rad). Curve fitting was performed using nonlinear regression (Graphpad 8.0).

### Biolayer Interferometry

The SARS-COV-2 RBD binding kinetics of the VHHs herein were assessed via biolayer interferometry on an Octet RED96 system (ForteBio). To measure the affinity of monovalent VHH variants for RBD, monomeric human Fc-fused SARS-COV-2_RBD-SD1 (Wrapp et al. 2020, supra) at 15 μg/ml was immobilized on anti-human IgG Fc capture (AHC) biosensors (FortéBio) to a signal of 0.35-0.5 nm. Association (120 s) and dissociation (480 s) of duplicate 200 nM VHHs were measured in kinetics buffer. Between analyses, biosensors were regenerated by three times 20 s exposure to regeneration buffer (10 mM glycine pH 1.7). Data were double reference-subtracted and aligned to each other in Octet Data Analysis software v9.0 (FortéBio). Off-rates (kdis) were fit in a 1:1 model.

Competition amongst VHH variants for SARS-COV-2 RBD binding was assessed via biolayer interferometry on an Octet RED96 system (FortéBio). Bivalent VHH72-hFc (50 nM) was immobilized on anti-human IgG Fc capture (AHC) biosensors (ForteBio), followed by capture of antigen RBD-SD1_mFc (200 nM) to saturation. Then, competition with 1 μM VHH variants (protein concentrations calculated by a Trinean DropSense machine, Lunatic chip, after subtraction of the turbidity profile extrapolated from the absorbance spectrum at 320-400 nm) was measured for 600 s. Between analyses, biosensors were regenerated by three times 20 s exposure to regeneration buffer (10 mM glycine pH 1.7). Data were double reference-subtracted and aligned to each other in Octet Data Analysis software v9.0 (FortéBio).

Flow Cytometric Analysis of Antibody Binding to Sarbecovirus RBD Displayed on the Surface of Saccharomyces cerevisiae.

A pool of plasmids, based on the pETcon yeast surface display expression vector, that encode the RBDs of a set of SARS-COV2 homologs was generously provided by Dr. Jesse Bloom (Starr et al. 2020, Cell 182:1295-1310). This pool was transformed to E. coli TOP10 cells by electroporation at the 10 ng scale and plated onto low salt LB agar plates supplemented with carbenicillin. Single clones were selected, grown in liquid low salt LB supplemented with carbenicillin and miniprepped. Selected plasmids were Sanger sequenced with primers covering the entire RBD CDS and the process was repeated until every desired RBD homolog had been picked up as a sequence-verified single clone. Additionally, the CDS of the RBD of SARS-COV2 was ordered as a yeast codon-optimized gBlock and cloned into the pETcon vector by Gibson assembly. The plasmid was transformed into E. coli, prepped and sequence-verified as described above. DNA of the selected pETcon RBD plasmids was transformed to Saccharomyces cerevisiae strain EBY100 according to the protocol by Gietz & Schiestl (Gietz et al. 2007, Nature Protocols 2:1-8 and 31-41) and plated on yeast drop-out medium (SD agar-trp-ura). Single clones were selected and verified by colony PCR for correct insert length. A single clone of each RBD homolog was selected and grown overnight in 10 ml liquid repressive medium (SRaf-ura-trp) at 28° C. These precultures were then back-diluted to 50 ml liquid inducing medium (SRaf/Gal-ura-trp) at an OD₆₀₀ of 0.67/ml and grown for 16 hours before harvest. After washing in PBS, the cells were fixed in 1% PFA, washed twice with PBS, blocked with 1% BSA and stained with VHHs at different concentration. Binding of the antibodies was detected using Alexa fluor 633 conjugated anti-human IgG antibodies (Invitrogen). Expression of the surface-displayed myc-tagged RBDs was detected using a FITC conjugated chicken anti-myc antibody (Immunology Consultants Laboratory, Inc.). Following 3 washes with PBS containing 0.5% BSA, the cells were analyzed by flow cytometry using an BD LSRII flow cytometer (BD Biosciences). Binding was calculated as the ratio between the AF647 MFI of the RBD+(FITC+) cells over the AF647 MFI of the RBD (FITC cells).

Yeast Cell ELISA to Test Antibody Binding to Sarbecovirus RBD Displayed on the Surface of Saccharomyces cerevisiae.

Fixed yeast cells expressing the RBD of various clade 1, 2 and 3 sarbecoviruses were prepared as describe above and coated in ELISA plates in PBS (type II, F96 Maxisorp, Nuc) to obtain about 10-20% confluency. After washing twice with PBS the cells were treated with 3% H₂O₂ for 15 minutes at room temperature to inactivate yeast peroxidases. Subsequently the plates were washed 3 times with PBS and once with PBS containing 0.1% Tween-20. After blocking with 2% BSA for 1 hour, serial dilutions of HA-tagged VHHs were prepared in PBS containing 0.5% BSA and 0.05% Tween-20 and added to the cells and allowed to incubate for 90 minutes. After washing 2 times with PBS and 3 times with PBS containing 0.5% BSA and 0.05% Tween-20 the bound VHHs were detected using a mouse anti-HA-tag antibody (12CA5, Sigma) and an HRP conjugated sheep anti-mouse IgG antibody (GE healthcare). After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad). Curve fitting was performed using nonlinear regression (Graphpad 8.0).

### RBD Competition Assay on Vero E6 Cells.

SARS-COV-2 RBD fused to murine IgG Fc (Sino Biological) at a final concentration of 0.4 μg/mL was incubated with 1 μg/ml of monovalent VHH and incubated at room temperature for 20 min followed by an additional 10 min incubation on ice. RBD without VHH (PBS) and PBS without RBD (no RBD) were used as controls. VeroE6 cells grown at sub-confluency were detached by cell dissociation buffer (Sigma) and trypsin treatment. After washing once with PBS, the cells were blocked with 1% BSA in PBS on ice. All remaining steps were also performed on ice. The mixtures containing RBD and VHHs or VHH-Fc fusions were added to the cells and incubated for 1 h. Subsequently, the cells were washed 3 times with PBS containing 0.5% BSA and stained with an AF647 conjugated donkey anti-mouse IgG antibody (Invitrogen) for 1 h. Following additional 3 washes with PBS containing 0.5% BSA, the cells were analyzed by flow cytometry using an BD LSRII flow cytometer (BD Biosciences).

### CoV Pseudovirus Neutralization Assay.

pCG1 expression vectors for the SARS-COV-2 spike proteins containing the RBD mutations of SARS-COV-2 variants were generated from the pCG1-SARS-2-Sdel18 vector by sequentially introducing the specific RBD mutations by QuickChange mutagenisis using appropriate primers, according to the manufacturer's instructions (Aligent) (coding sequence for spike proteins of SARS-COV-2 Wuhan WT (SEQ ID NO:122), D614 variant (SEQ ID NO:123), Delta variant (SEQ ID No:124), Alpha variant (SEQ ID NO:125), Beta variant (SEQ ID NO:126), Gamma variant (SEQ ID NO:127) and variant combining mutations at all RBD positions that are mutated in the aforementioned variants (SEQ ID ON:128) from which the 18-C terminal amino acids were deleted).

To create Omicron BA.1 and Omicron BA.2 expression vector, the coding sequences of these spike proteins from which the 18 C-terminal amino acids were deleted (SEQ ID NO:129 and 130, respectively) was ordered as a synthetic nucleotide sequence and cloned into an expression vector.

To generate replication-deficient VSV pseudotyped viruses, HEK293T cells, transfected with SARS-COV-1 S or SARS-COV-2 S were inoculated with a replication deficient VSV vector containing eGFP and firefly luciferase expression cassettes (Berger and Zimmer 2011, PloS One 6:e25858). After a 1 h incubation at 37° C., the inoculum was removed, cells were washed with PBS and incubated in media supplemented with an anti-VSV G mAb (ATCC) for 16 h. Pseudotyped particles were then harvested and clarified by centrifugation (Wrapp et al. 2020, Cell 181:1004-1015). For the VSV pseudotype neutralization experiments, the pseudoviruses were incubated for 30 min at 37° C. with different dilutions of purified VHH or with GFP-binding protein (GBP: a VHH specific for GFP). The incubated pseudoviruses were subsequently added to subconfluent monolayers of VeroE6 cells. Sixteen h later the cells were washed once with PBS and cell lysates were prepared using passive lysis buffer (Promega). The transduction efficiency was quantified by measuring the GFP fluorescence in cell lysates using a Tecan infinite 200 pro plate reader. As indicated in the legends the GFP fluorescence was normalized using either the GFP fluorescence of non-infected cells and infected cells treated with PBS or the lowest and highest GFP fluorescence value of each dilution series. Alternatively, infection was quantified by measuring the luciferase activity using promega luciferase assay system and a GloMax microplate luminometer (Promega). The IC₅₀ was calculated by non-linear regression curve fitting, log(inhibitor) vs. normalized response—Variable slope.

### AlphaLISA to Test ACE2/RBD Interaction.

Serial dilutions of VHHs (final concentration ranging between 90 nM-0.04 nM) were made in assay buffer (PBS containing 0.5% BSA and 0.05% Tween-20), and mixed with SARS-COV-2 RBD that was biotinylated through an Avi-tag (AcroBiosystems, Cat nr. SPD-C82E9) (final concentration 1 nM) in white low binding 384-well microtitre plates (F-bottom, Greiner Cat nr 781904). Recombinant human ACE-2-Fc (final concentration 0.2 nM) was added to the mixture. After an incubation of 1 hour at room temperature, donor and acceptor beads were added to a final concentration of 20 μg/mL for each in a final volume of 0.025 mL. RBD was captured on streptavidin coated Alpha Donor beads (Perkin Elmer, Cat nr. 6760002). Human ACE-2-mFc protein (Sino Biological Cat nr. 10108-H05H) was captured on anti-mouse IgG (Fc specific) acceptor beads (Perkin Elmer, Cat nr. AL105C) in an additional incubation of 1 hour at room temperature in the dark. Interaction between beads was assessed after illumination at 680 nm and reading at 615 nm on an Ensight instrument. In contrast to VHH72 and the related VHH3.115, VHH3.42, VHH3.117 nor VHH3.92 could interfere with the RBD/ACE2 interaction even at doses well above their respective neutralization IC50 (54.8 nM, 13.7 nM and 13.55 nM).

### Deep Mutational Scanning

Transformation of deep mutational SARS-COV2 RBD libraries to E. coli: Plasmid preps of two independently generated deep mutational SARS-COV2 RBD libraries in the pETcon vector were generously provided by Dr. Jesse Bloom (Starr et al. 2020, Cell 182, 1295-1310.e20). Ten ng of these preps were transformed to E. coli TOP10 strain via electroporation, and allowed to recover for one hour in SOC medium at 37° C. The transformation mixture was divided and plated on ten 24.5 cm×24.5 cm large bio-assay dishes containing low salt LB medium supplemented with carbenicillin, at an expected density of 100.000 clones per plate. After growing overnight, all colonies were scraped from the plates and resuspended into 300 ml low salt LB supplemented with carbenicillin. The cultures were grown for 2 hours and a half before pelleting. The cell pellet was washed once with sterile MQ, and plasmid was extracted via the QIAfilter plasmid Giga prep kit (Qiagen) according to the manufacturer's instructions. Transformation of deep mutational SARS-COV2 RBD libraries to S. cerevisiae: Ten μg of the resulting plasmid preps were transformed to Saccharomyces cerevisiae strain EBY100, according to the large-scale protocol by Gietz & Schiestl (Gietz et al. 2007, Nature Protocols 2:1-8 and 31-41). Transformants were selected in 100 ml liquid yeast drop-out medium (SD-trp-ura) for 16 hours. Then the cultures were back-diluted into 100 ml fresh SD-trp-ura at 1 OD₆₀₀ for an additional 9 hours passage. Afterwards, the cultures were flash frozen in 1e8 cells aliquots in 15% glycerol and stored at −80° C. Cloning and transformation of WT RBD of SARS-COV2: The CDS of the RBD of SARS-COV2 was ordered as a yeast codon-optimized gBlock and cloned into the pETcon vector by Gibson assembly. The cloning mixture was similarly electroporated into E. coli TOP10 cells, and plasmid was extracted via a Miniprep kit (Promega) according to the manufacturer's instructions. The plasmid was Sanger sequenced with primers covering the entire RBD CDS. Finally, the plasmid was transformed to Saccharomyces cerevisiae strain EBY100, according to the small-scale protocol by Gietz & Schiestl (Gietz et al. 2007, Nature Protocols 2:1-8 and 31-41). Transformants were selected via a yeast colony PCR.

Presorting of deep mutational SARS-COV2 RBD libraries on ACE2: One aliquot of each library was thawed and grown overnight in 10 ml liquid repressive medium (SRaf-ura-trp) at 28° C. Additionally, the control EBY100 strain containing the pETcon plasmid expressing WT RBD from SARS-COV2 was inoculated in 10 ml liquid repressive medium and grown overnight at 28° C. These precultures were then back-diluted to 50 ml liquid inducing medium (SRaf/Gal-ura-trp) at an OD₆₀₀ of 0.67/ml and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer), and stained at an OD₆₀₀ of 8/ml with 9.09 nM hACE2-muFc (Sino Biological) in staining buffer (washing buffer+0.5 mg/ml of Bovine Serum Albumin) for one hour at 4° C. on a rotating wheel. Cells were washed thrice with staining buffer and stained with 1:100 anti-cmyc-FITC (Immunology Consultants Lab), 1:1000 anti-mouse-IgG-AF568 (Molecular Probes) and 1:200 L/D eFluor506 (Thermo Fischer Scientific) for one hour at 4° C. on a rotating wheel. Cells were washed thrice with staining buffer, and filtered over 35 μm cell strainers before sorting on a FACSMelody (BD Biosciences). A selection gate was drawn capturing the ACE2+ cells, such that, after compensation, max. 0.1% of cells of unstained and single stained controls appeared above the background. Approximately 2.5 million ACE2+ cells were collected per library, each in 5 ml polypropylene tubes coated with 2×YPAD+1% BSA.

Sorted cells were recovered in liquid SD-trp-ura medium with 100 U/ml penicillin and 100 μg/ml streptomycin (Thermo Fisher Scientific) for 72 hours at 28° C., and flash frozen at −80° C. in 9 OD₆₀₀ unit aliquots in 15% glycerol.

Nanobody escape mutant sorting on ACE2-sorted deep mutational SARS-COV2 RBD libraries: One ACE2-sorted aliquot of each library was thawed and grown overnight in 10 ml liquid repressive medium (SRaf-ura-trp) at 28° C. Additionally, the control EBY100 strain containing the pETcon plasmid expressing WT RBD from SARS-COV2 was inoculated in 10 ml liquid repressive medium and grown overnight at 28° C. These precultures were then back-diluted to 50 ml liquid inducing medium (SRaf/Gal-ura-trp) at an OD600 of 0.67/ml and grown for 16 hours before harvest.

The cells pellets were washed thrice with washing buffer (1×PBS+1 mM EDTA, pH 7.2+1 Complete Inhibitor EDTA-free tablet (Roche) per 50 ml buffer, freshly made and filter sterile) and stained at an OD₆₀₀ of 8/ml with a specific concentration per stained nanobody in staining buffer (washing buffer+0.5 mg/ml of Bovine Serum Albumin) for one hour at 4° C. on a rotating wheel. Specifically, we stained at 1000 ng/ml for VHH3.117, VHH3.83 and B008 and for the cocktail of VHH3.117+VHH3.83 at 500 ng/ml for each nanobody. Cells were washed thrice with staining buffer and stained with 1:2000 mouse anti-His (Biorad) for 1h30 at 4° C. on a rotating wheel. Cells were washed thrice with staining buffer and stained with 1:100 anti-cmyc-FITC (Immunology Consultants Lab), 1:1000 anti-mouse-IgG-AF568 (Molecular Probes) and 1:200 L/D eFluor506 (Thermo Fischer Scientific) for one hour at 4° C. on a rotating wheel. Cells were washed thrice with staining buffer, and filtered over 35 μm cell strainers before sorting on a FACSMelody (BD Biosciences). Gating was chosen as such that, after compensation, max. 0.1% of cells of the fully stained WT RBD control appeared in the selection gate. Between 150.000 and 350.000 escaped cells were collected per library, each in 5 ml polypropylene tubes coated with 2×YPAD+1% BSA.

Sorted cells were recovered in liquid SD-trp-ura medium supplemented with 100 U/ml penicillin and 100 μg/ml streptomycin (Thermo Fisher Scientific) for 16 hours at 28° C.

DNA extraction and Illumina sequencing of nanobody escape sorted deep mutational SARS-COV2 RBD libraries: Plasmids were extracted from sorted cells using the Zymoprep yeast plasmid miniprep II kit (Zymo Research) according to the manufacturer's instructions, but with the exception of a longer (2 hour) incubation with the Zymolyase enzyme, and with the addition of a freeze-thaw cycle in liquid nitrogen after Zymolyase incubation.

A PCR was performed on the extracted plasmids using KAPA HiFi HotStart ReadyMix to add sample indices and remaining Illumina adaptor sequences using NEBNext UDI primers (20 cycles). PCR samples were purified once using CleanNGS magnetic beads (CleanNA), and once using AMPure magnetic beads (Beckman Coulter). Fragments were eluted in 15 μl 0.1×TE buffer. Size distributions were assessed using the High Sensitivity NGS kit (DNF-474, Advanced Analytical) on a 12-capillary Fragment Analyzer (Advanced Analytical). Hundred bp single-end sequencing was performed on a NovaSeq 6000 by the VIB Nucleomics core (Leuven, Belgium).

### Analysis of Sequencing Data and Epitope Calculation Using Mutation Escape Profiles.

Deep sequencing reads were processed as described by Greaney et al. 2021 (Cell Host Microbe 29:44-57) using the code available at https://github.com/jbloomlab/SARS-COV-2-RBD MAP Crowe antibodies, with adjustments. Briefly, nucleotide barcodes and their corresponding mutations were counted using the dms_variants package (0.8.6). Escape fraction for each barcode was defined as the fraction of reads after enrichment divided by the fraction of reads before enrichment of escape variants. The resulting variants were filtered to remove unreliably low counts and keep variants with sufficient RBD expression and ACE2 binding (based on published data (Starr et al. 2020, Cell 182: 1295-1310). For variants with several mutations, the effects of individual mutations were estimated with global epistasis models, excluding mutations not observed in at least one single mutant variant and two variants overall. The resulting escape measurements correlated well between the duplicate experiments and the average across libraries was thus used for further analysis. To determine the most prominent escape sites for each nanobody, RBD positions were identified where the total site escape was >10× the median across all sites, and was also at least 10% of the maximum total site escape across all positions for a given nanobody.

The AA positions that displayed significant escape were visualized on the RBD surface using PyMol (DeLano 2002. The PyMOL molecular graphics system. DeLano Scientific, San Carlos, CA.).

### Plaque Reduction Assay Using Authentic SARS-COV-2 Delta Variant.

The PRNT experiments were performed at het KULeuven with a SARS-COV-2 isolate from the B.1.617.2 (Delta) lineage. This virus was isolated from oropharyngeal swabs taken from a patient in Belgium (EPI_ISL_2425097; 2021-04-20). Virus stocks were generated by passaging twice on Vero E6 cells. Neutralization by the nanobody constructs was quantified by mixing dilution series of the nanobody constructs, with 100 PFU SARS-COV-2 in DMEM supplemented with 2% FBS. After incubating the mixture at 37° C. for 1h it was added to VeroE6 cell monolayers in 12-well plates and incubated at 37° C. for 1h. Next, the inoculum was replaced with 0.8% (w/v) methylcellulose in DMEM supplemented with 2% FBS. After three days incubation at 37° C., the overlays were removed and the cells were fixed with 3.7% PFA. Subsequently the cells were stained with 0.5% crystal violet. Half-maximum neutralization titers (PRNT50) were defined as the antibody concentration that resulted in a plaque reduction of 50%.

Structure Determination of SC2-VHH3.89 and SC2-VHH3.117 Complexes by cryoEM.

Sample preparation and data collection: For structure determination of the Spike protein-VHH complexes, VHH3.89 or VHH3.117 were added in 1.3 fold molar excess to recombinant HexaPro stabilized spike protein (Spike-6P) of the Wuhan SARS-COV-2 virus. Quantifoil R.2.1 Cu400 holey carbon grids were glow discharged in the ELMO glow discharge system (Corduan Technologies) for 1 min at 11 mA and 0.3 mbar.

The cryo-EM samples were prepared using a CP3 cryoplunger (Gatan). 2 μl of the Spike-6P-VHH complexes at 0.72 mg/ml were applied on a grid and blotted from both sides for 2 s with Whatman No. 2 filter paper at 95% relative ambient humidity, plunge-frozen in liquid ethane at −176° C. and stored in liquid nitrogen prior to data collection.

Cryo-EM images were collected on a JEOL CryoARM 300 microscope at a nominal magnification of 60,000 and the corresponding calibrated pixel size of 0.76 Å, using the Gatan K3 direct electron detector operated in counting mode. For data collection, 3.112 s exposures were dose-fractionated into 60 frames with an electron dose of 1.06 e-Å⁻² per frame. The defocus varied between −0.9 and -2.2 μm. In this way 12915 and 15663 zero-loss micrographs were recorded for the Spike-6P-VHH3.89 and Spike-6P-VHH3.117 complexes, respectively.

EM image processing: The dose-fractionated movies were imported in RELION 4.0 Beta and motion-corrected using RELION's own (CPU-based) implementation of the UCSF motioncor2 program. The Contrast Transfer Function (CTF) parameters were estimated using CTFFIND-4.1.14. References for autopicking were generated by picking a subset of 1000 micrographs using LoG-based auto-picking followed by 2D classification. These references were used for template-based picking of the full datasets, resulting in U.S. Pat. Nos. 1,894,336 and 6,777,098 picked particles for the Spike-6P-VHH3.89 and Spike-6P-VHH3.117 complex, respectively, extracted with a boxsize of 576pixel, binned to 144 pixel. Three consecutive rounds of 2D-classification were performed to clean the particle stack, resulting in 398264 and 239918 remaining particles in the cleaned particle stack for the Spike-6P-VHH3.89 and Spike-6P-VHH3.117 complex, respectively. These remaining particles were re-extracted, binned to 288pixel, and six initial 3D models were generated. Particles belonging to the best 3D class for each complex were re-extracted without binning and subjected to three cycles of consecutive 3D auto-refinement, CTF refinement and classification without alignment. For the Spike-6P-VHH3.89 complex, 222258 particles remained after the final round of classification and 3D auto-refinement, followed by Post-processing resulting in a map with a 3.1 Å nominal resolution according to the 0.143 FSC criterion. For the Spike-6P-VHH3.117 complex 183857 particles remained after the final round of classification and 3D auto-refinement, followed by Post-processing resulting in a 3.1 Å resolution map.

### S1 Shedding Assay

Antibody or VHH was added at a final concentration of 10 μg/ml to 1 million Raji cells expressing either no spike, or SARS-COV-2 spike. The antibody-cell mixture was incubated for 30 min or 1h at 37° C. and 5% CO₂. After incubation, cells were pelleted by centrifugation, supernatant was transferred to a fresh tube and the cell pellet was lysed with RIPA lysis buffer (50 mM Tris-HCl PH 8.0, 100 mM NaCl, 1 mM EDTA, 1 mM EGTA, 0.1% SDS, 1% NP-40). 20 μl samples of supernatant and lysate were separated on 8% SDS-PAGE gels, and electroblotted onto nitrocellulose membranes. Membranes were blocked with 4% milk, stained with rabbit anti-SARS-S1 antibody (1/1000, Sino biologics, 40591-T62) followed by anti-rabbit IgG-HRP (1/2000, GE Healthcare, NA934V) and developed using Pierce™ ECL Western Blotting Substrate (Thermofisher Scientific).

### SEQUENCE LISTING

SEQ ID NO:1: Spike protein Severe acute respiratory syndrome coronavirus 2 (QHQ82464.1)
SEQ ID NO:2: VHH72 amino acid sequence
SEQ ID NO: 3: VHH72-S56A amino acid sequence
SEQ ID NO: 4: VHH72_h1(S56A) humanized variant 1 of VHH72-S56A amino acid sequence
SEQ ID NO:5: VHH72_h1(E1D)(S56A) humanized variant 1(E1D) of VHH72-S56A amino acid sequence
SEQ ID NO:6: VHH3-83 amino acid sequence
SEQ ID NO: 7: VHH3.83 variant VHH3-83-hc amino acid sequence
SEQ ID NO: 8: VHH3.83 variant VHH3-83-hc-N85E amino acid sequence
SEQ ID NO:9: VHH2.50 amino acid sequence
SEQ ID NO:10: VHH3.17 amino acid sequence
SEQ ID NO:11: VHH3.77 amino acid sequence
SEQ ID NO:12: VHH3.115 amino acid sequence
SEQ ID NO:13: VHH3.144 amino acid sequence
SEQ ID NO:14: VHH3BE4 amino acid sequence
SEQ ID NO:15: VHH3.36 amino acid sequence
SEQ ID NO:16: VHH3.47 amino acid sequence
SEQ ID NO:17: VHH3.55 amino acid sequence
SEQ ID NO:18: VHH3.35 amino acid sequence
SEQ ID NO:19: VHH3.29 amino acid sequence
SEQ ID NO:20: VHH3.38 amino acid sequence
SEQ ID NO:21: VHH3.149 amino acid sequence
SEQ ID NO:22: VHH3.117 amino acid sequence
SEQ ID NO:23: VHH3_117-hc amino acid sequence
SEQ ID NO:24: VHH3.92 amino acid sequence
SEQ ID NO:25: VHH3.94 amino acid sequence
SEQ ID NO:26: VHH3.42 amino acid sequence
SEQ ID NO:27: VHH3.180 amino acid sequence

TABLE 5
VHH72 epitope (1) binders

| | FL- | | | | | | |
|---|---|---|---|---|---|---|---|
| | SEQ | | SEQ | | SEQ | | SEQ |
| | ID | | ID | | ID | | ID |
| VHH | NO: | CDR1 | NO: | CDR2 | NO: | CDR3 | NO: |
| VHH72 | 2 | EYAMG | 28 | TISWSGGSTYYTDSVKG | 38 | AGLGTVVSEWDYDYDY | 51 |
| VHH72(S56A) | 3 | EYAMG | 28 | TISWSGGATYYTDSVKG | 39 | AGLGTVVSEWDYDYDY | 51 |
| VHH3.83 | 6 | SYAMG | 29 | AITFNSDATYYADSVKG | 40 | GGNHYNPQYYHDYDKYDH | 52 |
| VHH2.50 | 9 | SIAMG | 30 | TISWSGGSTYYADSVKG | 41 | AGLGTVVSEWDYDYDY | 51 |
| VHH3.17 | 10 | DGAVG | 31 | TVSWNGGGTYFAESVRG | 42 | AGEGTVVSEWDYDYEY | 53 |
| VHH3.77 | 11 | NGAVG | 32 | TVSWNGGGTYFAESVRG | 42 | AGEGTVVSEWDYDYEY | 53 |
| VHH3.115 | 12 | DIAMG | 33 | TISWSGGGTYYAEPVRG | 43 | AGAGTVVSEWDYDYDY | 54 |
| VHH3.144 | 13 | NGAVG | 32 | TVSWNGGGTYYAESVRG | 44 | AGEGTVVSEWDYDYDY | 55 |
| VHH3BE4 | 14 | NGAVG | 32 | TVSWNGGGTYYAESVRG | 44 | AGEGTVVSEWDYDYDY | 55 |
| VHH3.36 | 15 | SYAMG | 29 | AINWGGISVYYADSVKG | 45 | DPKGWSEWDMEY | 56 |
| VHH3.47 | 16 | TYAMA | 33 | AISENDVMRYYADSVKG | 46 | DPKGWSEWDMDY | 57 |
| VHH3.55 | 17 | NYGVG | 34 | AIRWSSISRYYKDSVKG | 47 | DPAGWSEFGMEY | 58 |
| VHH3.35 | 18 | NYGVG | 34 | AIRWSSISRYYKDSVKG | 47 | DPAGWSEFGMEY | 58 |
| VHH3.29 | 19 | SGGMG | 35 | GIGWAGLSSYYLDSVKG | 48 | DDHGWSAAGMDY | 59 |
| VHH3.38 | 20 | NYAMA | 36 | AMFWSGLPKYYADSVKG | 49 | DSRGWSDVGGMDY | 60 |
| VHH3.149 | 21 | SYALG | 37 | AINWFGAPTYYADSVKG | 50 | DSKGWDPQDMDY | 61 |
| VHH72-5mut | 90 | DIAMG | 33 | TISWSGGGTYYTDSVKG | 144 | AGAGTVVSEWDYDYDY | 54 |
| VHH4.1XAS51 | 95 | NYAMG | 141 | AITFNSDATYYADSVKG | 145 | GGNHYNPQYYHDYDKYDY | 146 |
| VHH4.1XAS58 | 96 | AFSIG | 142 | AITFNSDATYYADSVKG | 145 | GGNHYNPQYYHDYDKYDH | 52 |
| VHH4.1XAS31 | 97 | SDYAVA | 143 | AITFNSDATYYADSVKG | 145 | GGNHYNPQYYHDYDKYDH | 52 |
| VHH4.1XAS43 | 98 | NYAMG | 141 | AITFNSDATYYADSVKG | 145 | GGNHYNPQYYHDYDKYDH | 52 |

TABLE 6
VHH3.117 epitope (2) binders.

| | FL- | | | | | | |
|---|---|---|---|---|---|---|---|
| | SEQ | | SEQ | | SEQ | | SEQ |
| | ID | | ID | | ID | | ID |
| VHH | NO: | CDR1 | NO: | CDR2 | NO: | CDR3 | NO: |
| VHH3.117 | 22 | ISDMGW | 62 | TITKTGSTNYADSAQG | 64 | WLPYGMGPDYYGME | 68 |
| VHH3.92 | 24 | ISDMGW | 62 | TITKTGNTNYADSAQG | 65 | WLPYGMGPDYYGME | 68 |
| VHH3.94 | 25 | ISDMGW | 62 | TITKSGSTNYANSAQG | 66 | WLPYGMGPDYYGME | 68 |
| VHH3.42 | 26 | INDMGW | 63 | TITKTGSTNYADSVKG | 67 | WLPYGMGPDYYGME | 68 |
| VHH3.180 | 27 | ISDMGW | 62 | TITKTGSTNYADSAQG | 64 | WLLYGMGPDYYGME | 69 |
| VHH3.89 | 85 | YYAIG | 131 | RIDSSDGSTYYADSVKG | 133 | DPIIQGRNWYWT | 135 |
| VHH3_183 | 86 | YYAIG | 131 | RIESSDGSTYYADSVKG | 134 | DPIIQGSSWYWT | 136 |
| VHH3C_80 | 87 | DYDVG | 132 | RIDSSDGSTYYADSVKG | 133 | DPIIRGHNWYWT | 137 |

SEQ ID NO:70: amino acid consensus sequence of CDR1 for the VHH3.117 family, wherein X (Xaa) at position 2 is S or N
IXDMGW
SEQ ID NO:71: amino acid consensus sequence of CDR2 for the VHH3.117 family, wherein X (Xaa) at position 5 is T or S, X (Xaa) at position 7 is S or N, X (Xaa) at position 12 is D or N, X (Xaa) at position 14 is A or V, and X (Xaa) at position 15 is Q or K.
TITKXGXTNYAXSXXG
SEQ ID NO:72: amino acid consensus sequence of CDR3 for the VHH3.117 family, wherein X (Xaa) at position 3 is P or L.
WLXYGMGPDYYGME
SEQ ID NO: 73: homobivalent VHH amino acid sequence encoded by construct pX-B001_GS-VHH72-h1-E1D-S56A_(G4S)6_VHH72-h1-E1D-S56A_His8
SEQ ID NO:74: homobivalent VHH amino acid sequence encoded by construct pX-B002_GS-VHH72-h1-E1D-S56A_(G4S)_VHH72-h1-E1D-S56A_His8
SEQ ID NO:75: homobivalent VHH amino acid sequence encoded by construct pX-B003_GS-VHH72-h1-E1D-S56A_(G4S)₄_VHH72-h1-E1D-S56A_His8
SEQ ID NO:76: Bispecific VHH amino acid sequence encoded by construct pX-B004_GS-VHH3-117-hc_(G4S)6_VHH72-h1-E1D-S56A_His8
SEQ ID NO:77: Bispecific VHH amino acid sequence encoded by construct pX-B005_GS-VHH3-117-hc_(G4S)_VHH72-h1-E1D-S56A_His8
SEQ ID NO:78: Bispecific VHH amino acid sequence encoded by construct pX-B006_GS-VHH3-117-hc_(G4S)₄_VHH72-h1-E1D-S56A_His8
SEQ ID NO: 79 Bispecific VHH amino acid sequence encoded by construct pX-B007_GS-VHH3-117-hc_(G4S)6_VHH3-83-hc_His8
SEQ ID NO: 80: Bispecific VHH amino acid sequence encoded by construct pX-B008_GS-VHH3-117-hc_(G4S)6_VHH3-83-hc-N85E_His8
SEQ ID NO: 81: Bispecific VHH amino acid sequence encoded by construct pX-B009_GS-VHH3-117-hc_(G4S)_VHH3-83-hc_His8
SEQ ID NO: 82: Bispecific VHH amino acid sequence encoded by construct pX-B010_GS-VHH3-117-hc_(G4S)₄_VHH3-83-hc_His8
SEQ ID NO: 83: Bispecific VHH amino acid sequence encoded by construct pX-B011_GS-VHH3-117-hc_(G4S)_VHH3-83-hc-N85E_His8
SEQ ID NO: 84: Bispecific VHH amino acid sequence encoded by construct pX-B012_GS-VHH3-117-hc_(G4S)₄_VHH3-83-hc-N85E_His8
SEQ ID NO:85: VHH3.89 amino acid sequence
SEQ ID NO: 86: VHH3_183 amino acid sequence
SEQ ID NO:87: VHH3C_80 amino acid sequence
SEQ ID NO: 88: Bivalent-Fc construct VHH72_h1_E1D_R27L_E31D_Y32I_S56G_L97A-10×GS-hIgG1_EPKSCdel_LALA_K447del
SEQ ID NO: 89: Bivalent-Fc construct VHH72-h1-E1D-R27L-E31D-Y32I-S56G-L97A_(GGGGS)×2_hIgGhingeEPKSCdel_hIgGFc_N297A_Gsdel
SEQ ID NO:90: VHH72-h1-E1D-R27L-E31D-Y32I-S56G-L97A amino acid sequence
SEQ ID NO:91: Bispecific VHH amino acid sequence of construct pX-B014_GS-VHH3_117-hc_(G4S)6_VHH72-h1-E1D-R27L-E31D-Y32I-S56G-L97A_His8
SEQ ID NO:92: Bispecific VHH amino acid sequence of construct pX-B017_GS-VHH3_117-hc_(G4S)_VHH72-h1-E1D-R27L-E31D-Y32I-S56G-L97A_His8
SEQ ID NO:93: Bispecific VHH amino acid sequence of construct pX-B018_GS-VHH3_117-hc_(G4S)₄_VHH72-h1-E1D-R27L-E31D-Y32I-S56G-L97A_His8
SEQ ID NO: 94: Bivalent VHH-Fc amino acid sequence VHH3.115-h1-E1D_(GGGGS)×2_hIgGhingeEPKSCdel_hIgGFc_N297A_Gsdel
SEQ ID NO: 95: VHH4.1XAS51 amino acid sequence
SEQ ID NO: 96: VHH4.2XAS58 amino acid sequence
SEQ ID NO: 97: VHH4.2XAS31 amino acid sequence
SEQ ID NO: 98: VHH4.2XAS43 amino acid sequence
SEQ ID NO: 99: SARS-COV-2 RBD: AA 381-531 of SEQ ID NO:1
SEQ ID NO: 100: SARS-COV-2 RBD: AA 333-516 of Wuhan-Hu-1 isolate
SEQ ID NO: 101: SARS-COV-2 RBD: AA 331-531 of SEQ ID NO:1

TABLE 7
Monospecific bivalent VHH-Fc fusions

| | | SEQ | | | |
|---|---|---|---|---|---|
| | | ID | | lgG hinge | |
| | Full name | NO: | Linker | format | Fc domain |
| VHH3.11 | VHH117_Q1D-10xGS- | 102 | (G₄S)₂ | hlgG1_EPKS | hlgG1_LALA |
| 7-Fc | hlgG1_EPKSCdel_LALA_K447del | | | Cdel | Kdel |
| VHH3.83- | VHH83_Q1D-N85E_10xGS- | 103 | (G₄S)₂ | hlgG1_EPKS | hlgG1_LALA |
| Fc | hlgG1_EPKSCdel_LALA_K447del | | | Cdel | Kdel |
| Vhh72- | VHH72_h1_E1D_R27L_E31D_Y32I_S56G_L | 88 | (G₄S)₂ | hlgG1_EPKS | hlgG1_LALA |
| 5mut-Fc | 97A-10xGS-hlgG1_EPKSCdel_LALA_K447del | | | Cdel | Kdel |
| VHH3.89- | VHH89_Q1D-10xGS- | 104 | (G₄S)₂ | hlgG1_EPKS | hlgG1_LALA |
| Fc | hlgG1_EPKSCdel_LALA_K447del | | | Cdel | Kdel |
| D72-53 | VHH72_h1_E1D_S56A-10xGS- | 105 | (G₄S)₂ | hlgG1_EPKS | hlgG1_LALA |
| | hlgG1_EPKSCdel_LALA_K447del | | | Cdel | Kdel |
| VHH23-Fc | VHH23-GS(G4S)2-hlgG1hinge-hlgG1 | 106 | GS(G₄S)₂ | hlgG1 | hlgG1 |

TABLE 8
Knob-into-hole VHH-Fc fusions

| | | SEQ | | | |
|---|---|---|---|---|---|
| | | ID | | lgG hinge | |
| | Fc chain | NO: | Linker | format | Fc domain |
| KiH19 = | VHH3.117-holeFc | 107 | (G₄S)₂ | Fc_EPKS | hlgG1_LALA |
| VHH117/VHH83-10GS-KiH | | | | Cdel | Kdel |
| | VHH83_Q1D_N85E-knobFc | 108 | (G₄S)₂ | Fc EPKS | hlgG1_LALA |
| | | | | Cdel | Kdel |
| KIH10 = | hole Fc of KiH10 | 109 | 10GS | Fc_EPKS | hlgG1_LALA |
| VHH72_5mut/VHH89Hum- | VHH72_h1_E1D_R27L_E31D | | | Cdel | Kdel |
| KiH | Y32I_S56G_L97A-holeFc | | | | |
| | VHH89_Q1D_Hum-knobFc | 110 | 10GS | Fc_EPKS | hlgG1_LALA |
| | | | | Cdel | Kdel |
| KIH15 = | hole Fc of KiH15 | 111 | 20GS | Fc_EPKS | hlgG1I_LALA |
| VHH72_5mut/VHH89- | VHH72_h1_E1D_R27L_E31D | | | Cdel | YTE_Kdel |
| 20GS-Fc_LALA_YTE-KiH | Y32I_S56G_L97A-holeFc | | | | |
| | VHH89_Q1D-knobFc | 112 | 20GS | Fc_EPKS | hlgG1I_LALA |
| | | | | Cdel | _YTE_Kdel |
| KIH_VHH.XAS.51hum/ | VHH.xas51Hum-holeFc | 113 | (G₄S)₂ | Fc_EPKS | hlgG1_YTE |
| VHH3.89hum = | | | | Cdel | Kdel |
| VHH.xas51Hum/VHH89Hu | VHH89Hum-knobFc | 114 | (G₄S)₂ | Fc_EPKS | hlgG1_YTE |
| m-10GS- | | | | Cdel | Kdel |
| hlgG1_EPKSCdel_YTE_K44 | | | | | |
| 7del_KiH | | | | | |
| KIH_VHH3.117/ | VHH3.117-hole Fc | 115 | (G₄S)₂ | Fc_EPKS | hlgG1_YTE |
| VHH.XAS.51hum = | | | | Cdel | Kdel |
| VHH117/VHHxas51Hum- | VHH.XAS.51hum-knobFc | 116 | (G₄S)₂ | Fc EPKS | hlgG1_YTE |
| 10GS- | | | | Cdel | Kdel |
| hlgG1_EPKSCdel_YTE_K44 | | | | | |
| 7del_KiH | | | | | |
| KiH_VHH.XAS.51hum/VH | VHH.XAS.51hum-hole Fc | 113 | (G₄S)₂ | Fc_EPKS | hlgG1_YTE |
| H3.117 = | | | | Cdel | Kdel |
| VHHxas51Hum/VHH117- | VHH3.117-knobFc | 117 | (G₄S)₂ | Fc_EPKS | hlgG1_YTE |
| 10GS- | | | | Cdel | Kdel |
| hlgG1_EPKSCdel_YTE_K44 | | | | | |
| 7del_KiH | | | | | |

TABLE 9
VHH-VHH-Fc fusions

| | | SEQ | | | |
|---|---|---|---|---|---|
| | | ID | | lgG hinge | |
| | Full name | NO: | Linker | format | Fc domain |
| VHH3.117- | VHH117_Q1D-15GS- | 118 | (G₄S)₂ | hlgG1_EPKSCdel | hlgG1_LALA_Kdel |
| VHH72(S56A)- | VHH72_Q1D_S56A-10GS- | | | | |
| Fc | Fc_EPKSCdel_LALA_Kdel | | | | |

TABLE 10
VHH-Fc-VHH fusions

| | | SEQ | | | |
|---|---|---|---|---|---|
| | | ID | | lgG hinge | |
| | Full name | NO: | Linker | format | Fc domain |
| VHH.XAS.51hum- | VHHxas51Hum-10GS- | 119 | (G₄S)₂ | hlgG1_EPKSCdel | hlgG1_LALA_Kdel |
| FcYTE- | hlgG1_EPKSCdel_YTE_K447del- | | | | |
| VHH3.89hum | 10GS-VHH89Hum_D1E | | | | |
| VHH3.89hum- | VHH89Hum-10GS- | 120 | (G₄S)₂ | hlgG1_EPKSCdel | hlgG1_LALA_Kdel |
| FcYTE- | hlgG1_EPKSCdel_YTE_K447del- | | | | |
| VHH3.83hum | 10GS-VHH83Hum | | | | |
| VHH3.83hum- | VHH83Hum-10GS- | 121 | (G₄S)₂ | hlgG1_EPKSCdel | hlgG1_LALA_Kdel |
| FcYTE- | hlgG1_EPKSCdel_YTE_K447del- | | | | |
| VHH3.89hum | 10GS-VHH89Hum_D1E | | | | |

SEQ ID NO: 122: SARS-COV-2 Wuhan WT Spike_del18: SARS-COV-2 Wuhan WT spike protein from which the 18 C-terminal amino acids were deleted. >SEQ ID NO: 123: SARS-COV-2 D614G Spike_del18: SARS-COV-2 D614G spike protein from which the 18 C-terminal amino acids were deleted.
SEQ ID NO: 124: SARS-COV-2 Spike_del18_L452R/T478K: SARS-COV-2 Delta variant spike protein from which the 18 C-terminal amino acids were deleted.
SEQ ID NO: 125: SARS-COV-2 Spike_del18_N501Y: SARS-COV-2 Alpha variant spike protein from which the 18 C-terminal amino acids were deleted.
SEQ ID NO: 126: SARS-COV-2 Spike_del18_N501Y/K417N/E484K: SARS-COV-2 Beta variant spike protein from which the 18 C-terminal amino acids were deleted.
SEQ ID NO: 127: SARS-COV-2 Spike_del18_N501Y/K417T/E484K: SARS-COV-2 Gamma variant spike protein from which the 18 C-terminal amino acids were deleted.
SEQ ID NO: 128: SARS-COV-2 Spike_del18 (N501Y/K417N/E484K/L452R/T478K): SARS-COV-2 N501Y/K417N/E484K/L452R/T478K spike protein from which the 18 C-terminal amino acids were deleted.
SEQ ID NO: 129: SARS-COV-2 Omicron BA.1 Spike_del18: SARS-COV-2 Omicron BA.1 variant spike protein from which the 18 C-terminal amino acids were deleted.
SEQ ID NO: 130: SARS-COV-2 Omicron BA.2 Spike_del18SARS-COV-2 Omicron BA.2 variant spike protein from which the 18 C-terminal amino acids were deleted.

1. A composition comprising one or more agents specifically binding the Corona virus Spike protein, wherein the one or more agents comprise one or more first immunoglobulin single variable domains (ISVDs) binding to the amino acid residues Y369, F377, and K378 of the SARS-COV-2 spike protein as depicted in SEQ ID NO: 1, and one or more second ISVDs binding to at least one or more of the residues T393, N394, V395, or Y396 of the SARS-CoV-2 spike protein as depicted in SEQ ID NO:1,
wherein the one or more first ISVDs comprise the complementarity determining regions (CDRs) as depicted in any of SEQ ID NOs: 2-21, 90 and SEQ ID NOs: 95-98, wherein the CDRs are annotated according to Kabat, Martin, MacCallum, IMGT, AbM, or Chothia, or wherein CDR1 is defined by any of SEQ ID NOs: 28-37, or 141-143, CDR2 is defined by any of SEQ ID NOs: 38-50, 144 or 145, and CDR3 is defined by any of SEQ ID NOs: 51-61, or 146; and
wherein the one or more second ISVDs comprise the complementarity determining regions (CDRs) present in any of SEQ ID NOs: 22-27 or SEQ ID NOs: 85-87, wherein the CDRs are annotated according to Kabat, Martin, MacCallum, IMGT, AbM, or Chothia, or wherein CDR1 is defined by SEQ ID NO: 70 or 138, CDR2 is defined by SEQ ID NO:71 or 139 and CDR3 is defined by SEQ ID NO:72 or 140; or wherein CDR1 is defined by any of SEQ ID NO: 62 or 63 or 131 or 132, CDR2 is defined by any of SEQ ID NO: 64-67 or 133-134 and CDR3 is defined by any of SEQ ID NO: 68 or 69 or 135-137.

2. The composition of claim 1, wherein the one or more second ISVDs allows the Angiotensin-Converting Enzyme 2 (ACE2)-receptor binding domain (RBD) of the Spike protein to bind to ACE2 when the one or more second ISVDs is bound to the RBD.

3. The composition of claim 1, wherein the one or more first ISVDs further bind to at least one or more of the amino acid residues of L368, S371, S375, T376, C379 and/or Y508 of the SARS-COV-2 spike protein as depicted in SEQ ID NO: 1.

4. The composition of claim 1, wherein the one or more second ISVDs further bind to at least one of the amino acid residues K462 (or alternatively R462 in some sarbecoviruses), F464 (or alternatively Y464 in some sarbecoviruses), E465 (or alternatively G465 in some sarbecoviruses) or R466.

5. The composition of claim 1, wherein the one or more second ISVDs further bind to at least the residue R357, of the SARS-COV-2 spike protein as depicted in SEQ ID NO:1.

6. The composition of claim 1, wherein the one or more second ISVDs bind to at least one of the amino acids N394 (or alternatively S394 in some sarbecoviruses), Y396, F464, S514, E516, and R355 of the SARS-COV-2 spike protein as defined in SEQ ID NO:1; and optionally further bind to amino acid R357 (or alternatively K357 in some sarbecoviruses) and/or K462 (or alternatively R462 in some sarbecoviruses) and/or E465 (or alternatively G465 in some sarbecoviruses) and/or R466 and/or L518.

7. The composition of claim 1, wherein the one or more second ISVDs induce S1 shedding.

8. (canceled)

9. (canceled)

10. The composition of claim 1, wherein the one or more first ISVDs comprise a sequence selected from the group of sequences consisting of SEQ ID NOs: 2-21, 90 and SEQ ID NOs: 95-98, or a functional variant with at least 90% identity thereof wherein the non-identical amino acids are located in one or more FRs, or a humanized variant thereof.

11. The composition of claim 1, wherein the one or more second ISVDs comprise a sequence selected from the group of sequences consisting of SEQ ID NO: 22-27 and SEQ ID NOs: 85-87 or a functional variant with at least 90% identity thereof wherein the non-identical amino acids are located in one or more FRs, or a humanized variant thereof.

12. The composition of claim 1, comprising at least one agent that competes with any of the binding agents selected from the group of SEQ ID NO: 2-21, 90 and SEQ ID NOs: 95-98 for its binding to the RBD, and/or comprising at least one agent that competes with any of the binding agents selected from the group consisting of SEQ ID NO: 22-27 and SEQ ID NOs: 85-87 for its binding to the RBD.

13. The composition of claim 1, comprising a single agent, wherein said agent comprises the one or more first ISVDs and the one or more second ISVDs.

14. The composition of claim 1, wherein said first and second ISVDs are fused directly or via a linker.

15. The composition of claim 14, wherein said linker is a short peptide linker or an Fc-tail or another moiety.

16. The composition of claim 1, wherein said agent comprises an IgG Fc to fuse said first and second ISVDs providing for a bispecific antibody, wherein said bispecific antibody may be bivalent or tetravalent such as a knob-into-hole VHH-fusion, a VHH-VHH-Fc fusion or a VHH-Fc-VHH fusion.

17. The composition of claim 12, wherein said agent comprises a sequence selected from the group of SEQ ID NOs: 76-84, SEQ ID NOs: 91-93, SEQ ID NO: 118, and SEQ ID NO: 119-121, or a functional variant with at least 90% identity thereof, or a humanized variant of any one thereof; or wherein said agent comprises a pair of sequences selected from the group of sequence pairs consisting of: SEQ ID NOs: 107 and 108, SEQ ID NOs: 109 and 110, SEQ ID NOs: 111 and 112, SEQ ID NOs: 113 and 114, SEQ ID NOs: 115 and 116, and SEQ ID NOs: 113 and 117, or a functional variant with at least 90% identity thereof, or a humanized variant of any one thereof.

18. A binding agent comprising one or more first ISVDs binding to the amino acid residues Y369, F377, and K378 of the SARS-COV-2 spike protein as depicted in SEQ ID NO:1, and one or more second ISVDs binding to at least one or more of the residues T393, N394, V395, or Y396 of the SARS-COV-2 spike protein as depicted in SEQ ID NO:1,
wherein the one or more first ISVDs comprise the complementarity determining regions (CDRs) as depicted in any of SEQ ID NOs: 2-21, 90 and SEQ ID NOs: 95-98, wherein the CDRs are annotated according to Kabat, Martin, MacCallum, IMGT, AbM, or Chothia, or wherein CDR1 is defined by any of SEQ ID NOs: 28-37, or 141-143, CDR2 is defined by any of SEQ ID NOS: 38-50, 144 or 145, and CDR3 is defined by any of SEQ ID NOs: 51-61, or 146; and
wherein the one or more second ISVDs comprise the complementarity determining regions (CDRs) present in any of SEQ ID NOs: 22-27 or SEQ ID NOs: 85-87, wherein the CDRs are annotated according to Kabat, Martin, MacCallum, IMGT, AbM, or Chothia, or wherein CDR1 is defined by SEQ ID NO: 70 or 138, CDR2 is defined by SEQ ID NO:71 or 139 and CDR3 is defined by SEQ ID NO:72 or 140; or wherein CDR1 is defined by any of SEQ ID NO: 62 or 63 or 131 or 132, CDR2 is defined by any of SEQ ID NO: 64-67 or 133-134 and CDR3 is defined by any of SEQ ID NO: 68 or 69 or 135-137.

19. An isolated nucleic acid encoding a binding agent according to claim 18.

20. A recombinant vector comprising the nucleic acid according to claim 19.

21. A pharmaceutical composition comprising the composition according to claim 1.

22. A method of treating a subject comprising administering to the subject the composition according to claim 1.

23. A method of passively immunizing a subject comprising administering to the subject the composition according to claim 1.

24. A method of treating a coronavirus infection in a subject comprising administering to the subject the composition according to claim 1.

25. A method of treating a SARS-COV-1 or SARS-COV-2 infection in a subject comprising administering to the subject the composition according to claim 1.
